(19) United States
(12) Patent Application Publication
(10) Pub. No.: US 2009/0030039 A1
(43) Pub. Date: Jan. 29, 2009

(54) Piperidine Derivatives as Cxcr3 Receptor Antagonists

(76) Inventors: Erwin Coesemans, Nijlen (BE); Jean-Pierre Andre Marc Bongartz, Turnhout (BE); Guy Rosali Eugeen Van Lommen, Berlaar (BE); Jean Pierre Frans Van Wauwe, Beerse (BE); Mieke Buntinx, Hasselt (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/278,719

(22) PCT Filed: Feb. 6, 2007
(86) PCT No.: PCT/EP2007/051106

§ 371 Date: Aug. 7, 2008

(30) Foreign Application Priority Data
Feb. 10, 2006 (EP) 06101560.8

(51) Int. Cl. A61K 31/4545; C07D 401/04

(52) U.S. Cl. 514/316; 546/188

(57) ABSTRACT

The present invention relates to a compound of formula (I)

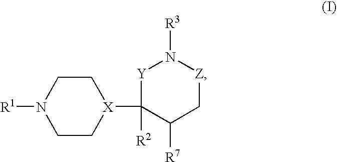

a N-oxide thereof, a pharmaceutically acceptable salt thereof, a stereochemically isomeric form thereof or a solvate thereof, wherein X represents N or CH; Y and Z each independently represent C(═O) or CH₂ provided that at least one of Y and Z represents C(═O); R¹ represents CH(R⁴)-aryl or CH(R⁴)-heteroaryl; R² represents aryl² or heteroaryl; R³ represents hydrogen; C₁₋₄alkylcarbonyl; C₁₋₆alkyl optionally substituted with C₁₋₆alkyloxy, C₁₋₆alkylthio, C₁₋₆alkyloxycarbonyl or aryl¹; provided that when Y and Z each represent C(═O), X represents CH, R³ represents hydrogen, R⁴ represents hydrogen, and R² represents unsubstituted pyridyl or phenyl optionally substituted with one halo or with one C₁₋₄alkyloxy or with one or two C₁₋₄alkyl, then aryl in the definition of R¹ is other than phenyl substituted with one halo or with one or two C₁₋₄alkyl; and
provided that when Y and Z each represent C(═O), X represents CH, R³ represents hydrogen, and R² represents unsubstituted pyridyl or phenyl optionally substituted with one halo or with one C₁₋₄alkyloxy or with one or two C₁₋₄alkyl, then heteroaryl in the definition of R¹ is other than unsubstituted thienyl or unsubstituted pyridyl.
The present invention also relates to the use of a compound of formula (I) for the manufacture of a medicament for preventing or treating a disease mediated through activation of the CXCR3 receptor; to processes for preparing the compounds of formula (I) and pharmaceutical compositions comprising them.

### FIELD OF THE INVENTION

The present invention concerns piperidine derivatives having CXCR3 receptor antagonistic properties. The invention further relates to methods for their preparation and pharmaceutical compositions comprising them. The invention also relates to the use of said compounds for the manufacture of a medicament for the prevention or the treatment of a disease mediated through activation of the CXCR3 receptor.

### BACKGROUND PRIOR ART

U.S. Pat. No. 3,125,578 describes 2,6-dioxo-piperidine derivatives having anticholinergic activity.

WO 95/11234 relates to N-derivatives of dexetimide suitable for PET studies and SPECT studies of muscarinic receptors.

The compounds of the invention differ from the prior art compounds in structure, in their pharmacological activity and/or pharmacological potency.

### DESCRIPTION OF THE INVENTION

One aspect of the present invention relates to a compound of formula

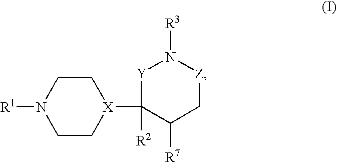

a N-oxide thereof, a pharmaceutically acceptable salt thereof, a stereochemically isomeric form thereof or a solvate thereof, wherein
X represents N or CH;
Y and Z each independently represent C(═O) or CH₂ provided that at least one of Y and Z represents C(═O);
R¹ represents CH(R⁴)-aryl or CH(R⁴)-heteroaryl;
R² represents aryl² or heteroaryl;
R³ represents hydrogen; C₁₋₄alkylcarbonyl; C₁₋₆alkyl optionally substituted with C₁₋₆alkyloxy, C₁₋₆alkylthio, C₁₋₆alkyloxycarbonyl or aryl¹;
R⁴ represents hydrogen or C₁₋₄alkyl;
R⁵ and R⁶ each independently represent hydrogen, or C₁₋₆alkyl optionally substituted with hydroxyl; or
R⁵ and R⁶ together with the nitrogen to which they are attached form a monocyclic heterocycle selected from piperidinyl, piperazinyl, morpholinyl, or thiomorpholinyl, each of said rings optionally substituted with C₁₋₄alkyl;
R⁷ represents hydrogen or C₁₋₄alkyl;
aryl represents unsubstituted naphthyl; or phenyl or naphthyl, each of said phenyl or naphthyl substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhalo-C₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—;
aryl¹ represents phenyl or phenyl substituted with 1, 2 or 3 substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, aminocarbonyl, mono- or di(C₁₋₄alkyl)aminocarbonyl, amino, or mono- or di(C₁₋₄alkyl)amino;
aryl² represents phenyl or naphthyl, each of said rings optionally substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹-C₁₋₄alkyloxy, aryl¹oxy, or aryl¹C(═O)—;
heteroaryl represents a monocyclic heterocycle selected from pyrrolinyl, imidazolinyl, pyrazolinyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, piperidinyl, piperazinyl, morpholinyl, thiomorpholinyl; or a bicyclic heterocycle selected from indolyl, indolizinyl, isoindolyl, indolinyl, benzofuranyl, benzothienyl, indazolyl, benzimidazolyl, benzthiazolyl, purinyl, quinolizinyl, quinolinyl, isoquinolinyl, cinnolinyl, phthalazinyl, quinazolinyl, quinoxalinyl, naphthyridinyl, pteridinyl, benzoxadiazolyl, benzoxazolyl, benzthiazolyl, each of said monocyclic or bicyclic heterocycle optionally being substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O), amino, mono- or di(C₁₋₄alkyl)amino or C₁₋₄alkylcarbonylamino;
   provided that when Y and Z each represent C(═O), X represents CH, R³ represents hydrogen, R⁴ represents hydrogen, and R² represents unsubstituted pyridyl or phenyl optionally substituted with one halo or with one C₁₋₄alkyloxy or with one or two C₁₋₄alkyl, then aryl in the definition of R¹ is other than phenyl substituted with one halo or with one or two C₁₋₄alkyl; and
   provided that when Y and Z each represent C(═O), X represents CH, R³ represents hydrogen, and R² represents unsubstituted pyridyl or phenyl optionally substituted with one halo or with one C₁₋₄alkyloxy or with one or two C₁₋₄alkyl, then heteroaryl in the definition of R¹ is other than unsubstituted thienyl or unsubstituted pyridyl.

The present invention also relates to a compound of formula

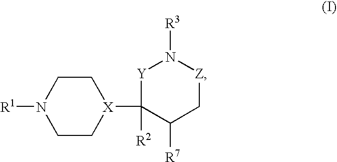

a N-oxide thereof, a pharmaceutically acceptable salt thereof, a stereochemically isomeric form thereof or a solvate thereof, wherein
X represents N or CH;
Y and Z each independently represent C(═O) or CH₂ provided that at least one of Y and Z represents C(═O);
R¹ represents CH(R⁴)-aryl or CH(R⁴)-heteroaryl;
R² represents aryl² or heteroaryl;
R³ represents hydrogen; C₁₋₄alkylcarbonyl; C₁₋₆alkyl optionally substituted with C₁₋₆alkyloxy, C₁₋₆alkylthio, C₁₋₆alkyloxycarbonyl or aryl¹;
R⁴ represents hydrogen or C₁₋₄alkyl;
R⁵ and R⁶ each independently represent hydrogen, or C₁₋₆alkyl optionally substituted with hydroxyl; or
R⁵ and R⁶ together with the nitrogen to which they are attached form a monocyclic heterocycle selected from piperidinyl, piperazinyl, morpholinyl, or thiomorpholinyl, each of said rings optionally substituted with C₁₋₄alkyl;
R⁷ represents hydrogen or C₁₋₄alkyl;
aryl represents unsubstituted naphthyl; or phenyl or naphthyl, each of said phenyl or naphthyl substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhalo-C₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—;
aryl¹ represents phenyl or phenyl substituted with 1, 2 or 3 substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, aminocarbonyl, mono- or di(C₁₋₄alkyl)aminocarbonyl, amino, or mono- or di(C₁₋₄alkyl)amino;
aryl² represents phenyl or naphthyl, each of said rings optionally substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹-C₁₋₄alkyloxy, aryl¹oxy, or aryl¹C(═O)—;
heteroaryl represents a monocyclic heterocycle selected from pyrrolinyl, imidazolinyl, pyrazolinyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, piperidinyl, piperazinyl, morpholinyl, thiomorpholinyl; or a bicyclic heterocycle selected from indolyl, indolizinyl, isoindolyl, indolinyl, benzofuranyl, benzothienyl, indazolyl, benzimidazolyl, benzthiazolyl, purinyl, quinolizinyl, quinolinyl, isoquinolinyl, cinnolinyl, phthalazinyl, quinazolinyl, quinoxalinyl, naphthyridinyl, pteridinyl, benzoxadiazolyl, benzoxazolyl, benzthiazolyl, each of said monocyclic or bicyclic heterocycle optionally being substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O), amino, mono- or di(C₁₋₄alkyl)amino or C₁₋₄alkylcarbonylamino;
   provided that when Y and Z each represent C(═O), X represents CH, R³ represents hydrogen, and R² represents unsubstituted pyridyl or phenyl optionally substituted with one halo or with one C₁₋₄alkyloxy or with one or two C₁₋₄alkyl, then heteroaryl in the definition of R¹ is other than unsubstituted thienyl or unsubstituted pyridyl and aryl in the definition of R¹ is other than phenyl substituted with one halo or with one or two C₁₋₄alkyl.

The present invention also relates to a compound selected from

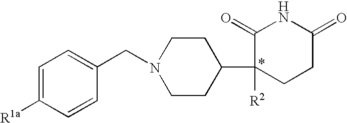

| R^{1a} | R² | stereochemistry/salt |
|---|---|---|
| —Br | phenyl | *R |
| —Br | phenyl | *R; •HCl |
| —Cl | 3-pyridyl | *RS |

a N-oxide thereof, a pharmaceutically acceptable salt thereof, a stereochemically isomeric form thereof or a solvate thereof.

The present invention also relates to the use of a compound of formula (I) for the manufacture of a medicament for preventing or treating a disease mediated through activation of the CXCR3 receptor, in particular for treating a disease mediated through activation of the CXCR3 receptor, in particular for preventing or treating, in particular for treating, an inflammatory disease, wherein the compound of formula (I) has the following formula

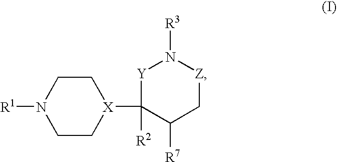

a N-oxide thereof, a pharmaceutically acceptable salt thereof, a stereochemically isomeric form thereof or a solvate thereof, wherein
X represents N or CH;
Y and Z each independently represent C(═O) or CH₂ provided that at least one of Y and Z represents C(═O);
R¹ represents CH(R⁴)-aryl or CH(R⁴)-heteroaryl;
R² represents aryl² or heteroaryl;
R³ represents hydrogen; C₁₋₄alkylcarbonyl; C₁₋₆alkyl optionally substituted with C₁₋₆alkyloxy, C₁₋₆alkylthio, C₁₋₆alkyloxycarbonyl or aryl¹;
R⁴ represents hydrogen or C₁₋₄alkyl;
R⁵ and R⁶ each independently represent hydrogen, or C₁₋₆alkyl optionally substituted with hydroxyl; or
R⁵ and R⁶ together with the nitrogen to which they are attached form a monocyclic heterocycle selected from piperidinyl, piperazinyl, morpholinyl, or thiomorpholinyl, each of said rings optionally substituted with C₁₋₄alkyl;
R⁷ represents hydrogen or C₁₋₄alkyl;
aryl represents unsubstituted naphthyl; or phenyl or naphthyl, each of said phenyl or naphthyl substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhalo-C₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—;
aryl¹ represents phenyl or phenyl substituted with 1, 2 or 3 substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, aminocarbonyl, mono- or di(C₁₋₄alkyl)aminocarbonyl, amino, or mono- or di(C₁₋₄alkyl)amino;
aryl² represents phenyl or naphthyl, each of said rings optionally substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹-C₁₋₄alkyloxy, aryl¹oxy, or aryl¹C(═O)—;
heteroaryl represents a monocyclic heterocycle selected from pyrrolinyl, imidazolinyl, pyrazolinyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, piperidinyl, piperazinyl, morpholinyl, thiomorpholinyl; or a bicyclic heterocycle selected from indolyl, indolizinyl, isoindolyl, indolinyl, benzofuranyl, benzothienyl, indazolyl, benzimidazolyl, benzthiazolyl, purinyl, quinolizinyl, quinolinyl, isoquinolinyl, cinnolinyl, phthalazinyl, quinazolinyl, quinoxalinyl, naphthyridinyl, pteridinyl, benzoxadiazolyl, benzoxazolyl, benzthiazolyl, each of said monocyclic or bicyclic heterocycle optionally being substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O), amino, mono- or di(C₁₋₄alkyl)amino or C₁₋₄alkylcarbonylamino.

As used hereinbefore or hereinafter C₁₋₄alkyl as a group or part of a group defines straight or branched chain saturated hydrocarbon radicals having from 1 to 4 carbon atoms such as methyl, ethyl, propyl, 1-methylethyl, butyl; C₁₋₆alkyl as a group or part of a group defines straight or branched chain saturated hydrocarbon radicals having from 1 to 6 carbon atoms such as the group defined for C₁₋₄alkyl and pentyl, hexyl, 2-methylbutyl and the like.

As used hereinbefore, the term (═O) forms a carbonyl moiety when attached to a carbon atom.

The term halo is generic to fluoro, chloro, bromo and iodo. As used hereinbefore or hereinafter, polyhaloC₁₋₆alkyl as a group or part of a group is defined as mono- or polyhalosubstituted C₁₋₆alkyl, for example methyl substituted with one or more fluoro atoms, for example, difluoromethyl or trifluoromethyl, 1,1-difluoro-ethyl and the like. In case more than one halogen atoms are attached to a C₁₋₆alkyl group within the definition of polyhaloC₁₋₆alkyl, they may be the same or different.

The term heteroaryl, e.g. in the definition of R¹ or R², is meant to include all the possible isomeric forms of the heterocycles, for instance, pyrrolyl comprises 1H-pyrrolyl and 2H-pyrrolyl.

The aryl, aryl¹, aryl² or heteroaryl listed in the definitions of the substituents of the compounds of formula (I) (see for instance R¹, R² and R³) as mentioned hereinbefore or hereinafter may be attached to the remainder of the molecule of formula (I) through any ring carbon or heteroatom as appropriate, if not otherwise specified. Thus, for example, when heteroaryl is pyridyl, it may be for instance 3-pyridyl or 4-pyridyl.

When any variable occurs more than one time in any constituent, each definition is independent.

For therapeutic use, salts of the compounds of formula (I) are those wherein the counterion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not are included within the ambit of the present invention.

The pharmaceutically acceptable salts as mentioned hereinbefore or hereinafter are meant to comprise the therapeutically active non-toxic acid addition salt forms which the compounds of formula (I) are able to form. The latter can conveniently be obtained by treating the base form with such appropriate acids as inorganic acids, for example, hydrohalic acids, e.g. hydrochloric, hydrobromic and the like; sulfuric acid; nitric acid; phosphoric acid and the like; or organic acids, for example, acetic, propanoic, hydroxy-acetic, 2-hydroxypropanoic, 2-oxopropanoic, oxalic, malonic, succinic, maleic, fumaric, malic, tartaric, 2-hydroxy-1,2,3-propanetricarboxylic, methanesulfonic, ethanesulfonic, benzenesulfonic, 4-methylbenzenesulfonic, cyclohexanesulfonic, 2-hydroxybenzoic, 4-amino-2-hydroxybenzoic and the like acids. Conversely the salt form can be converted by treatment with alkali into the free base form.

The compounds of formula (I) containing acidic protons may be converted into their therapeutically active non-toxic metal or amine addition salt forms by treatment with appropriate organic and inorganic bases. The pharmaceutically acceptable salts as mentioned hereinbefore or hereinafter are meant to also comprise the therapeutically active non-toxic metal or amine addition salt forms (base addition salt forms) which the compounds of formula (I) are able to form. Appropriate base addition salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline, the benzathine, N-methyl-D-glucamine, 2-amino-2-(hydroxymethyl)-1,3-propanediol, hydrabamine salts, and salts with amino acids such as, for example, arginine, lysine and the like.

Conversely the salt form can be converted by treatment with acid into the free acid form.

The term salt also comprises the quaternary ammonium salts (quaternary amines) which the compounds of formula (J) are able to form by reaction between a basic nitrogen of a compound of formula (I) and an appropriate quaternizing agent, such as, for example, an optionally substituted C₁₋₆alkylhalide, arylhalide, C₁₋₆alkylcarbonylhalide, arylcarbonylhalide, or arylC₁₋₆alkylhalide, e.g. methyliodide or benzyliodide. Other reactants with good leaving groups may also be used, such as for example C₁₋₆alkyl trifluoromethanesulfonates, C₁₋₆alkyl methanesulfonates, and C₁₋₆alkyl p-toluenesulfonates. A quaternary amine has a positively charged nitrogen. Pharmaceutically acceptable counterions include chloro, bromo, iodo, trifluoroacetate, acetate, triflate, sulfate, sulfonate. The counterion of choice can be introduced using ion exchange resins.

The term solvate comprises the hydrates and solvent addition forms which the compounds of formula (I) are able to form. Examples of such forms are e.g. hydrates, alcoholates and the like.

The N-oxide forms of the present compounds are meant to comprise the compounds of formula (I) wherein one or several tertiary nitrogen atoms are oxidized to the so-called N-oxide.

It will be appreciated that some of the compounds of formula (I) and their N-oxides, salts, stereochemically isomeric forms and solvates may contain one or more centers of chirality and exist as stereochemically isomeric forms.

The term “stereochemically isomeric forms” as used hereinbefore or hereinafter defines all the possible stereoisomeric forms which the compounds of formula (I), and their N-oxides, salts, solvates, or physiologically functional derivatives may possess. Unless otherwise mentioned or indicated, the chemical designation of compounds denotes the mixture of all possible stereochemically isomeric forms, said mixtures containing all diastereomers and enantiomers of the basic molecular structure as well as each of the individual isomeric forms of formula (I) and their N-oxides, salts or solvates, substantially free, i.e. associated with less than 10%, preferably less than 5%, in particular less than 2% and most preferably less than 1% of the other isomers. Thus, when a compound of formula (I) is for instance specified as R, this means that the compound is substantially free of the S isomer.

When a compound of formula (I) is specified as RS, this means that the compound is a mixture, in particular a racemic mixture, of the R and S isomers.

In particular, stereogenic centers may have the R- or S-configuration; substituents on bivalent cyclic (partially) saturated radicals may have either the cis- or trans-configuration. Compounds encompassing double bonds can have an E (entgegen) or Z (zusammen)-stereochemistry at said double bond. The terms cis, trans, R, S, E and Z are well known to a person skilled in the art.

Stereochemically isomeric forms of the compounds of formula (J) are obviously intended to be embraced within the scope of this invention.

Following CAS-nomenclature conventions, when two stereogenic centers of known absolute configuration are present in a molecule, an R or S descriptor is assigned (based on Cahn-Ingold-Prelog sequence rule) to the lowest-numbered chiral center, the reference center. The configuration of the second stereogenic center is indicated using relative descriptors [R*,R*] or [R*,S*], where the first R* is always specified as the reference center and [R*,R*] indicates centers with the same chirality and [R*,S*] indicates centers of unlike chirality. For example, if the lowest-numbered chiral center in the molecule has an S configuration and the second center is R, the stereo descriptor would be specified as S-[R*,S*]. If “ [symbol] ” and “®” are used: the position of the highest priority substituent on the asymmetric carbon atom in the ring system having the lowest ring number, is arbitrarily always in the “ [symbol] ” position of the mean plane determined by the ring system. The position of the highest priority substituent on the other asymmetric carbon atom in the ring system relative to the position of the highest priority substituent on the reference atom is denominated “ [symbol] ”, if it is on the same side of the mean plane determined by the ring system, or “®”, if it is on the other side of the mean plane determined by the ring system.

The compounds of (I) may be synthesized in the form of racemic mixtures of enantiomers which can be separated from one another following art-known resolution procedures. The racemic compounds of formula (I) may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound will be synthesized by stereospecific methods of preparation. These methods will advantageously employ enantiomerically pure starting materials.

Some of the compounds of formula (I) may also exist in their tautomeric form. Such forms although not explicitly indicated in the above formula (I) are intended to be included within the scope of the present invention.

The scope of the present invention also embraces all possible polymorphic forms which the compounds of formula (I) are able to form.

Whenever used hereinafter, the term “compounds of formula (I)” or any subgroup thereof, is meant to also include their N-oxide forms, their salts, their stereochemically isomeric forms and their solvates. Of special interest are those compounds of formula (I) which are stereochemically pure.

Whenever used hereinbefore or hereinafter that substituents can be selected each independently out of a list of numerous definitions, all possible combinations are intended which are chemically possible.

A first interesting embodiment of the present invention are those compounds of formula (I); a N-oxide thereof, a pharmaceutically acceptable salt thereof, a stereochemically isomeric form thereof or a solvate thereof, wherein
X represents N or CH;
Y and Z each independently represent C(═O) or CH₂ provided that at least one of Y and Z represents C(═O);
R¹ represents CH(R⁴)-aryl or CH(R⁴)-heteroaryl;
R² represents aryl² or heteroaryl;
R³ represents hydrogen; C₁₋₆alkyl optionally substituted with C₁₋₆alkyloxy, C₁₋₆alkylthio, C₁₋₆alkyloxycarbonyl or aryl¹;
R⁴ represents hydrogen or C₁₋₄alkyl;
R⁵ and R⁶ each independently represent hydrogen, or C₁₋₆alkyl optionally substituted with hydroxyl; or
R⁵ and R⁶ together with the nitrogen to which they are attached form a monocyclic heterocycle selected from piperidinyl, piperazinyl, morpholinyl, or thiomorpholinyl, each of said rings optionally substituted with C₁₋₄alkyl;
R⁷ represents hydrogen;
aryl represents unsubstituted naphthyl; or phenyl or naphthyl, each of said phenyl or naphthyl substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di-(C₁₋₄alkyl)amino, C₁₋₄alkylcarbonylamino, aryl¹, aryl¹C₁₋₄alkyloxy, aryl¹oxy, or aryl¹C(═O)—;
aryl¹ represents phenyl or phenyl substituted with 1, 2 or 3 substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, aminocarbonyl, mono- or di(C₁₋₄alkyl)aminocarbonyl, amino, or mono- or di(C₁₋₄alkyl)amino;
aryl² represents phenyl or naphthyl, each of said rings optionally substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—;
heteroaryl represents a monocyclic heterocycle selected from pyrrolinyl, imidazolinyl, pyrazolinyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, piperidinyl, piperazinyl, morpholinyl, thiomorpholinyl; or a bicyclic heterocycle selected from indolyl, indolizinyl, isoindolyl, indolinyl, benzofuranyl, benzothienyl, indazolyl, benzimidazolyl, benzthiazolyl, purinyl, quinolizinyl, quinolinyl, isoquinolinyl, cinnolinyl, phthalazinyl, quinazolinyl, quinoxalinyl, naphthyridinyl, pteridinyl, benzoxadiazolyl, benzoxazolyl, benzthiazolyl, each of said monocyclic or bicyclic heterocycle optionally being substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O), amino, mono- or di (C₁₋₄alkyl)amino or C₁₋₄alkylcarbonylamino;
   provided that when Y and Z each represent C(═O), X represents CH, R³ represents hydrogen, R⁴ represents hydrogen, and R² represents unsubstituted pyridyl or phenyl optionally substituted with one halo or with one C₁₋₄alkyloxy or with one or two C₁₋₄alkyl, then aryl in the definition of R¹ is other than phenyl substituted with one halo or with one or two C₁₋₄alkyl; and
   provided that when Y and Z each represent C(═O), X represents CH, R³ represents hydrogen, and R² represents unsubstituted pyridyl or phenyl optionally substituted with one halo or with one C₁₋₄alkyloxy or with one or two C₁₋₄alkyl, then heteroaryl in the definition of R¹ is other than unsubstituted thienyl or unsubstituted pyridyl.

A second interesting embodiment of the present invention are those compounds of formula (I) or any subgroup thereof as mentioned hereinbefore as interesting embodiment provided that when Y and Z each represent C(═O), X represents CH, R³ represents hydrogen, R⁴ represents hydrogen, R² represents unsubstituted phenyl, and aryl in the definition of R¹ represents optionally substituted phenyl, then the nitrogen atom carrying the R¹ substituent is not protonated or not quaternized. More preferably, the present invention relates to those compounds of formula (I) wherein the nitrogen atom carrying the R¹ substituent is not protonated or not quaternized.

A third interesting embodiment of the present invention are those compounds of formula (I) or any subgroup thereof as mentioned hereinbefore as interesting embodiment wherein Y represents CH₂

A fourth interesting embodiment of the present invention are those compounds of formula (I) or any subgroup thereof as mentioned hereinbefore as interesting embodiment wherein Z represents CH₂

A fifth interesting embodiment of the present invention are those compounds of formula (I) or any subgroup thereof as mentioned hereinbefore as interesting embodiment wherein both Y and Z represent C(═O).

A sixth interesting embodiment of the present invention are those compounds of formula (I) or any subgroup thereof as mentioned hereinbefore as interesting embodiment wherein X represents CH.

A seventh interesting embodiment of the present invention are those compounds of formula (I) or any subgroup thereof as mentioned hereinbefore as interesting embodiment wherein X represents N.

An eighth interesting embodiment of the present invention are those compounds of formula (I) or any subgroup thereof as mentioned hereinbefore as interesting embodiment wherein aryl represents unsubstituted naphthyl; or phenyl or naphthyl, each of said phenyl or naphthyl substituted with one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—.

A ninth interesting embodiment of the present invention are those compounds of formula (I) or any subgroup thereof as mentioned hereinbefore as interesting embodiment wherein R¹ represents CH(R⁴)-aryl, in particular wherein R¹ represents CH(R⁴)-aryl wherein aryl represents unsubstituted naphthyl; or phenyl or naphthyl, each of said phenyl or naphthyl substituted with one or two substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—.

A tenth interesting embodiment of the present invention are those compounds of formula (I) or any subgroup thereof as mentioned hereinbefore as interesting embodiment wherein R¹ represents CH(R⁴)-aryl wherein aryl represents phenyl substituted with at least one substituent, preferably one or two substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—; more in particular wherein R¹ represents CH(R⁴)-aryl wherein aryl represents phenyl substituted with one or two substituents, each substituent independently selected from halo, C₁₋₆alkyl, C₁₋₆alkyloxy, C₁₋₆alkyloxycarbonyl, C₁₋₆alkylthio, polyhaloC₁₋₆alkyl, polyhaloC₁₋₆alkyloxy, amino, nitro, aryl¹ or aryl¹-C₁₋₄alkyloxy; even more in particular wherein R¹ represents CH(R⁴)-aryl wherein aryl represents phenyl substituted with one or two halo. Preferably, R¹ represents 4-halobenzyl, in particular 4-halobenzyl wherein halo represents bromo or chloro.

An eleventh interesting embodiment of the present invention are those compounds of formula (I) or any subgroup thereof as mentioned hereinbefore as interesting embodiment wherein R¹ represents CH(R⁴)-aryl wherein aryl represents naphthyl optionally substituted with at least one substituent, preferably one or two substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—; in particular wherein R¹ represents CH(R⁴)-aryl wherein aryl represents unsubstituted naphthyl.

A twelfth interesting embodiment of the present invention are those compounds of formula (I) or any subgroup thereof as mentioned hereinbefore as interesting embodiment wherein R¹ represents CH(R⁴)-aryl with aryl representing substituted phenyl wherein phenyl when carrying 1 substituent, is substituted preferably in position 3 or 4, or when carrying 2 substituents, is substituted preferably in position 3 and 4. Preferred substituents are halo, in particular bromo or chloro.

A thirteenth interesting embodiment of the present invention are those compounds of formula (I) or any subgroup thereof as mentioned hereinbefore as interesting embodiment wherein R¹ represents CH(R⁴)-heteroaryl; in particular wherein R¹ represents CH(R⁴)-heteroaryl wherein heteroaryl represents a monocyclic heterocycle selected from pyrrolinyl, imidazolinyl, pyrazolinyl, furanyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, piperidinyl, piperazinyl, morpholinyl, thiomorpholinyl; or a bicyclic heterocycle selected from indolyl, indolizinyl, isoindolyl, indolinyl, benzofuranyl, benzothienyl, indazolyl, benzimidazolyl, benzthiazolyl, purinyl, quinolizinyl, quinolinyl, isoquinolinyl, cinnolinyl, phthalazinyl, quinazolinyl, quinoxalinyl, naphthyridinyl, pteridinyl, benzoxadiazolyl, benzoxazolyl, benzthiazolyl, each of said monocyclic or bicyclic heterocycle optionally being substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino or C₁₋₄alkylcarbonylamino; more in particular wherein R¹ represents CH(R⁴)-heteroaryl wherein heteroaryl represents a monocyclic heterocycle selected from pyridyl, pyridazinyl, pyrimidinyl, pyrazinyl, piperidinyl, piperazinyl, morpholinyl, thiomorpholinyl; or a bicyclic heterocycle selected from indolyl, indolizinyl, isoindolyl, indolinyl, benzofuranyl, benzothienyl, indazolyl, benzimidazolyl, benzthiazolyl, purinyl, quinolizinyl, quinolinyl, isoquinolinyl, cinnolinyl, phthalazinyl, quinazolinyl, quinoxalinyl, naphthyridinyl, pteridinyl, benzoxadiazolyl, benzoxazolyl, benzthiazolyl, each of said monocyclic or bicyclic heterocycle optionally being substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino or C₁₋₄alkylcarbonylamino; even more in particular wherein R¹ represents CH(R⁴)-heteroaryl wherein heteroaryl represents a bicyclic heterocycle selected from indolyl, indolizinyl, isoindolyl, indolinyl, benzofuranyl, benzothienyl, indazolyl, benzimidazolyl, benzthiazolyl, purinyl, quinolizinyl, quinolinyl, isoquinolinyl, cinnolinyl, phthalazinyl, quinazolinyl, quinoxalinyl, naphthyridinyl, pteridinyl, benzoxadiazolyl, benzoxazolyl, benzthiazolyl, each of said bicyclic heterocycle optionally being substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhalo-C₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino or C₁₋₄alkylcarbonylamino.

A fourteenth interesting embodiment of the present invention are those compounds of formula (I) or any subgroup thereof as mentioned hereinbefore as interesting embodiment wherein R¹ represents CH(R⁴)-heteroaryl and the ring system representing heteroaryl is optionally substituted with one or two substituents, preferably one substituent.

A fifteenth interesting embodiment of the present invention are those compounds of formula (I) or any subgroup thereof as mentioned hereinbefore as interesting embodiment wherein R² represents aryl² or heteroaryl wherein heteroaryl is as defined hereinabove and aryl² represents unsubstituted naphthyl; or phenyl or naphthyl, each of said phenyl or naphthyl substituted with at least one substituent, preferably one or two, substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhalo-C₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)— or wherein R² represents aryl² wherein aryl² represents phenyl optionally substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—; in particular wherein R² represents aryl² wherein aryl² represents unsubstituted naphthyl; or phenyl or naphthyl, each of said phenyl or naphthyl substituted with at least one substituent, preferably one or two, substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhalo-C₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹-C₁₋₄alkyloxy, aryl¹oxy, or aryl¹C(═O)—; more in particular wherein R² represents aryl² wherein aryl² represents phenyl substituted with at least one substituent, preferably one or two substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—; even more in particular wherein R² represents aryl² wherein aryl² represents phenyl substituted with at least one substituent, preferably one or two substituents, each substituent independently selected from halo, hydroxy, C₁₋₆alkyl, C₁₋₆alkyloxy, polyhaloC₁₋₆alkyl, nitro, carboxyl, HO—SO₂—, R⁶R⁵N—C(═O)—, amino, or C₁₋₄alkylcarbonylamino; most in particular wherein R² represents aryl² wherein aryl² represents phenyl substituted with one or two substituents, each substituent independently selected from halo, C₁₋₆alkyloxy, carboxyl, HO—SO₂—, R⁶R⁵N—C(═O)—, or amino.

A sixteenth interesting embodiment of the present invention are those compounds of formula (I) or any subgroup thereof as mentioned hereinbefore as interesting embodiment wherein R² represents phenyl substituted with one substituent, said substituent preferably being placed in position 2, 3 or 4, or wherein R² represents phenyl substituted with two substituents, said substituents preferably being placed in position 2 and 4. Preferably, said substituents are halo, in particular fluoro.

A seventeenth interesting embodiment of the present invention are those compounds of formula (I) or any subgroup thereof as mentioned hereinbefore as interesting embodiment wherein R² represents heteroaryl; in particular wherein R² represents heteroaryl wherein heteroaryl represents a monocyclic heterocycle selected from pyrrolinyl, imidazolinyl, pyrazolinyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, piperidinyl, piperazinyl, morpholinyl, thiomorpholinyl; each of said monocyclic heterocycle optionally being substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhalo-C₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino or C₁₋₄alkylcarbonylamino; in particular wherein R² represents heteroaryl wherein heteroaryl represents a monocyclic heterocycle selected from pyrrolinyl, imidazolinyl, pyrazolinyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridazinyl, pyrimidinyl, pyrazinyl, piperidinyl, piperazinyl, morpholinyl, thiomorpholinyl; each of said monocyclic heterocycle optionally being substituted with at least one substituent, in particular one, two or three substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino or C₁₋₄alkylcarbonylamino.

An eighteenth interesting embodiment of the present invention are those compounds of formula (I) or any subgroup thereof as mentioned hereinbefore as interesting embodiment wherein R³ represents C₁₋₄alkylcarbonyl; C₁₋₆alkyl optionally substituted with C₁₋₆alkyloxy, C₁₋₆alkylthio, C₁₋₆alkyloxycarbonyl or aryl¹.

A nineteenth interesting embodiment of the present invention are those compounds of formula (I) or any subgroup thereof as mentioned hereinbefore as interesting embodiment wherein R³ represents hydrogen.

A twentieth interesting embodiment of the present invention are those compounds of formula (I) or any subgroup thereof as mentioned hereinbefore as interesting embodiment wherein R⁴ represents hydrogen.

A twenty first interesting embodiment of the present invention are those compounds of formula (I) or any subgroup thereof as mentioned hereinbefore as interesting embodiment wherein R⁴ represents C₁₋₄alkyl.

A twenty second interesting embodiment of the present invention are those compounds of formula (I) or any subgroup thereof as mentioned hereinbefore wherein aryl¹ represents phenyl or phenyl substituted with halo.

A twenty third interesting embodiment of the present invention are those compounds of formula (I) or any subgroup thereof as mentioned hereinbefore as interesting embodiment wherein R⁷ represents hydrogen.

A twenty fourth interesting embodiment of the present invention are those compounds of formula (I) or any subgroup thereof as mentioned hereinbefore as interesting embodiment wherein R⁷ represents C₁₋₄alkyl.

A twenty fifth interesting embodiment of the present invention are those compounds of formula (I) or any subgroup thereof as mentioned hereinbefore as interesting embodiment wherein the carbon atom carrying the R² and the

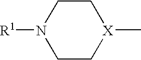

moiety has the R configuration, i.e. compounds having the following formula

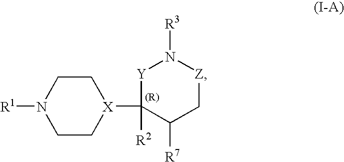

a N-oxide thereof, a pharmaceutically acceptable salt thereof, a stereochemically isomeric form thereof or a solvate thereof.

This group of compounds is a preferred group because the compounds show less anticholinergic side effects.

A twenty sixth interesting embodiment of the present invention are those compounds of formula (I) or any subgroup thereof as mentioned hereinbefore as interesting embodiment wherein one or more, preferably all, of the following restrictions apply:
a) R³ represents hydrogen; C₁₋₄alkylcarbonyl; C₁₋₆alkyl optionally substituted with C₁₋₆alkyloxy, C₁₋₆alkylthio, C₁₋₆alkyloxycarbonyl or aryl¹;
b) aryl represents unsubstituted naphthyl; or phenyl substituted with at least one substituent, in particular 1, 2 or 3 substituents, each substituent independently selected from halo, C₁₋₆alkyl, C₁₋₆alkyloxy, C₁₋₆alkyloxycarbonyl, C₁₋₆alkylthio, polyhaloC₁₋₆alkyl, polyhaloC₁₋₆alkyloxy, nitro, amino, aryl¹, or aryl¹C₁₋₄alkyloxy;
c) aryl¹ represents phenyl or phenyl substituted with halo;
d) aryl² represents phenyl, optionally substituted with at least one substituent, in particular 1 or 2 substituents, each substituent independently selected from halo, hydroxyl, C₁₋₆alkyl, C₁₋₆alkyloxy, polyhaloC₁₋₆alkyl, nitro, carboxyl, HO—SO₂—, R⁶R⁵N—C(═O)—, amino, or C₁₋₄alkylcarbonylamino;
e) heteroaryl represents thienyl, pyridyl, benzofuranyl, benzoxadiazolyl, each of said ring systems optionally being substituted with halo;
f) R⁵ and R⁶ each independently represent hydrogen, or C₁₋₆alkyl optionally substituted with hydroxyl; or R⁵ and R⁶ together with the nitrogen to which they are attached form a monocyclic heterocycle selected from piperazinyl or morpholinyl, each of said rings optionally substituted with C₁₋₄alkyl;
g) Y and Z are both C(═O); Y is CH₂ and Z is C(═O); Y is C(═O) and Z is CH₂;
h) X is CH or N;
i) R⁷ represents hydrogen or methyl;
j) the compound is a compound of formula (I-A).

A twenty seventh interesting embodiment of the present invention are those compounds of formula (I) or any subgroup thereof as mentioned hereinbefore as interesting embodiment wherein one or more, preferably all, of the following restrictions apply:
a) R³ represents hydrogen; or C₁₋₆alkyl optionally substituted with C₁₋₆alkylthio or C₁₋₆alkyloxycarbonyl;
b) aryl represents phenyl substituted with 1, 2 or 3 substituents, each substituent independently selected from halo, C₁₋₆alkyl, C₁₋₆alkyloxycarbonyl, polyhaloC₁₋₆alkyl, or polyhaloC₁₋₆alkyloxy;
d) aryl² represents phenyl optionally substituted with at least one substituent, in particular 1 or 2 substituents, each substituent independently selected from halo, hydroxyl, C₁₋₆alkyloxy, carboxyl, HO—SO₂—, R⁶R⁵N—C(═O)—, amino, or C₁₋₄alkylcarbonylamino; with R⁵ and R⁶ each representing hydrogen or R⁵ and R⁶ together with the nitrogen to which they are attached form morpholinyl;
e) heteroaryl represents pyridyl, benzoxadiazolyl or benzofuranyl substituted with halo;
f) Y and Z are both C(═O); Y is CH₂ and Z is C(═O); Y is C(═O) and Z is CH₂;
g) X is CH or N;
h) R⁷ represents hydrogen or methyl;
i) the compound is a compound of formula (I-A).

Preferred compounds of formula (I) are selected from the following compounds

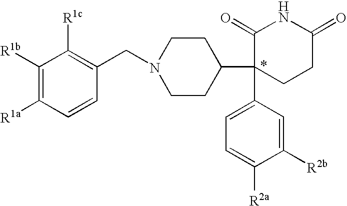

| | | | | | stereo- |
|---|---|---|---|---|---|
| | | | | | chemistry/ |
| R^{1a} | R^{1b} | R^{1c} | R^{2a} | R^{2b} | salt |
| —Br | H | H | H | —COOH | *RS |
| —Cl | —F | H | H | H | *S; (+) |
| —Cl | —F | H | H | H | *R; (−) |
| —Cl | —F | H | H | —COOH | *RS |
| —Cl | —F | H | —F | H | *RS |
| —Br | H | H | H | H | (±) |
| —Br | H | H | H | H | *R |
| —Br | H | H | H | H | *R; •HCl |
| —Br | H | H | H | H | *S |
| —Br | H | H | H | 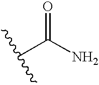 | *RS |
| —Br | H | H | H | —NH₂ | *RS |
| —Br | —F | H | H | H | *R |
| —Br | —F | H | H | —COOH | *RS |
| —Cl | —F | H | H | H | *RS |

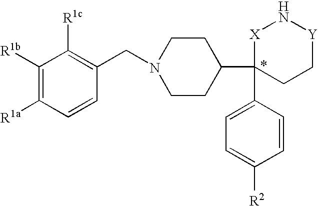

| | | | | | | Stereo- |
|---|---|---|---|---|---|---|
| R^{1a} | R^{1b} | R^{1c} | R² | X | Y | chemistry |
| —Br | H | —F | —F | —CH₂— | 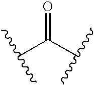 | *RS |

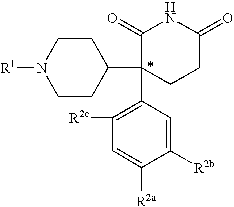

| | | | | stereo- |
|---|---|---|---|---|
| R¹ | R^{2a} | R^{2b} | R^{2c} | chemistry |
| 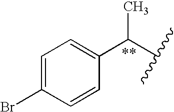 | F | H | F | *RS;**RS |
| 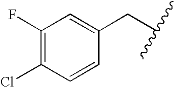 | F | H | F | *RS |
| 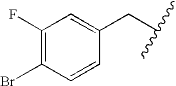 | F | H | F | *RS |
| 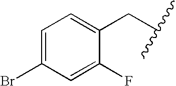 | F | H | F | *RS |
| 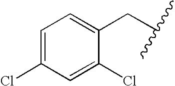 | F | H | F | *RS |
| 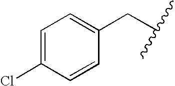 | F | H | F | *RS |
| 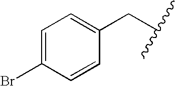 | F | H | F | *RS |
| 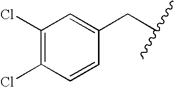 | F | H | F | *RS |
| 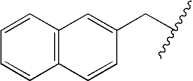 | H | —Cl | H | *RS |
| 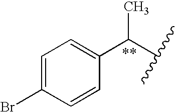 | H | H | H | *R;** R or S |
| 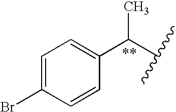 | H | H | H | *R;** S or R |
| 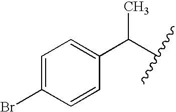 | —F | H | H | *RS |
| 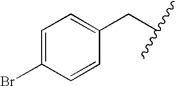 | H | —SO₃H | —OCH₃ | *RS |

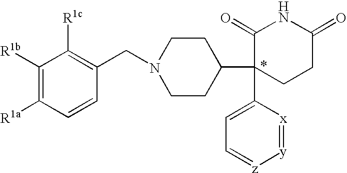

| | | | | | | Stereo- |
|---|---|---|---|---|---|---|
| x | y | z | R^{1a} | R^{1b} | R^{1c} | chemistry |
| C | N | C | —Cl | H | H | *RS |

^{a}N-oxide thereof, a pharmaceutically acceptable salt thereof, a stereochemically isomeric form thereof or a solvate thereof.

In particular, preferred compounds are selected from

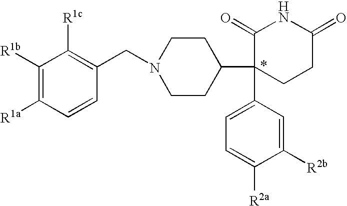

| | | | | | stereo- |
|---|---|---|---|---|---|
| R^{1a} | R^{1b} | R^{1c} | R^{2a} | R^{2b} | chemistry |
| —Br | H | H | H | —COOH | *RS |
| —Cl | —F | H | H | H | *S; (+) |
| —Cl | —F | H | H | H | *R; (−) |
| —Cl | —F | H | H | —COOH | *RS |
| —Cl | —F | H | —F | H | *RS |
| —Br | H | H | H | 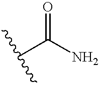 | *RS |
| —Br | H | H | H | —NH₂ | *RS |
| —Br | —F | H | H | H | *R |
| —Br | —F | H | H | —COOH | *RS |
| —Cl | —F | H | H | H | *RS |

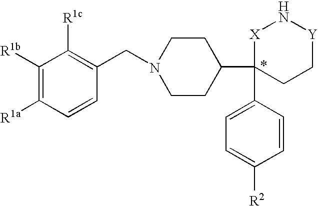

| | | | | | | Stereo- |
|---|---|---|---|---|---|---|
| R^{1a} | R^{1b} | R^{1c} | R² | X | Y | chemistry |
| —Br | H | —F | —F | —CH₂— | 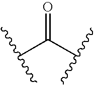 | *RS |

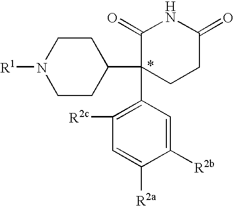

| | | | | stereo- |
|---|---|---|---|---|
| R¹ | R^{2a} | R^{2b} | R^{2c} | chemistry |
| 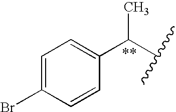 | F | H | F | *RS;**RS |
| 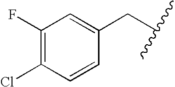 | F | H | F | *RS |
| 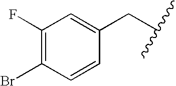 | F | H | F | *RS |
| 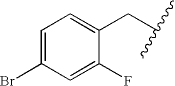 | F | H | F | *RS |
| 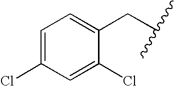 | F | H | F | *RS |
| 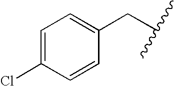 | F | H | F | *RS |
| 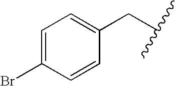 | F | H | F | *RS |
| 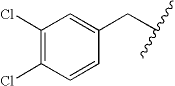 | F | H | F | *RS |
| 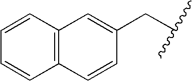 | H | —Cl | H | *RS |
| 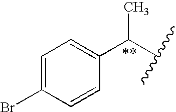 | H | H | H | *R;** R or S |
| 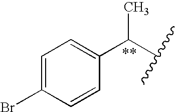 | H | H | H | *R;** S or R |
| 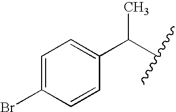 | —F | H | H | *RS;**RS |
| 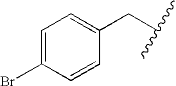 | H | —SO₃H | —OCH₃ | *RS |

^{a}N-oxide thereof, a pharmaceutically acceptable salt thereof, a stereochemically isomeric form thereof or a solvate thereof.

More in particular, preferred compounds are selected from

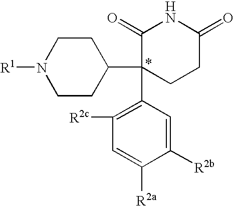

| R¹ | R^{2a} | R^{2b} | R^{2c} | stereochemistry |
|---|---|---|---|---|
| 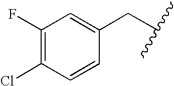 | F | H | F | *RS |
| 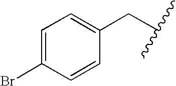 | F | H | F | *RS |
| 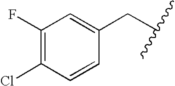 | F | H | F | *R; HCl |
| 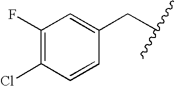 | F | H | F | *S; HCl |
| 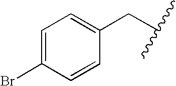 | F | H | F | *R; HCl |
| 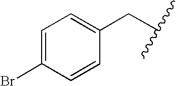 | F | H | F | *S; HCl |

a N-oxide thereof, a pharmaceutically acceptable salt thereof, a stereochemically isomeric form thereof or a solvate thereof

In general, compounds of formula (I) can be prepared by reacting an intermediate of formula (II) with an intermediate of formula (III) wherein W₁ represents a suitable leaving group, such as for example halo, e.g. chloro, bromo and the like, in the presence of a suitable solvent, such as for example N,N-dimethylformamide, dichloromethane, tetrahydrofuran, an alcohol, e.g. methanol and the like, and a suitable base, such as for example N,N-diethylethanamine, N,N-diisopropylethanamine, K₂CO₃ or NaHCO₃

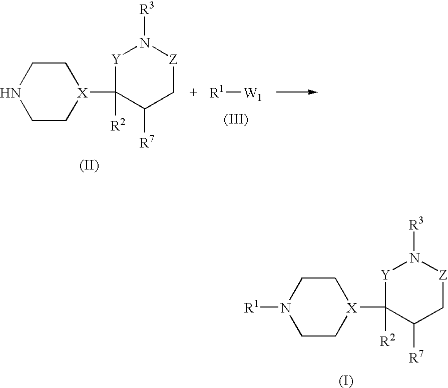

The above reaction can also be used to introduce at the same time a R³ substituent different from hydrogen and which is encompassed by the definition of the R¹ substituent.

The intermediates of formula (II) contain a chiral center at the carbon atom carrying the R² substituent. In case the intermediate of formula (II) is a stereospecific intermediate, the above reaction results in the formation of a stereospecific compound of formula (I).

Compounds of formula (I) wherein R³ represents hydrogen, wherein both Y and Z represent C(═O), and wherein X represents CH, said compounds being represented by formula (I-a-1), can also be prepared by reacting an intermediate of formula (IV) with a suitable acid, such as for example acetic acid and sulfuric acid.

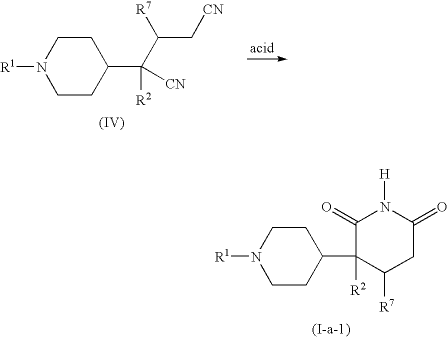

Compounds of formula (I-a-1) wherein R¹ represents CH(R⁴)-phenyl wherein phenyl is substituted with nitro and wherein R² represents phenyl substituted with nitro, said compounds being represented by formula (I-a-1-1), can be prepared by reacting an intermediate of formula (XXXV) with HNO₃

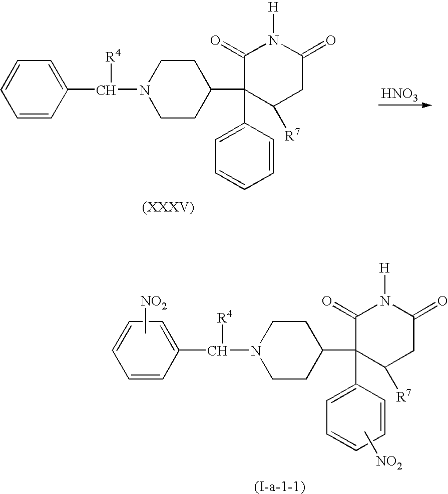

Compounds of formula (I) wherein the R⁴ substituent in the definition of R¹ represents hydrogen, said R¹ being represented by R^{1a}—CH₂ and said compounds being represented by formula (I-a-2), can be prepared by reacting an intermediate of formula (II) with an intermediate of formula (V) wherein R^{1a} represents aryl or heteroaryl, in the presence of a suitable reducing agent, such as for example NaBH(OAc)₃, a suitable acid, such as for example acetic acid, and a suitable solvent, such as for example dichloromethane.

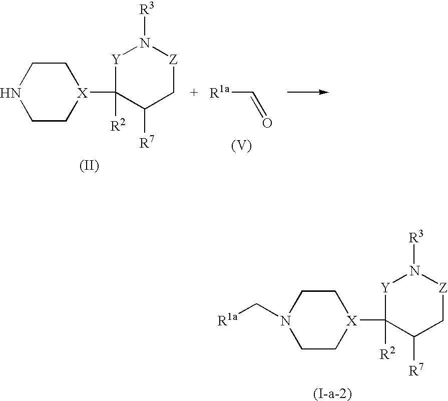

As already stated above, the intermediates of formula (II) contain a chiral center at the carbon atom carrying the R² substituent. In case the intermediate of formula (II) is a stereospecific intermediate, the above reaction results in the formation of a stereospecific compound of formula (I-a-2).

Compounds of formula (I) wherein the ring moiety of the R² substituent is substituted with R⁵R⁶N—C(═O)—, said R² substituent being represented by —R^{2a}—C(═O)—NR⁵R⁶ and said compounds being represented by formula (I-b-1), or compounds of formula (I) wherein the ring moiety of the R¹ substituent is substituted with R⁵R⁶N—C(═O)—, said R¹ substituent being represented by —R^{1a}—C(═O)—NR⁵R⁶ and said compounds being represented by formula (I-b-2), can be prepared by reacting an intermediate of formula (VI-a) or (VI-b) wherein W₂ represents a suitable leaving group, such as for example halo, e.g. chloro, or 1H-imidazolyl or azide and the like, with a suitable base of formula R⁵R⁶NH in the presence of a suitable solvent, such as for example dioxane or an alcohol, e.g. ethanol, methanol and the like.

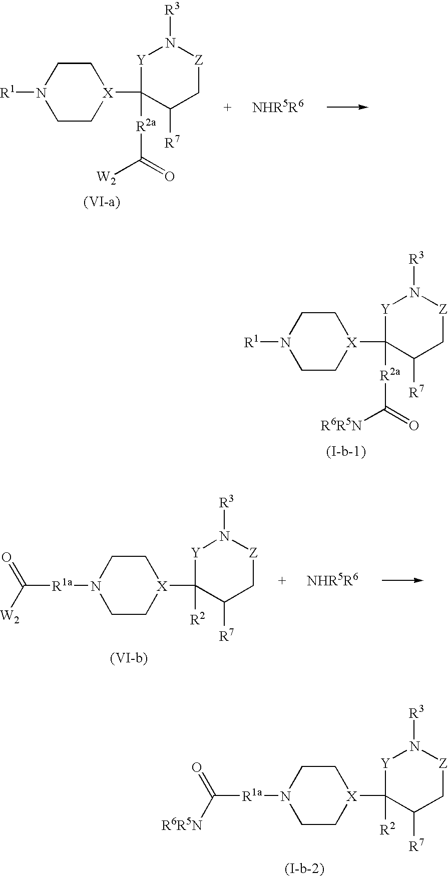

Compounds of formula (I) wherein the ring moiety of the R² substituent is substituted with NH₂, said R² substituent being represented by —R^{2a}—NH₂ and said compounds being represented by formula (I-c), can be prepared by deprotecting an intermediate of formula (VII) wherein P represents a suitable protecting group, such as for example tert BuOC(═O)—, with a suitable acid, such as for example trifluoroacetic acid, in the presence of a suitable solvent, such as for example dichloromethane.

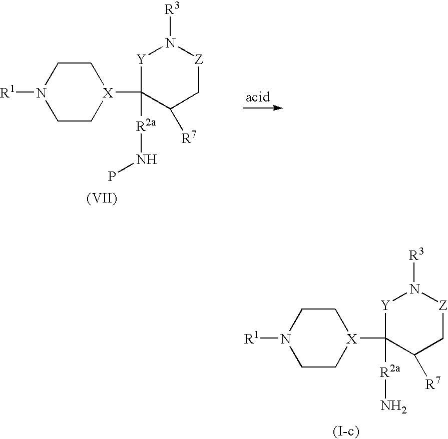

Compounds of formula (I) wherein the ring moiety of the R¹ or R² substituent is substituted with C₁₋₆alkyloxycarbonyl, said R¹ substituent being represented by —R^{1a}—C(═O)—O—C₁₋₆alkyl or said R² substituent being represented by —R^{2a}—C(═O)—O—C₁₋₆alkyl and said compounds being represented by formula (I-d) or (I-e), can be prepared by reacting an intermediate of formula (VIII) wherein W₃ represents a suitable leaving group, such as for example halo, e.g. chloro and the like, or an intermediate of formula (VI) with a suitable alcohol of formula C₁₋₆alkyl-OH.

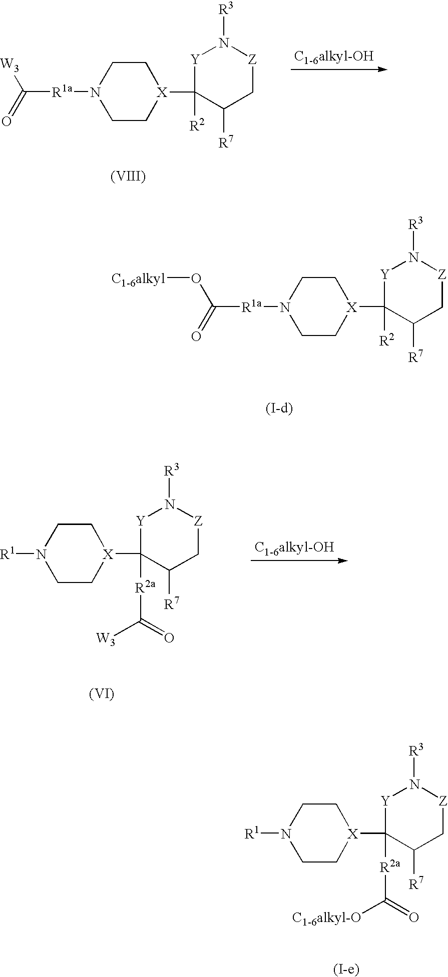

Compounds of formula (I) wherein R³ represents hydrogen, said compounds being represented by formula (I-a), can be converted into a compound of formula (I) wherein R³ represents C₁₋₆alkyl optionally substituted with C₁₋₆alkyloxy, C₁₋₆alkylthio, C₁₋₆alkyloxycarbonyl or aryl¹, said R³ substituent being represented by R^{3a} and said compounds being represented by formula (I-f), by reaction with W₄—R^{3a} wherein W₄ represents a suitable leaving group, such as for example halo, e.g. chloro, bromo, iodo and the like, in the presence of a suitable base, such as for example K₂CO₃, and a suitable solvent, such as for example N,N-dimethylformamide.

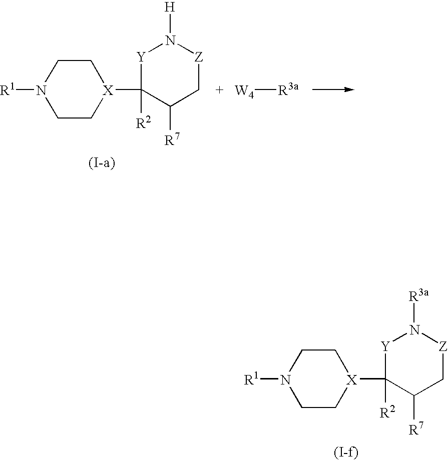

The compounds of formula (I) may further be prepared by converting compounds of formula (I) into each other according to art-known group transformation reactions.

The compounds of formula (I) may be converted to the corresponding N-oxide forms following art-known procedures for converting a trivalent nitrogen into its N-oxide form. Said N-oxidation reaction may generally be carried out by reacting the starting material of formula (I) with an appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chlorobenzenecarboperoxoic acid, peroxoalkanoic acids, e.g. peroxoacetic acid, alkylhydroperoxides, e.g. tert.butyl hydro-peroxide. Suitable solvents are, for example, water, lower alcohols, e.g. ethanol and the like, hydrocarbons, e.g. toluene, ketones, e.g. 2-butanone, halogenated hydrocarbons, e.g. dichloromethane, and mixtures of such solvents.

Compounds of formula (I) wherein the ring moiety of the R¹ or the R² substituent is substituted with methoxy, can be converted into a compound of formula (I) wherein the ring moiety of the R¹ or R² substituent is substituted with hydroxyl, by reaction with a suitable dealkylating agent, such as for example BBr₃, in the presence of a suitable solvent, such as for example dichloromethane.

Compounds of formula (I) wherein the ring moiety of the R¹ or the R² substituent is substituted with carboxyl, can be converted into a compound of formula (I) wherein the ring moiety of the R¹ or the R² substituent is substituted with —C(═O)—NR⁵R⁶, by reaction with a suitable amine HNR⁵R⁶ in the presence of a suitable solvent, such as for example dichloromethane, a suitable coupling agent, such as diimidazolylcarbonyl or carbodiimides, e.g. diisopropylcarbodiimide, 1-(3-dimethylaminopropyl)-3-ethylcarbodiimide, N,N′-dicyclohexylcarbodiimide, and optionally a suitable base, such as for example N,N-diisopropylethanamine. This conversion can also be achieved by first converting the carboxylic acid into an acylhalide by reaction with SOCl₂optionally in the presence of a suitable solvent, such as for example toluene, dichloromethane followed by the above-described reaction.

Compounds of formula (I) wherein the ring moiety of the R¹ or the R² substituent is substituted with nitro, can be converted into a compound of formula (I) wherein the ring moiety of the R¹ or the R² substituent is substituted with amino, by reaction with a suitable reducing agent, such as for example H₂, in the presence of a suitable catalyst, such as for example platina on charcoal, a suitable catalyst poison, such as for example a thiophene solution, V₂O₅ and a suitable solvent, such as for example tetrahydrofuran.

Compounds of formula (I) wherein the ring moiety of the R¹ or the R² substituent is substituted with nitro, can be prepared from the unsubstituted compound by reaction with HNO₃.

Compounds of formula (I) wherein the ring moiety of the R¹ or the R² substituent is substituted with amino, can be converted into a compound of formula (I) wherein the ring moiety of the R¹ or the R² substituent is substituted with C₁₋₆alkylcarbonylamino, by reaction with a suitable anhydride O(C(═O)—C₁₋₆alkyl)₂ or a suitable acyl chloride C₁₋₆alkyl-C(═O)—Cl in the presence of a suitable solvent, such as for example dichloromethane, and a suitable base, such as for example N,N-diisopropylethanamine.

Compounds of formula (I) wherein R³ represents hydrogen, can be converted into a compound of formula (I) wherein R³ represents C₁₋₄alkylcarbonyl by reaction with for instance acetic acid anhydride or acetyl chloride.

Some of the compounds of formula (I) and some of the intermediates in the present invention may contain an asymmetric carbon atom. Pure stereochemically isomeric forms of said compounds and said intermediates can be obtained by the application of art-known procedures. For example, diastereoisomers can be separated by physical methods such as selective crystallization or chromatographic techniques, e.g. counter current distribution, chiral liquid chromatography and the like methods. Enantiomers can be obtained from racemic mixtures by first converting said racemic mixtures with suitable resolving agents such as, for example, chiral acids, to mixtures of diastereomeric salts or compounds; then physically separating said mixtures of diastereomeric salts or compounds by, for example, selective crystallization or chromatographic techniques, e.g. liquid chromatography and the like methods; and finally converting said separated diastereomeric salts or compounds into the corresponding enantiomers. Pure stereochemically isomeric forms may also be obtained from the pure stereochemically isomeric forms of the appropriate intermediates and starting materials, provided that the intervening reactions occur stereospecifically.

An alternative manner of separating the enantiomeric forms of the compounds of formula (I) and intermediates involves liquid chromatography or SCF (Super Critical Fluid) chromatography, in particular using a chiral stationary phase.

Some of the intermediates and starting materials are known compounds and may be commercially available or may be prepared according to art-known procedures.

Intermediates of formula (II) wherein Y and Z represent C(═O) and R³ represents hydrogen, said intermediates being represented by formula (II-a), can be prepared by debenzylating an intermediate of formula (IX) in the presence of H₂, a suitable catalyst, such as for example palladium on charcoal, and in the presence of a suitable solvent, such as for example an alcohol, e.g. methanol and the like, or acetic acid in water. The debenzylation of an intermediate of formula (IX) can also be performed in the presence of 1-chloroethylcarbonochloridic acid ester, a suitable base, such as for example N,N-diisopropylethanamine, and a suitable solvent, such as for example dichloroethane followed by addition of an alcohol, e.g. methanol and the like, preferably at elevated temperatures.

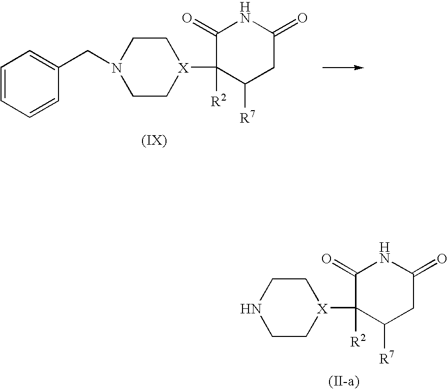

Compounds of formula (I-a-1) can also be converted into an intermediate of formula (II-a) by reaction with H₂, a suitable catalyst, such as for example palladium on charcoal, and a suitable solvent, such as for example tetrahydrofuran or an alcohol, e.g. methanol and the like.

Intermediates of formula (II-a) wherein X represents N, said intermediates being represented by formula (II-a-1), can also be prepared by reacting an intermediate of formula (X) with a suitable acid, such as for example hydrochloric acid and the like, in the presence of a suitable solvent, such as for example an alcohol, e.g. 2-propanol and and like.

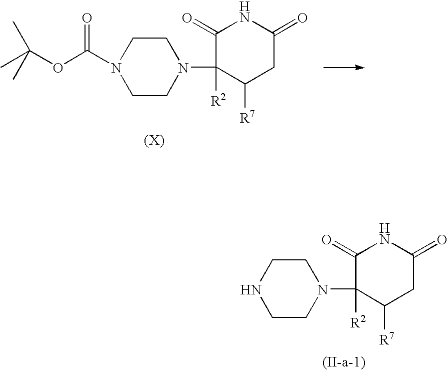

Intermediates of formula (II) wherein Y and Z represent C(═O) and R³ represents optionally substituted C₁₋₆alkyl, said intermediates being represented by formula (II-b), can be prepared by reacting an intermediate of formula (XI) with H₂ in the presence of a suitable catalyst, such as for example palladium on charcoal, and a suitable solvent, such as for example an alcohol, e.g. methanol and the like.

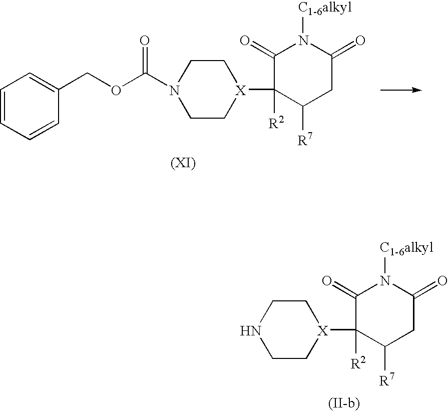

Intermediates of formula (II) wherein Z represents CH₂, Y represents C(═O), X represents CH and R³ represents hydrogen, said intermediates being represented by formula (II-c), can be prepared by reducing an intermediate of formula (XII) in the presence of a suitable reducing agent, such as for example H₂, a suitable catalyst, such as for example palladium on charcoal, and a suitable solvent, such as for example an alcohol, e.g. methanol and the like.

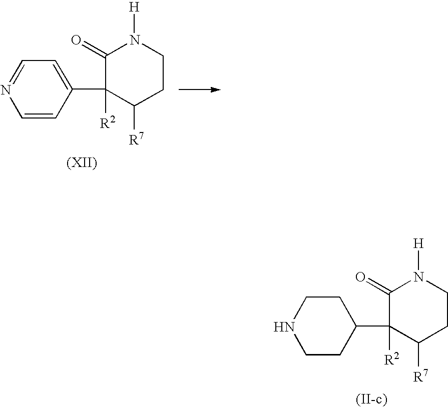

Intermediates of formula (II) wherein Y represents CH₂, Z represents C(═O), X represents CH and R³ represents hydrogen, said intermediates being represented by formula (II-d), can be prepared by reacting an intermediate of formula (XIII) with H₂ in the presence of a suitable catalyst, such as for example palladium on charcoal, in the presence of a suitable solvent, such as for example an alcohol, e.g. methanol and the like. After the initial hydrogenation for debenzylation, the reaction can further be performed using Raney Ni as catalyst for the cyclization. Alternatively, the reaction can also be performed by using Raney Ni as first catalyst for the cyclisation reaction followed by debenzylation with palladium on charcoal as catalyst.

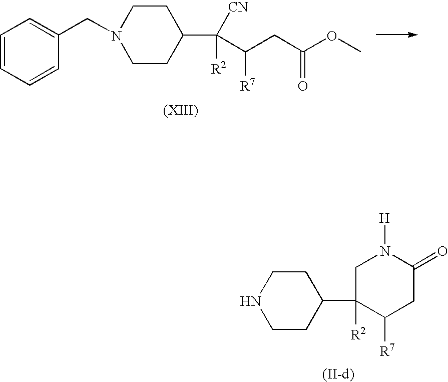

Intermediates of formula (IX) wherein X represents CH, said intermediates being represented by formula (IX-a), can be prepared by reacting an intermediate of formula (XIV) with a suitable acid, such as for example a mixture of H₂SO₄ and acetic acid. When the ring moiety of the R² substituent of an intermediate of formula (XIV) is substituted with C₁₋₆alkyloxycarbonyl, said C₁₋₆alkyloxycarbonyl substituent will be converted into a carboxy substituent during the reaction. This reaction may also result in the introduction of a —SO₂—OH substituent on the ring moiety of R²

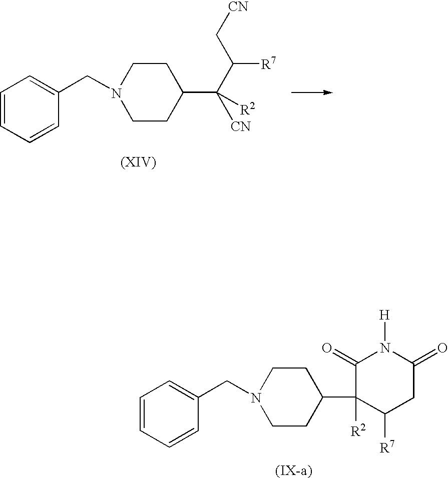

Alternatively, intermediates of formula (IX-a) can also be prepared by reacting an intermediate of formula (XIII) with a suitable acid, such as for example a mixture of H)SO₄ and acetic acid.

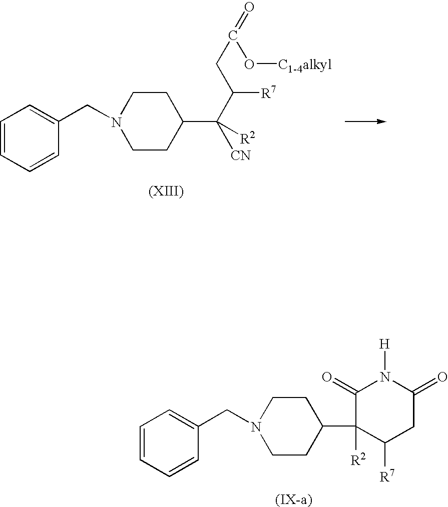

Intermediates of formula (IX) wherein X represents N, said intermediates being represented by formula (IX-b), can be prepared by reacting an intermediate of formula (XV) with a suitable base, such as for example NaOtertBu, in the presence of a suitable solvent, such as for example tetrahydrofuran.

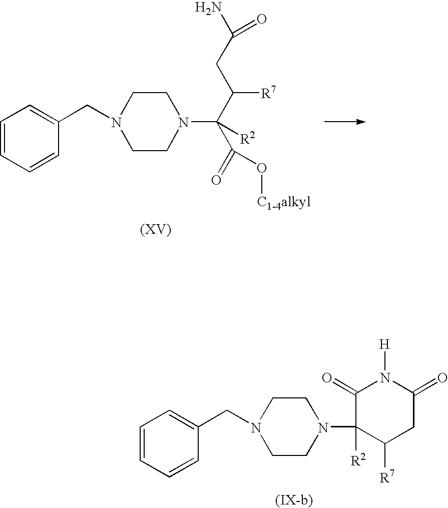

Intermediates of formula (XIII) can be prepared by reacting an intermediate of formula (XVI) with CHR⁷═CH—C(═O)—O—C₁₋₄alkyl in the presence of a suitable catalyst, such as for example TritonB and a suitable solvent, such as for example dioxane. This reaction can also be performed in the presence of a suitable base, such as for example NaOCH₃, and a suitable solvent, such as for example xylene.

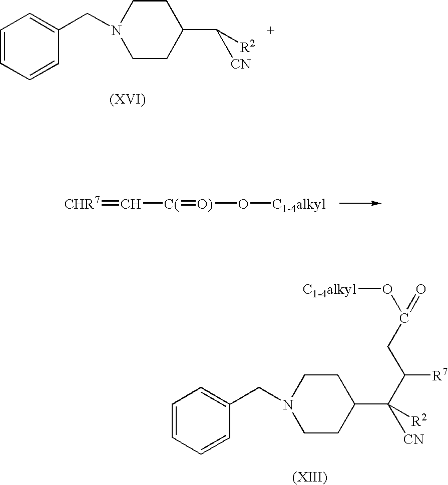

Intermediates of formula (XIV) can be prepared by reacting an intermediate of formula (XVI) with CHR⁷═CH—CN, in the presence of a suitable catalyst, such as for example TritonB, a suitable solvent, such as for example dioxane, and optionally in the presence of a suitable base, such as for example K terbutylate.

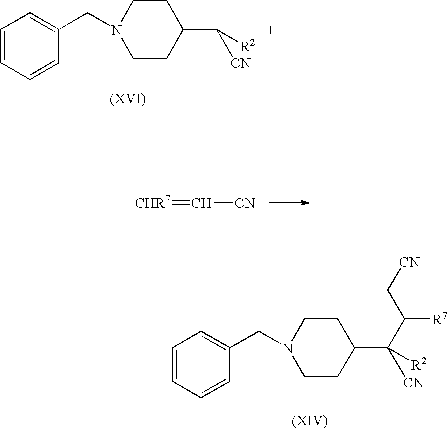

Intermediates of formula (XVI) can be prepared by reducing an intermediate of formula (XVII) with a suitable reducing agent, such as for example H₂, in the presence of a suitable catalyst, such as for example palladium on charcoal or Rhodium on charcoal, optionally a suitable catalyst poison, such as for example a thiophene solution, and a suitable solvent, such as for example an alcohol, e.g. methanol and the like, or tetrahydrofuran. The reaction can also be performed with NaBH₄ as reducing agent in the presence of a suitable solvent, such as for example an alcohol, e.g. 2-propanol and the like.

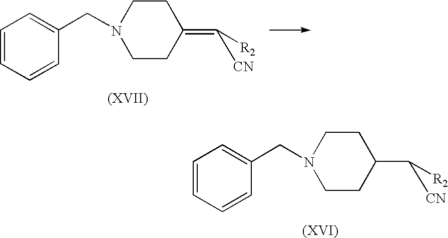

Intermediates of formula (XVII) can be prepared by reacting an intermediate of formula (XVIII) with an intermediate of formula (XIX) in the presence of a suitable base, such as for example NaOCH₃ or Na in CH₃OH, in the presence of a suitable solvent, such as for example an alcohol, e.g. methanol and the like.

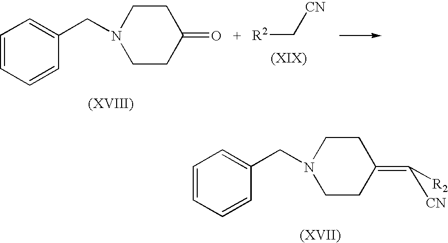

Intermediates of formula (XV) can be prepared by reacting an intermediate of formula (XX) with NH₃ in the presence of a suitable coupling agent, such as for example 1,1′-carbonylbis-1H-imidazole, and in the presence of a suitable solvent, such as for example dichloromethane.

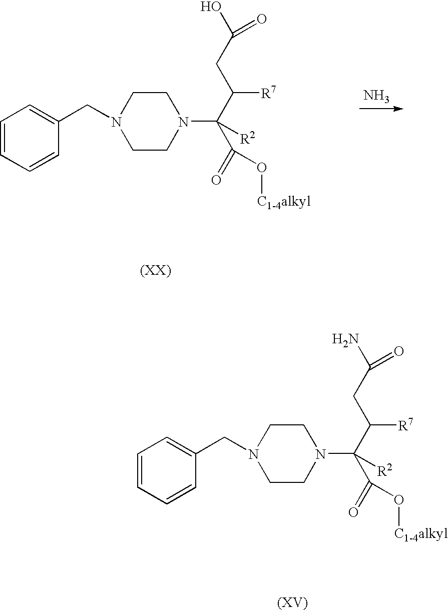

Intermediates of formula (XX) can be prepared by hydrolyzing an intermediate of formula (XXI) with a suitable base, such as for example NaOH, in the presence of a suitable solvent, such as for example dioxane.

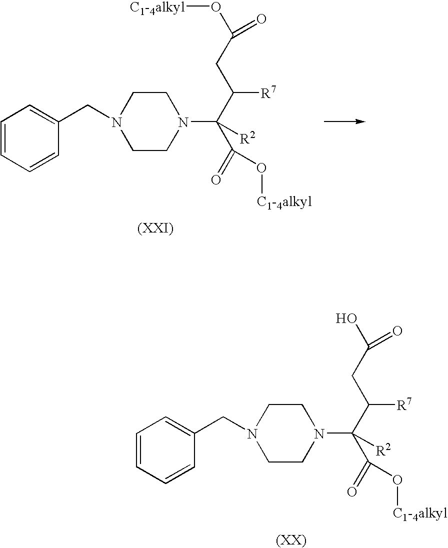

Intermediates of formula (XXI) can be prepared by reacting an intermediate of formula (XXII) with an intermediate of formula (XXIII) wherein W₅ represents a suitable leaving group, such as for example halo, e.g. bromo and the like, in the presence of a suitable base, such as for example NaH and a suitable solvent, such as for example N,N-dimethylformamide.

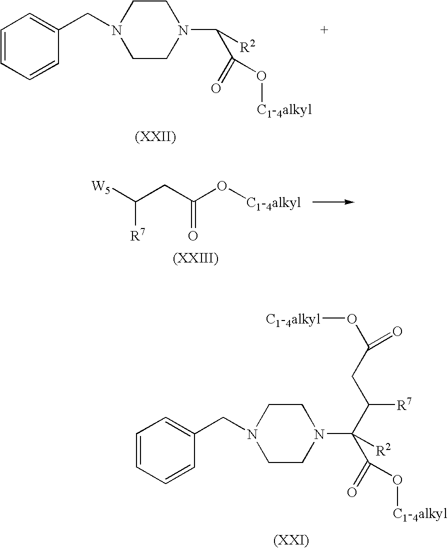

Intermediates of formula (XXII) can be prepared by reacting an intermediate of formula (XXIV) with an intermediate of formula (XXV) wherein W₆ represents a suitable leaving group, such as for example halo, e.g. bromo and the like, in the presence of a suitable base, such as for example N,N-diethylethanamine, and a suitable solvent, such as for example dichloromethane.

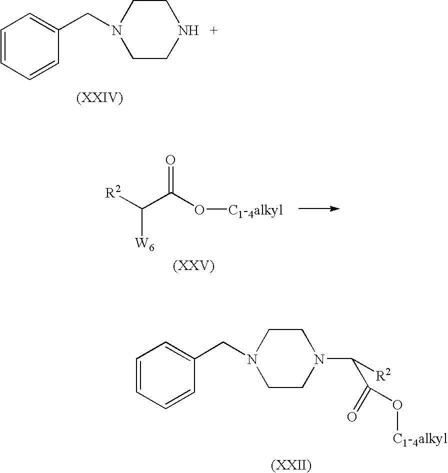

Intermediates of formula (X) can be prepared by reacting an intermediate of formula (XXVI) with a suitable base, such as for example Na tertBuO, in the presence of a suitable solvent, such as for example tetrahydrofuran.

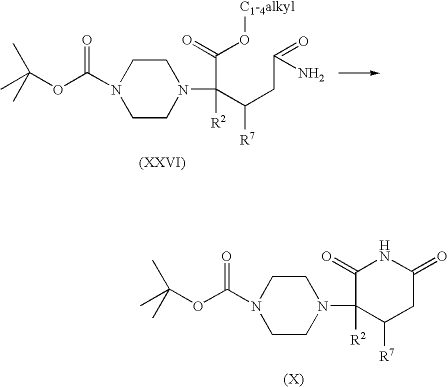

Intermediates of formula (XXVI) can be prepared according to the procedures described for intermediates of formula (XV).

Intermediates of formula (XI) can be prepared by reacting an intermediate of formula (XXVII) with a suitable C₁₋₆alkyl halide, e.g. methyl iodide and the like, in the presence of a suitable base, such as for example K₂CO₃, and a suitable solvent, such as for example N,N-dimethylformamide.

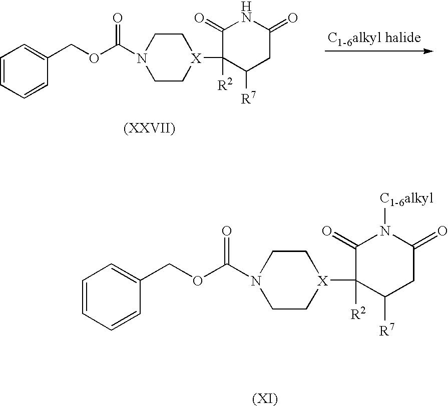

Intermediates of formula (XXVII) can be prepared by reacting an intermediate of formula (II-a) with an intermediate of formula (XXVIII) wherein W₇ represents a suitable leaving group, such as for example halo, e.g. chloro and the like.

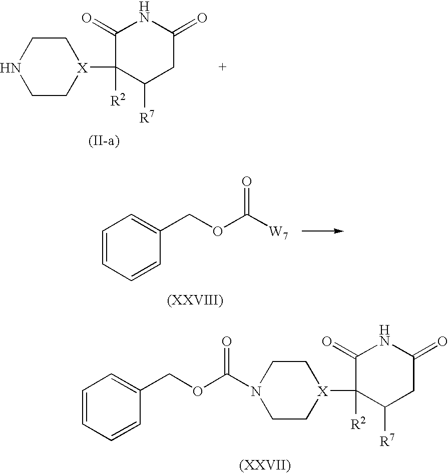

Intermediates of formula (XII) can be prepared by reacting an intermediate of formula (XXIX) with H₂, in the presence of a suitable catalyst, such as for example Raney Ni, and a suitable solvent such as for example tetrahydrofuran.

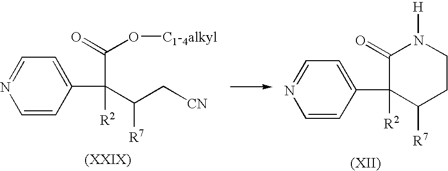

Intermediates of formula (XXIX) can be prepared by reacting an intermediate of formula (XXX) with CHR⁷═CH—CN in the presence of a suitable catalyst, such as for example Triton B, and a suitable solvent, such as for example dioxane.

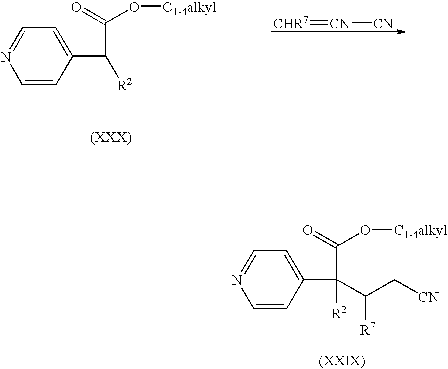

Intermediates of formula (XXX) can be prepared by reacting an intermediate of formula (XXXI) with an intermediate of formula (XXXII) in the presence of a suitable base, such as for example lithium diisopropylamide, and a suitable solvent, such as for example tetrahydrofuran.

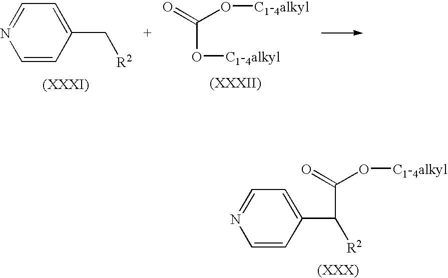

intermediates of formula (IV) can be prepared by reacting an intermediate of formula (XXXIII) with CHR⁷═CH—CN in the presence of a suitable catalyst, such as for example Triton B, and in the presence of a suitable solvent, such as for example dioxane.

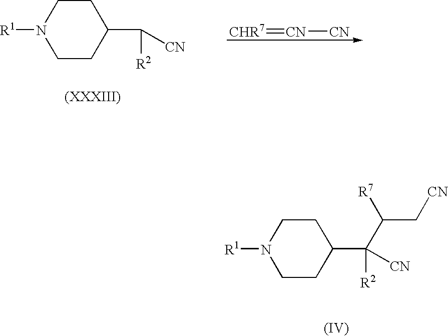

Intermediates of formula (XXXIII) can be prepared by reacting an intermediate of formula (XXXIV) with an intermediate of formula (III) in the presence of a suitable base, such as for example N,N-diisopropylethanamine, and a suitable solvent, such as for example dichloromethane or N,N-dimethylformamide.

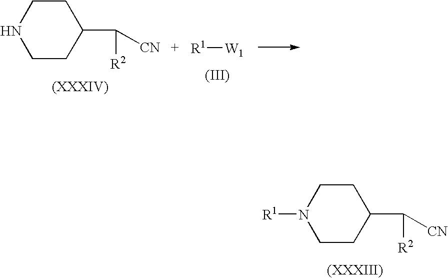

Intermediates of formula (XXXIV) can be prepared by debenzylating an intermediate of formula (XVII) in the presence of H₂, a suitable catalyst, such as for example palladium on charcoal, and a suitable solvent, such as for example an alcohol, e.g. methanol and the like.

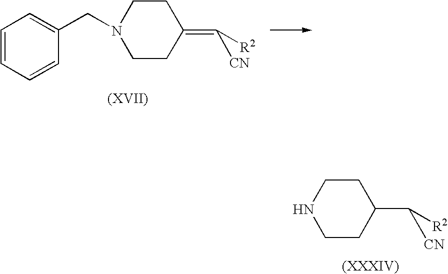

Compounds of formula (I) wherein the ring moiety of the R² substituent is substituted with COOH, said R² substituent being represented as R^{2a}—COOH, and said compounds being represented by formula (I-g), can be converted into an intermediate of formula (VI) wherein W₂ represents chloro, said intermediates being represented by formula (VI-a), by reaction with SOCl₂ in the presence of a suitable solvent, such as for example dichloromethane.

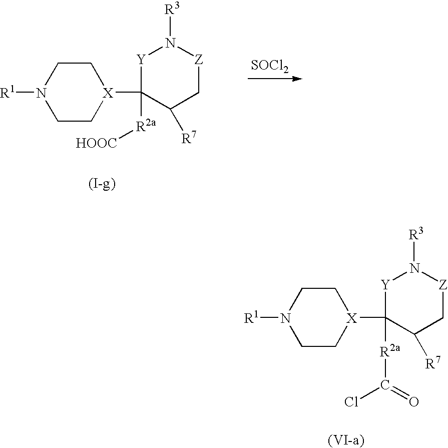

Compounds of formula (I-g) can also be converted into an intermediate of formula (VII) wherein P represents (CH₃)₃C—O—C(═O)—, said intermediate being represented by formula (VII-a), by reaction with phosphorazidic diphenyl ester, 2-methyl-2-propanol and a suitable base, such as for example N,N-diethylethanamine (Curtius rearrangement).

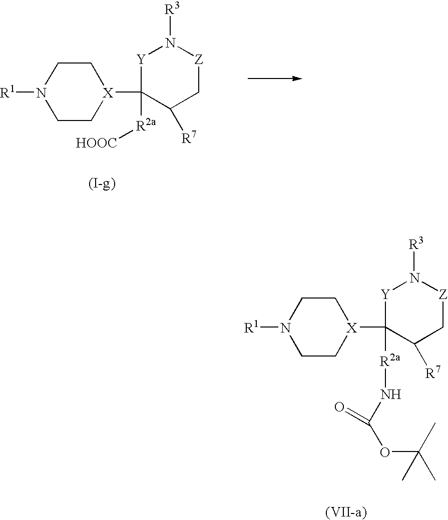

### Pharmacological Part

The compounds of formula (J) and any subgroup thereof show CXCR3 receptor antagonistic properties. Such CXCR3 antagonists can inhibit binding of one or more chemokines (e.g., CXC-chemokines, such as IP-10, MIG and/or I-TAC) to CXCR3 receptor.

Chemokines (contraction of “chemotactic cytokines”) are most important regulators of leukocyte trafficking. This biological role is exerted by interacting—on target cells—with seven-transmembrane-domain receptors that are coupled to heterodimeric G proteins. Chemokines are mainly grouped into 4 major families (C—C; C—X—C; C and C—X₃—C family) dependent on whether the two conserved cysteine residues (represented by C) near the amino terminus are separated by a single amino acid (represented by X) (C—X—C), are adjacent (C—C), have a missing cysteine pair (C) or are separated by three amino acids (C—X₃—C).

The CXCR3 chemokine receptor is a G protein coupled receptor also known as CD183. The CXCR3 receptor is mainly expressed on activated or stimulated T lymphocytes, Natural Killer cells (NK cells), malignant B lymphocytes, endothelial cells, thymocytes and plasma cells. The selective expression of the CXCR3 receptor makes it a suitable target for intervention to interrupt inappropriate T cell trafficking.

Ligands which act through the CXCR3 receptor are the CXC chemokines I-TAC (interferon-inducible T cell alpha-chemoattractant), IP-10 (interferon-inducible protein 10) and MIG (monokine induced by gamma-interferon); I-TAC having the highest receptor affinity.

Clinical indications for intervening in, in particular inhibiting, inappropriate T-cell trafficking via interaction with the CXCR3 receptor are:
(1) inflammatory or allergic diseases such as systemic anaphylaxis or hypersensitivity responses, drug allergies (e.g. to penicillin, cephalosporins), insect sting allergies; inflammatory bowel diseases, such as Crohn's disease, colitis (e.g. ulcerative colitis), ileitis and enteritis; vaginitis; psoriasis and inflammatory dermatoses such as dermatitis, eczema, atopic dermatitis, allergic contact dermatitis, urticaria; vasculitis (e.g. necrotizing, cutaneous, and hypersensitivity vasculitis); spondyloarthropathies; scleroderma; respiratory allergic diseases such as asthma, allergic rhinitis, obstructive pulmonary disease (COPD), hypersensitivity lung diseases, hypersensitivity pneumonitis, interstitial lung diseases (ILD) (e.g. idiopathic pulmonary fibrosis, or ILD associated with rheumatoid arthritis, or other autoimmune conditions), idiopathic pneumonia; and the like,
(2) autoimmune diseases, such as arthritis (e.g. rheumatoid arthritis, psoriatic arthritis, juvenile rheumatoid arthritis, polyarthritis, spondyloarthropathy), multiple sclerosis, systemic lupus erythematosus, myasthenia gravis, diabetes (including diabetes mellitus and juvenile onset diabetes), Sjogren's syndrome, glomerulonephritis and other nephritides, autoimmune thyroid disorders, such as e.g. thyroiditis, and the like;
(3) graft rejection (including allograft rejection (e.g. cardiac, renal and lung rejection), xenograft rejection and graft-v-host disease), and
(4) other diseases in which undesired inflammatory responses are to be inhibited (e.g. atherosclerosis, restenosis, cytokine-induced toxicity, myositis (including polymyositis, dermatomyositis), neurodegenerative diseases, Alzheimer's disease, encephalitis, meningitis, hepatitis, nephritis, sepsis, sarcoidosis, conjunctivitis, otitis, retinopathy (e.g. retinopathy of prematurity, diabetic retinopathy), retinal vein occlusion, macular degeneration (e.g. age-related macular degeneration), hemangiomas, chronic obstructive pulmonary disease, sinusitis and Behcet's syndrome.

Reference therefore is made to Arimili et al, Immunological Reviews, 2000, vol 177, 43-51; Xanthou et al., Eur. J. Immunol., 2003, vol 33, 2927-2936; WO 01/16114 and WO 02/85861; which are incorporated herein by reference.

Due to their CXCR3 receptor antagonistic activity, the compounds of formula (I), their N-oxides, pharmaceutically acceptable salts, stereochemically isomeric forms or solvates are useful for the treatment or prevention, in particular for the treatment, of a disease or condition mediated through the activation of the CXCR3 receptor.

In view of the above-described pharmacological properties, the compounds of formula (I), their N-oxides, pharmaceutically acceptable salts, stereochemically isomeric forms and solvates, may be used as a medicine. In particular, the present compounds can be used for the manufacture of a medicament for treating or preventing a disease mediated through activation of the CXCR3 receptor, in particular for treating a disease mediated through activation of the CXCR3 receptor. More in particular, the compounds of the invention can be used for the manufacture of a medicament for treating or preventing, preferably treating, a CXCR3 mediated inflammatory or allergic disease, a CXCR3 mediated autoimmune disease, a CXCR3 mediated graft rejection, other CXCR3 mediated diseases in which undesired inflammatory responses are to be inhibited. Even more in particular, the compounds of the invention can be used for the manufacture of a medicament for treating or preventing (1) inflammatory or allergic diseases such as systemic anaphylaxis or hypersensitivity responses, drug allergies (e.g. to penicillin, cephalosporins), insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis (e.g. necrotizing, cutaneous, and hypersensitivity vasculitis); spondyloarthropathies; scleroderma; respiratory allergic diseases such as asthma, allergic rhinitis, obstructive pulmonary disease (COPD), hypersensitivity lung diseases, hypersensitivity pneumonitis, interstitial lung diseases (ILD) (e.g. idiopathic pulmonary fibrosis, or ILD associated with rheumatoid arthritis, or other autoimmune conditions), idiopathic pneumonia; and the like; (2) autoimmune diseases, such as arthritis (e.g. rheumatoid arthritis, psoriatic arthritis, juvenile rheumatoid arthritis, polyarthritis, spondyloarthropathy), multiple sclerosis, systemic lupus erythematosus, myasthenia gravis, diabetes (including diabetes mellitus and juvenile onset diabetes), Sjogren's syndrome, glomerulonephritis and other nephritides, autoimmune thyroid disorders, such as e.g. thyroiditis, and the like; (3) graft rejection (including allograft rejection (e.g. cardiac, renal and lung rejection), xenograft rejection and graft-v-host disease), and (4) other diseases in which undesired inflammatory responses are to be inhibited (e.g. atherosclerosis, restenosis, cytokine-induced toxicity, myositis (including polymyositis, dermatomyositis), neurodegenerative diseases, Alzheimer's disease, encephalitis, meningitis, hepatitis, nephritis, sepsis, sarcoidosis, conjunctivitis, otitis, retinopathy (e.g. retinopathy of prematurity, diabetic retinopathy), retinal vein occlusion, macular degeneration (e.g. age-related macular degeneration), hemangiomas, chronic obstructive pulmonary disease, sinusitis and Behcet's syndrome. Further, the compounds of the invention can be used for the manufacture of a medicament for treating or preventing rheumatoid arthritis, inflammatory bowel diseases such as Crohn's disease and colitis, allograft rejection (e.g. cardiac, renal, lung allograft rejection), multiple sclerosis, COPD, glomerulonephritis, allergic contact dermatitis, lupus, psoriasis, atherosclerosis, Sjogren's syndrome, autoimmune thyroid disorders. Preferably, the present compounds can be used for treating or preventing, especially treating, rheumatoid arthritis, inflammatory bowel diseases such as Crohn's disease and colitis, allograft rejection (e.g. cardiac, renal, lung allograft rejection).

In view of the utility of the compounds of formula (I), there is provided a method of treating a warm-blooded mammal, including a human, suffering from or a method of preventing a warm-blooded mammal, including a human, to suffer from a disease mediated through activation of the CXCR3 receptor, in particular a method of treating a warm-blooded mammal, including a human, suffering from a disease mediated through activation of the CXCR3 receptor. Said methods comprise the administration of an effective amount of a compound of formula (I), a N-oxide form thereof, a pharmaceutically acceptable salt thereof, a possible stereoisomeric form thereof or a solvate thereof, to a warm-blooded mammal, including a human.

The present invention also provides compositions for preventing or treating a disease mediated through activation of the CXCR3 receptor, in particular for treating a disease mediated through activation of the CXCR3 receptor. Said compositions comprise a therapeutically effective amount of a compound of formula (I), a N-oxide form thereof, a pharmaceutically acceptable salt thereof, a stereoisomeric form thereof or a solvate thereof, and a pharmaceutically acceptable carrier or diluent.

The compounds of the present invention may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in salt form, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, diluents, lubricants, binders, disintegrating agents and the like in the case of powders, pills, capsules, and tablets. Because of their ease in administration, tablets and capsules represent the most advantageous oral dosage unit forms, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations, which are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

The compounds of the present invention may also be administered via inhalation or insufflation by means of methods and formulations employed in the art for administration via this way. Thus, in general the compounds of the present invention may be administered to the lungs in the form of a solution, a suspension or a dry powder. Any system developed for the delivery of solutions, suspensions or dry powders via oral or nasal inhalation or insufflation are suitable for the administration of the present compounds.

The compounds of the present invention may also be topically administered in the form of drops, in particular eye drops. Said eye drops may be in the form of a solution or a suspension. Any system developed for the delivery of solutions or suspensions as eye drops are suitable for the administration of the present compounds.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

The exact dosage and frequency of administration depends on the particular compound of formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will preferably comprise from 0.05 to 99% by weight, more preferably from 0.1 to 70% by weight, even more preferably from 0.1 to 50% by weight of the active ingredient, and, from 1 to 99.95% by weight, more preferably from 30 to 99.9% by weight, even more preferably from 50 to 99.9% by weight of a pharmaceutically acceptable carrier, all percentages being based on the total weight of the composition.

The compounds of formula (I) may also be used in combination with other conventional anti-inflammatory or immunosuppressive agents, such as steroids, cyclooxygenase-2 inhibitors, non-steroidal-anti-inflammatory drugs, TNF-α antibodies, such as for example acetyl salicylic acid, bufexamac, diclofenac potassium, sulindac, diclofenac sodium, ketorolac trometamol, tolmetine, ibuprofen, naproxen, naproxen sodium, tiaprofen acid, flurbiprofen, mefenamic acid, nifluminic acid, meclofenamate, indomethacin, proglumetacine, ketoprofen, nabumetone, paracetamol, piroxicam, tenoxicam, nimesulide, fenylbutazon, tramadol, beclomethasone dipropionate, betamethasone, beclamethasone, budesonide, fluticasone, mometasone, dexamethasone, hydrocortisone, methylprednisolone, prednisolone, prednisone, triamcinolone, celecoxib, rofecoxib, valdecoxib, infliximab, leflunomide, etanercept, CPH 82, methotrexate, sulfasalazine, antilymphocytory immunoglobulines, antithymocytory immunoglobulines, azathioprine, cyclosporine, tacrolimus substances, ascomycin, rapamycin, muromonab-CD3.

Thus, the present invention also relates to the combination of a compound of formula (I) and another anti-inflammatory or immunosuppressive agent. Said combination may be used as a medicine. The present invention also relates to a product containing (a) a compound of formula (I), and (b) another anti-inflammatory or immunosuppressive compound, as a combined preparation for simultaneous, separate or sequential use in the treatment of a disease mediated through activation of the CXCR3 receptor. The different drugs in such products may be combined in a single preparation together with pharmaceutically acceptable carriers. Alternatively, such products may comprise, for example, a kit comprising a container with a suitable composition containing a compound of formula (I) and another container with a composition containing another anti-inflammatory or immunosuppressive compound. Such a product may have the advantage that a physician can select on the basis of the diagnosis of the patient to be treated the appropriate amounts of each component and the sequence and timing of the administration thereof.

The following examples are intended to illustrate the present invention.

### EXPERIMENTAL PART

Hereinafter “THF” means tetrahydrofuran, “DIPE” means diisopropylether, “Triton-B” means N,N,N-trimethylbenzenemethanaminium hydroxide, “DMF” means N,N-dimethylformamide and “DCM” means dichloromethane.

A number of compounds were purified by reversed phase high-performance liquid chromatography using the method below (indicated in the procedure with method A).

### HPLC Method A

The product was purified by high-performance liquid chromatography (RP18 BDS 8 μm 250 g; I.D. 5 cm). Three mobile phases (mobile phase A: a 0.25% NH₄HCO₃ solution; mobile phase B: CH₃OH; mobile phase C: CH₃CN) were employed. First, 75% A and 25% B with a flow rate of 40 ml/min was hold for 0.5 minutes. Then a gradient was applied to 50% B and 50% C in 41 minutes with a flow rate of 80 ml/min. Then a gradient was applied to 100% C in 20 minutes with a flow rate of 80 ml/min and hold for 4 minutes.

### A. Preparation of the Intermediate Compounds

### Example A1

### a) Preparation of Intermediate 1

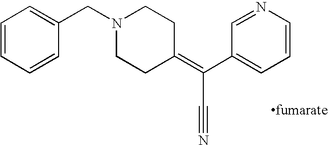

A solution of 3-pyridineacetonitrile (0.117 mol) in CH₃OH p.a. (150 ml) was stirred under N₂ atmosphere. CH₃ONa 30% solution in CH₃OH (5.5 M) (0.12 mol) and 1-(phenylmethyl)-4-piperidinone (0.0588 mol) were added and the reaction mixture was stirred and refluxed for 18 hours. Then the mixture was cooled to room temperature, poured into ice-water (300 ml) and the product was extracted 2× with DCM. The separated organic layer was dried (MgSO₄), filtered, and the solvent was evaporated. The residue was purified by column chromatography over silica gel (eluent: EtOAc/hexane 60/40). The product fractions were collected and the solvent was evaporated. The residue was crystallized as its fumarate (5 g added) from 2-propanol. Yield: 12.1 g of intermediate 1 (50.7%).

### b) Preparation of Intermediate 2

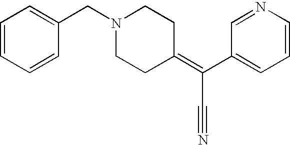

A mixture of intermediate 1 (0.0296 mol) and K₂CO₃ (aqueous saturated solution) (125 ml) in EtOAc (125 ml) was stirred vigorously for 3 hours. The organic layer was separated and washed with H₂O, dried (MgSO₄), filtered and the solvent was evaporated. Yield: 8.87 g of intermediate 2.

### c) Preparation of Intermediate 3

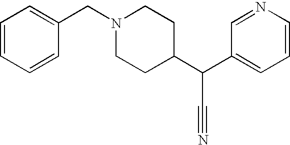

A solution of intermediate 2 (0.029 mol) in CH₃OH p.a. (150 ml) was hydrogenated at 50° C. with Pd/C 10% (1 g) as a catalyst in the presence of thiophene solution in CH₃OH (0.5 ml). After uptake of H₂ (1 equiv), the catalyst was filtered off and the filtrate was evaporated, yielding intermediate 3 used as such in the next step.

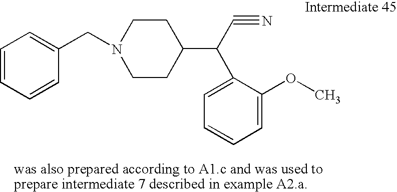

### d) Preparation of Intermediate 4

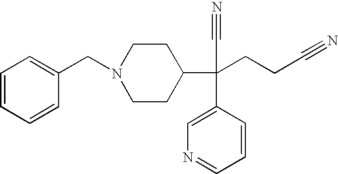

A solution of intermediate 3 (0.029 mol) in 2-propenenitrile (2.3 ml) and 1,4-dioxane (p.a.; dried on molecular sieves; 100 ml) was stirred under N₂ atmosphere on an ice-bath. When stirring became difficult, Triton-B (0.5 ml) was added and the reaction mixture was stirred further without cooling for 18 hours. The solvent was evaporated, yielding intermediate 4 used as such in the next step.

### e) Preparation of Intermediate 5

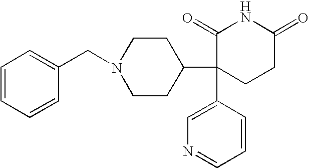

Intermediate 4 (0.029 mol) was dissolved in acetic acid (80 ml). H₂SO₄ (20 ml) was added slowly to the solution. The reaction mixture was put in a pre-heated oil-bath (165° C.), and then stirred and refluxed for 3.5 hours. The mixture was allowed to cool to room temperature, then poured out into a mixture of ice/ice-water, containing 70 ml of a 50% aqueous NaOH solution. This mixture was extracted with DCM/CH₃OH 90/10. The layers were separated. A saturated aqueous K₂CO₃ solution was added to the water layer in order to bring the pH to value 7. This mixture was extracted again with DCM/CH₃OH 90/10. The combined organic layers were dried (MgSO₄), filtered and the solvent was evaporated. Toluene was added again and co-evaporated on the rotary evaporator. The residue was purified over silica gel on a glass filter (eluent: DCM/CH₃OH 95/5). The desired fractions were collected and the solvent was evaporated. Toluene was added and co-evaporated on the rotary evaporator. The residue was stirred in DIPE and the resulting precipitate was filtered off, washed with DIPE and dried (vacuum, 55° C.). Yield: 6.2 g of intermediate 5 (58.8%).

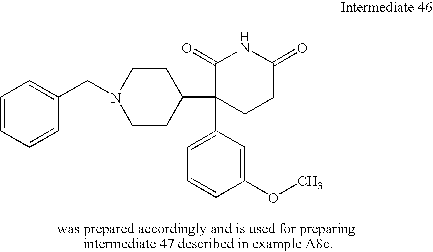

### f) Preparation of Intermediate 6

A solution of intermediate 5 (0.0168 mol) in CH₃OH p.a. (200 ml) was hydrogenated at 50° C. with Pd/C 10% (1 g) as a catalyst. After uptake of H₂ (1 equiv), the catalyst was filtered off and the filtrate was evaporated. The remaining solid was triturated in Et₂O, filtered off, washed with Et₂O and dried (vacuum, 55° C.). Yield: 4.48 g of intermediate 6 (97.5%).

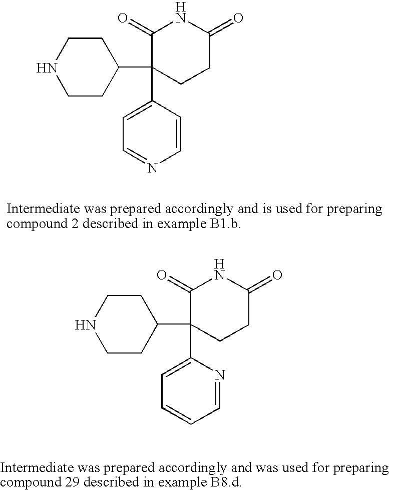

### Example A1A

### a) Preparation of Intermediate 48

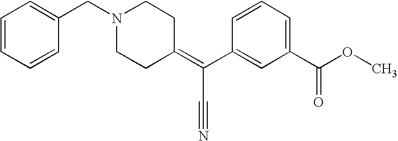

Reaction under N₂ atmosphere. A solution of 3-(cyanomethyl)benzoic acid methyl ester (0.0314 mol) in CH₃OH, p.a. (60 ml) was stirred at room temperature. NaOMe, 30% in CH₃OH (0.032 mol) was added. 1-(phenylmethyl)-4-piperidone (0.0157 mol) was added and the reaction mixture was stirred and refluxed for 18 hours. The mixture was allowed to cool to room temperature. The mixture was poured out into ice-water (180 ml). The product was extracted with DCM (2×). The combined organic layers were dried (MgSO₄), filtered and the solvent was evaporated. The residue (7 g) was purified over silica gel on a glass filter (eluent: DCM/CH₃OH 99.5/0.5). The product fractions were collected and the solvent was evaporated. EtOAc was added and co-evaporated. Yield: 1.38 g of intermediate 48.

### b) Preparation of Intermediate 49

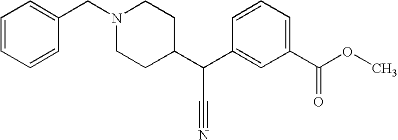

A solution of intermediate 48 (0.04 mol) in CH₃OH (250 ml) was hydrogenated with Pd/C 10% (3 g) as a catalyst in the presence of thiophene solution in CH₃OH (1 ml). After uptake of H₂ (1 equiv), the catalyst was filtered off and the filtrate was evaporated and co-evaporated with 1,4-dioxane, yielding intermediate 49 used as such in the next step.

### c) Preparation of Intermediate 50

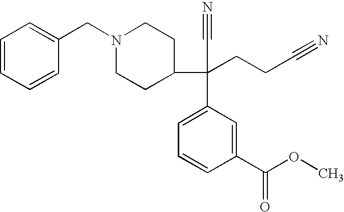

A solution of intermediate 49 (0.04 mol) and 2-propenenitrile (3.2 ml) in 1,4-dioxane (p.a.; dried on molecular sieves; 150 ml) was stirred under N₂ flow on an ice-bath. When stirring became difficult, Triton-B (catalyst) (0.5 ml) was added and the reaction mixture was further stirred on the ice-bath for 2 minutes and at room temperature for 20 hours. The solvent was evaporated, yielding intermediate 50 used as such in the next step.

### d) Preparation of Intermediate 51

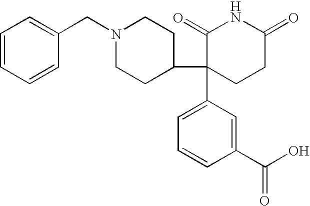

A solution of intermediate 50 (0.04 mol) in acetic acid (75 ml) was stirred. H₂SO₄ (20 ml) was added and the reaction mixture was stirred and refluxed in a pre-heated oil bath at 165° C. for 150 minutes. The reaction mixture was allowed to reach ±60° C. and poured out slowly into ice-water containing NaOH (85 ml, 50%). Stirring was continued for 1 hour. The solid part was filtered off, washed with H₂O and dried (vacuum with airstream, 50° C.). Yield: 13.5 g of fraction A (reaction product mainly as —COOH). A DCM/CH₃OH 90/10 solution was added to the filtrate, and the biphasic solution was stirred vigorously for 1 hour. The separated organic layer was dried (MgSO₄), filtered and the solvent was evaporated. Yield: 1.3 g of fraction B (reaction product mainly as —COOCH₃). The separated water layer was neutralized with HCl (1 N) till pH=7. The formed precipitate was filtered off, combined with fraction A and purified by reversed-phase high-performance liquid chromatography (NH₄OAc). The product fractions were combined and the solvent was evaporated. The residue was stirred in boiling H₂O (125 ml), filtered off at room temperature and the solid was dried (vacuum, 55° C.). Yield: 6.7 g of intermediate 51 (41.2%).

Fraction B was purified by reversed-phase high-performance liquid chromatography (NH₄OAc). The product fractions were combined and the solvent was evaporated. The residue was stirred in H₂O (20 ml) and the product was extracted with DCM/CH₃OH 95/5. The separated organic layer was dried (MgSO₄), filtered and the solvent was evaporated and co-evaporated with toluene. Yield: 0.54 g of fraction C (reaction product as —COOCH₃)

### e) Preparation of Intermediate 52

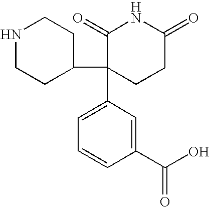

A solution of intermediate 51 (0.016 mol) in acetic acid (250 ml) was hydrogenated with Pd/C 10% (2 g) as a catalyst. After uptake of H₂ (1 equiv), the catalyst was filtered off and the filtrate was evaporated at 40° C. and two times co-evaporated with toluene. The remaining oil was triturated in Et₂O, filtered off, washed and dried (vacuum, 50° C.). Yield: 5.5 g of intermediate 52 (100%).

### Example A2

### a) Preparation of Intermediate 7

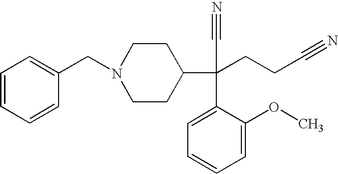

Reaction under N₂ atmosphere. A solution of intermediate 45

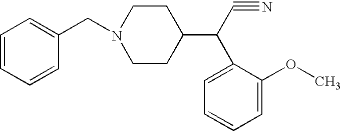

(prepared according to A1.c) (0.029 mol) in 2-propenenitrile (2.3 ml, 0.0348 mol) and 1,4-dioxane (100 ml) was stirred and cooled on an ice-bath. When stirring became difficult, Triton-B (0.2 ml) was added and the reaction mixture was stirred for 20 minutes while cooling on the melting ice-bath. Then, the reaction mixture was stirred for 18 hours at room temperature. More Triton-B (0.15 ml) was added and the mixture was stirred for 24 hours at room temperature. More Triton-B (1 ml) was added. Extra 2-propenenitrile (1 ml) was added and the reaction mixture was stirred over the weekend at room temperature, then for 20 hours at 50° C. Extra 2-propenenitrile (1.3 ml) and Triton-B (0.25 ml) were added and the reaction mixture was stirred for 20 hours at 50° C. Again, 2-propenenitrile (5 ml) and Triton-B (0.2 ml) were added. The mixture was stirred for 20 hours at 50° C. CH₃ONa (1.8 g) was added at room temperature and the reaction mixture was stirred for 5 hours at room temperature, then for 18 hours at 50° C., then it stood over the weekend. More CH₃ONa (1.8 g) was added. Extra 2-propenenitrile (5 ml) was added and the reaction mixture was stirred for 20 hours at 50° C. The mixture was allowed to cool to room temperature, then it was poured out into ice-water (500 ml). EtOAc (300 ml) was added and the biphasic mixture was stirred for one hour. The biphasic mixture was filtered through dicalite. The filtrate's organic phase was separated, dried (MgSO₄), filtered and the solvent was evaporated. The residue was purified over silica gel on a glass filter (eluent: DCM/CH₃OH 98/2). The desired fractions were collected and the solvent was evaporated. 1,4-Dioxane was added, then co-evaporated again. Yield: 11.7 g of intermediate 7 (mixture of starting material A and target compound: 70/30).

### b) Preparation of Intermediate 8

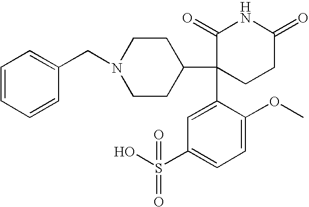

H₂SO₄ (20 ml) was added slowly to a mixture of intermediate 7 (11.7 g) in acetic acid, p.a. (80 ml), stirred at room temperature. The reaction mixture was stirred and refluxed for 4.5 hours in an oil-bath (oil-bath temperature: 165° C.). The mixture was allowed to cool to ±40° C. Then, it was poured out slowly into a stirring mixture of ice and ice-water, containing 50% NaOH (75 ml). DCM/CH₃OH 95/5 (300 ml) was added. Then, the mixture was neutralized (pH=7) by gently adding a saturated aqueous K₂CO₃ solution. Stirring was continued overnight. The solid part was filtered off. The solid was washed with water, washed with DCM, washed with water, again with DCM, then dried (vacuum, 55° C., gentle stream of air). Yield: 4.3 g. This fraction was purified by high-performance liquid chromatography over RP-18 (eluent: (0.5% NH₄OAc in H₂O)/CH₃CN/CH₃OH gradient). The desired fractions were collected and the organic solvents were evaporated. The remaining aqueous phase was concentrated to a 250 ml volume. The concentrate stood overnight. The resultant precipitate was filtered off, washed with water and dried (vacuum, 55° C.). Yield: 1.98 g of intermediate 8.

### c) Preparation of Intermediate 9

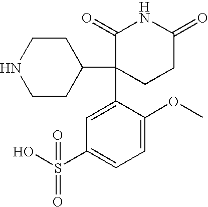

A solution of intermediate 8 (0.00211 mol) in CH₃OH, p.a. (50 ml) was hydrogenated with Pd/C 10% (0.5 g) as a catalyst. After uptake of H₂ (1 equivalent), the catalyst was filtered off, rinsed with a lot of water, and the filtrate was evaporated. The residue was stirred in ethanol, filtered off, washed with ethanol, and dried (vacuum, 55° C.). Yield: 0.45 g of intermediate 9.

### Example A3

### a) Preparation of Intermediate 10

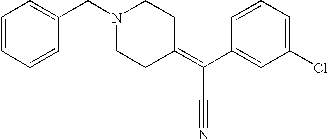

A solution of 3-chloro-benzeneacetonitrile (0.094 mol) in CH₃OH p.a. (175 ml) was stirred under N₂ atmosphere. CH₃ONa (30% in CH₃OH; 5.5 M) (0.094 mol; 17.1 ml) and 1-(phenylmethyl)-4-piperidinone (0.047 mol) were added and the reaction mixture was stirred and refluxed for 18 hours. Then the mixture was cooled to room temperature, poured into ice-water (250 ml) and the product was extracted 2× with DCM. The separated organic layer was dried (MgSO₄), filtered, and the solvent was evaporated and co-evaporated with DIPE. The residue was purified over silica gel on a glass filter (eluent: DCM/CH₃OH 99/1). The product fractions were collected and the solvent was evaporated and co-evaporated with toluene. Yield: 12.7 g of intermediate 10 (83.7%).

### b) Preparation of Intermediate 11

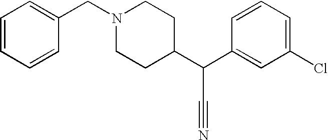

A solution of intermediate 10 (0.037 mol) in CH₃OH (150 ml) was hydrogenated at 50° C. with Rh/C₅% (2 g) as a catalyst in the presence of thiophene solution in CH₃OH (0.5 ml). After uptake of H₂ (1 equiv), the catalyst was filtered off and the filtrate was evaporated. Yield: 12.2 g of intermediate 11

### c) Preparation of Intermediate 12

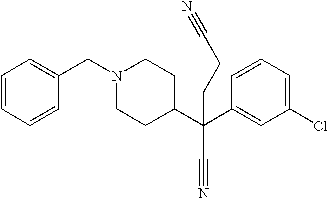

Reaction under N₂ atmospheres. A solution of intermediate 11 (0.037 mol) in 2-propenenitrile (3 ml, 0.045 mol) and 1,4-dioxane (dried on moleculare sieves; 125 ml) was stirred and cooled on an ice-bath. When stirring became difficult, Triton-B (0.25 ml) was added and the reaction mixture was stirred for 30 minutes under moderate cooling. Then, the reaction mixture was stirred for 18 hours at room temperature. More Triton-B (0.5 ml) was added and the reaction mixture was stirred for 20 hours at room temperature (on a water-bath). The mixture stood for 3 days. The solvent was evaporated, yielding intermediate 12 used as such in the next step.

### d) Preparation of Intermediate 13

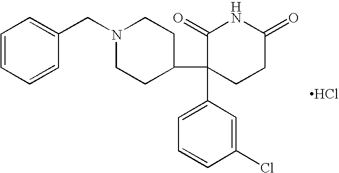

H₂SO₄ (20 ml) was added slowly to a solution of intermediate 12 (0.037 mol) in CH₃COOH (80 ml). The reaction mixture was stirred and refluxed for 210 minutes at 165° C. and then stirred overnight at room temperature. The mixture was poured into a stirring ice-water mixture containing a saturated aqueous NaOH solution (70 ml). The product was extracted 2× with DCM. The separated organic layer was dried (MgSO₄), filtered and the solvent was evaporated and co-evaporated with toluene. The residue was purified over silica gel on a glass filter (eluent: DCM/CH₃OH 97/3). The product fractions were collected and the solvent was evaporated and co-evaporated with toluene. The remaining foam (8.3 g) was turned into its HCl-salt in hot 2-propanol (50 ml) by addition of HCl/2-propanol (10 ml, 6N). Immediate precipitation occurred and the product was filtered off hot, washed with hot 2-propanol and DIPE, and dried (vacuum, 55° C.). Yield: 7.8 g of intermediate 13.

### e) Preparation of Intermediate 14

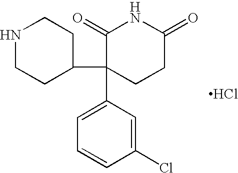

A solution of intermediate 13 (0.0055 mol) and N-ethyl-N-(1-methylethyl)-2-propanamine (0.0055 mol) in dichloroethane (25 ml) was stirred under N₂ atmosphere at room temperature. 1-Chloroethylcarbonochloridic acid ester (0.0165 mol) was added. The reaction mixture was stirred and refluxed for 18 hours. The mixture was allowed to cool to room temperature. The reaction was quenched by addition of CH₃OH, p.a. (10 ml). The reaction mixture was stirred and refluxed for 40 minutes, the mixture was allowed to cool to room temperature, then stood for 5 hours at room temperature. The precipitate was filtered off, washed with toluene/CH₃OH 2/1, then with DIPE, and dried (vacuum, 55° C.). Yield: 1.27 g of intermediate 14 (67.3%).

### Example A4

### a) Preparation of Intermediate 15

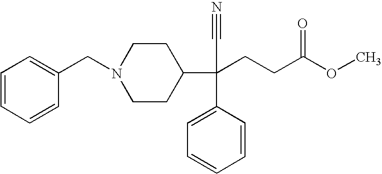

Reaction under N₂ atmosphere. Dimethylbenzene (206 L) was poured into a 500-L RVS reactor, previously flushed with N₂ gas. α-Phenyl-1-(phenylmethyl)-4-piperidineacetonitrile (60 kg) was added and this mixture was heated to reflux temperature. The solution was distilled azeotropically using a water separator, until it was free of water. N₂ gas was let in. The mixture was cooled to ±65° C. CH₃ONa 30% (9.3 kg, 51.6 mol) was added dropwise at ±65° C. 2-Propenoic acid methyl ester (26.7 kg, 310 mol) was added dropwise at ±65° C. over a 30-minutes period. The addition container was flushed with dimethylbenzene (20 L). The reaction mixture was stirred for 4-6 hours at ±70° C., then it was cooled to room temperature. NaCl (5.2 kg) and water (133 L) were added and the mixture was stirred for at least 15 minutes. The layers were allowed to separate slowly. The organic layer was separated, treated with NaCl (2.6 kg) in water (67 L) and stirred for at least 15 minutes. The layers were allowed to separate slowly. The organic layer was separated, dried (Na₂SO₄, 5 kg), filtered over a cotton bag, and the filtrate's solvent was evaporated (in vacuo; container temperature: 100° C.). Yield: 71.7 kg of intermediate 15 (92%).

### b) Preparation of Intermediate 16

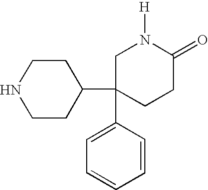

Intermediate 15 (max 0.01 mol) was dissolved in CH₃OH (200 ml) and this solution was hydrogenated at room temperature with Pd/C 10% (q.s.) as a catalyst. After uptake of H₂ (1 equiv), the catalyst was filtered off and the filtrate (containing debenzylated starting material intermediate 15) was hydrogenated further at room temperature with Raney Nickel (1 g) as a catalyst. After uptake of H₂ (2 equiv), the catalyst was filtered off and the filtrate was evaporated. Toluene was added and co-evaporated on the rotary evaporator. Methanol was added, then co-evaporated. Yield: 1.45 g of intermediate 16 (56.1%).

### Example A4A

### a. Preparation of Intermediate 56

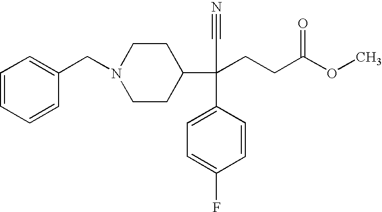

A solution of 1-benzyl-α-(p-fluorophenyl)-4-piperidineacetonitrile (0.0233 mol) in 2-propenoic acid methyl ester (2.5 ml) and 1,4-dioxane (p.a.; dried over molecular sieves; 80 ml) was stirred under N₂ atmosphere while cooling on an ice-bath. When stirring became difficult, Triton-B (0.3 ml) was added and the resultant reaction mixture was stirred for 10 minutes at 0° C., then for 18 hours at room temperature. The solvent was evaporated. The residue (11 g) was purified over silica gel on a glass filter (eluent: DCM/CH₃OH 99/1). The desired fractions were collected and the solvent was evaporated. Toluene was added and co-evaporated on the rotary evaporator. Yield: 9.18 g of intermediate 56.

### b. Preparation of Intermediate 57

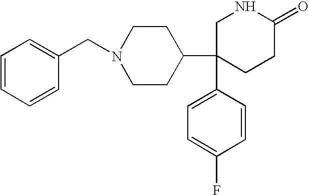

In a pressure vessel, a solution of intermediate 56 (0.023 mol) in CH₃OH (100 ml) was hydrogenated for 3 days at 50° C. (125 kg pressure) with Raney Nickel (q.s.) as a catalyst. After uptake of H₂ (2 equiv), the catalyst was filtered off and the filtrate was evaporated. Yield: 8.5 g of intermediate 57.

### c. Preparation of Intermediate 58 and Intermediate 16

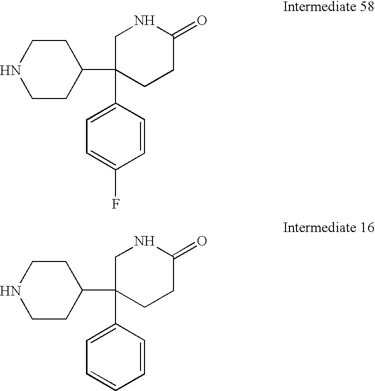

A solution of intermediate 57 (0.023 mol) in CH₃OH, p.a. (150 ml) was hydrogenated with Pd/C 10% (2 g) as a catalyst. After uptake of H₂ (1 equiv), the catalyst was filtered off and the filtrate was evaporated, then co-evaporated with methanol, yielding a crude fraction (mixture of desired fractions and side-product fractions, i.e. de-fluorated fractions). This fraction was separated and purified by reversed-phase high-performance liquid chromatography (NH₄HCO₃ in H₂O)/CH₃CN gradient). The desired product fractions were combined and the solvent was evaporated, then co-evaporated with methanol (2×). The residue was triturated under diethyl ether, then filtered off, washed with diethyl ether, and dried (vacuum, 50° C.). Yield: 1.7 g of intermediate 58 (26.7%). The side-product fractions were combined and the solvent was evaporated, then co-evaporated with methanol. The residue was triturated under diethyl ether, then filtered off, washed, and dried (vacuum, 50° C.). Yield: 0.83 g of intermediate 16 (14%).

### Example A5

### a) Preparation of Intermediate 17

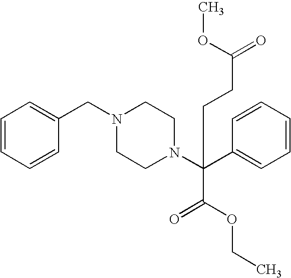

NaH (60% in paraffine) (0.0323 mol) was dissolved in DMF, p.a. dried on molecular sieves (30 ml) and the mixture was stirred at room temperature under N₂ atmosphere. A solution of α-phenyl-4-(phenylmethyl)-4-piperazineacetic acid ethyl ester (0.0269 mol) in DMF, p.a., dried on molecular sieves (70 ml) was added dropwise. The resultant mixture was stirred for 5 hours at 45-50° C., then cooled on an ice-bath. A solution of 3-bromopropanoic acid methyl ester (0.03 mol) in DMF, p.a. dried on molecular sieves (10 ml) was added dropwise. The reaction mixture was stirred vigorously for one hour at 0° C., then for 18 hours at room temperature. The solvent was evaporated. The residue was stirred in water and the product was extracted with DCM. The separated organic layer was dried (MgSO₄), filtered and the solvent evaporated. The residue was purified over silica gel on a glass filter (eluent: DCM/CH₃OH 98/2). The desired fractions were collected and the solvent was evaporated. Toluene was added and co-evaporated on the rotary evaporator. Yield: 7.8 g of intermediate 17.

### b) Preparation of Intermediate 18

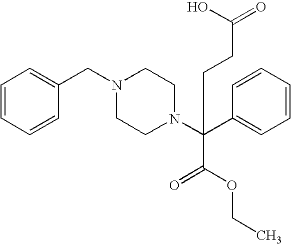

A solution of intermediate 17 (0.0162 mol) in 1,4-dioxane, p.a., (25 ml) was stirred. NaOH (1N) (25 ml) was added and the reaction mixture was stirred at room temperature for 65 hours. Then a HCl solution (25 ml, 1N) was added and the product was extracted with DCM. The separated organic layer was dried (MgSO₄), filtered and the solvent was evaporated, yielding intermediate 18 used as such in the next step.

### c) Preparation of Intermediate 19

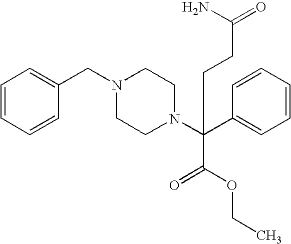

Reaction under N₂ atmosphere. Intermediate 18 (0.016 mol) was dissolved in DCM, p.a. (125 ml) and stirred at room temperature. 1,1′-Carbonylbis-1H-imidazole (3.25 g) was added. The reaction mixture was stirred for 90 minutes at room temperature. More 1,1′-carbonylbis-1H-imidazole (3 g) was added and the reaction mixture was stirred for 18 hours at room temperature. The reaction mixture was treated with NH₃ (excess of gas) over 35 minutes. Then stirring was continued for 18 hours at room temperature. The reaction mixture was washed with water, dried (MgSO₄), filtered and the solvent was evaporated. Toluene was added and co-evaporated on the rotary evaporator. Yield: 6.7 g of intermediate 19.

### d) Preparation of Intermediate 20

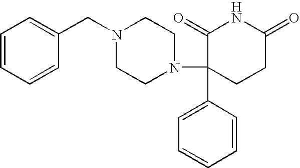

Reaction under N₂ atmosphere. Intermediate 19 (0.0163 mol) was dissolved in THF, p.a. dried over molecular sieves (80 ml) and stirred at room temperature. 2-Methyl-2-propanol sodium salt (1.9 g) was added. The reaction mixture was stirred for 18 hours at room temperature. More 2-methyl-2-propanol sodium salt (0.4 g) was added and the reaction mixture was stirred for 5 hours at room temperature. The mixture was poured out into water (200 ml). DCM (200 ml) was added. While stirring vigorously, acetic acid was added dropwise until a clear biphasic mixture resulted. The organic layer was separated, dried (MgSO₄), filtered and the solvent was evaporated. The residue was combined with analogously obtained fraction (0.37 g) and then purified over silica gel on a glass filter (eluent: DCM/CH₃OH 97/3). The desired fractions were collected and the solvent was evaporated. Toluene was added and co-evaporated on the rotary evaporator. Yield: 3.2 g of intermediate 20 (54%).

### e) Preparation of Intermediate 21

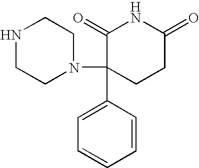

A solution of intermediate 20 (0.00853 mol) in CH₃OH, p.a. (150 ml) was hydrogenated for a few hours with Pd/C 10% (1 g) as a catalyst. After uptake of H₂ (1 equiv), the catalyst was filtered off and the filtrate was evaporated. The oily residue was triturated in diethyl ether/ethanol 9/1 (30 ml), then the precipitate was filtered off, washed and dried (vacuum, 55° C.). This fraction was stirred in water (10 ml) for 2 hours, then filtered off, washed with water, then DIPE, and then it was dried (vacuum, 60° C.). Yield: 0.44 g of intermediate 21 (18.9%).

### Example A6

### a) Preparation of Intermediate 22

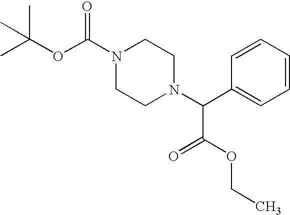

A solution of 1,1-dimethylethyl 1-piperazinecarboxylic acid ester (0.0279 mol) and Et₃N (0.03 mol) in DCM (80 ml) was stirred. α-bromo-benzeneacetic acid ethyl ester (0.028 mol) was added and the reaction mixture was stirred further at room temperature for 18 hours. The mixture was washed with H₂O and with a saturated aqueous NaHCO₃ solution. The organic layer was separated and dried (MgSO₄), filtered and the solvent was evaporated and co-evaporated with toluene, yielding intermediate 22 used as such in the next step.

### b) Preparation of Intermediate 23

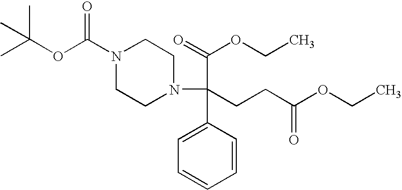

A mixture of NaH (0.0323 mol) in DMF (p.a.; dried on molecular sieves; 30 ml) was stirred under N₂ flow. A solution of intermediate 22 (0.027 mol) in DMF (p.a.; dried on molecular sieves; 70 ml) was added dropwise and the resulting mixture was stirred further at 45-50° C. for 5 hours. The reaction mixture was cooled to 0° C. on an ice-bath, and a solution of 3-bromo-propanoic acid ethyl ester (0.0297 mol) in DMF (p.a.; dried on molecular sieves; 10 ml) was added dropwise. The mixture was stirred further at 0° C. for 1 hour and then at room temperature for 18 hours. The solvent was evaporated and the residue was stirred in H₂O. This mixture was extracted with DCM. The separated organic layer was dried (MgSO₄), filtered, and the solvent was evaporated. The residue was purified over silica gel on a glass filter (eluent: DCM/CH₃OH 99/1). The product fractions were collected and the solvent was evaporated and co-evaporated with CH₃CH₂OH. Yield: 4.3 g of intermediate 23 (35.5%).

### c) Preparation of Intermediate 24

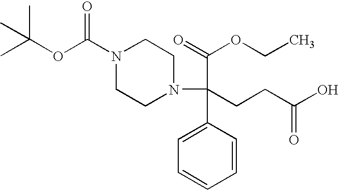

A solution of intermediate 23 (0.00959 mol) in 1,4-dioxane p.a. (15 ml) was stirred. NaOH (1N solution) (0.015 mol) was added and the reaction mixture was stirred further for 30 hours at room temperature. Then HCl (1N, 15 ml) was added and the product was extracted with DCM (2×). The separated organic layer was dried (MgSO₄), filtered, and the solvent was evaporated and co-evaporated with toluene, yielding intermediate 24 used as such in the next step.

### d) Preparation of Intermediate 25

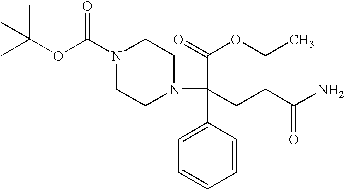

A solution of intermediate 24 (0.0095 mol) in DCM p.a. (80 ml) was stirred under N₂ flow. 1,1′-carbonylbis-1H-imidazole (0.015 mol) was added and the reaction mixture was stirred further for 18 hours at room temperature. An extra amount of 1,1′-carbonylbis-1H-imidazole (0.0123 mol) was added and the stirring of the mixture mixture was continued for 70 hours at room temperature. Then the mixture was treated with a flow of NH₃ flow (excess) for 45 minutes at room temperature. Precipitation occurred and the reaction mixture was stirred further for 1 hour at room temperature. H₂O (60 ml) was added and the resulting biphasic solution was stirred vigorously for 1 hour. The layers were separated. The water layer was extracted with DCM/CH₃OH 95/5 and the combined organic layers were dried (MgSO₄), filtered and evaporated. The remaining solid was stirred in Et₂O, filtered off and dried (vacuum, 55° C.). Yield: 3.5 g of intermediate 25 (87.8%)

### e) Preparation of Intermediate 26

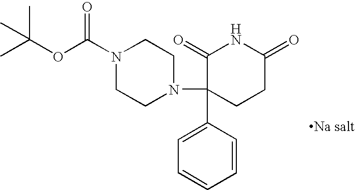

A mixture of intermediate 25 (0.0081 mol) in THF (p.a.; dried on molecular sieves; 60 ml) was stirred under N₂ flow. 2-methyl-2-propanol sodium salt (0.0105 mol) was added and the reaction mixture was stirred further for 3 days at room temperature. The solid part was filtered off and washed with THF, yielding intermediate 26. The precipitate was immediately used as such in the next reaction step A6.f).

### f) Preparation of Intermediate 27

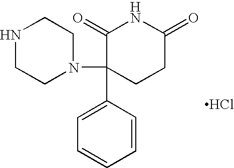

A mixture of intermediate 26 (0.008 mol) in 6N HCl/2-propanol (30 ml) was stirred at room temperature for 20 hours. The solid part was filtered off, washed with 2-propanol and dried (vacuum, 60° C.). Yield: 3.0 g of intermediate 27.

### Example A7

### a) Preparation of Intermediate 28

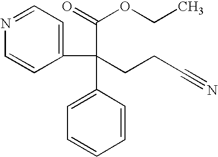

A solution of α-phenyl-4-pyridine acetic acid ethyl ester (0.182 mol) in 2-propenenitrile (12.6 ml) and 1,4-dioxane (p.a.; dried on molecular sieves; 300 ml) was stirred under N₂ atmosphere on an ice-bath. When stirring became difficult, Triton-B (4 ml) was added and the reaction mixture was stirred further with cooling for 30 minutes and at room temperature for 18 hours. The solvent was evaporated and the residue was purified over silica gel on a glass filter (eluent: DCM/CH₃OH 99/1). The product fractions were collected and the solvent was evaporated and co-evaporated with toluene. Yield: 44 g of intermediate 28.

### b) Preparation of Intermediate 29

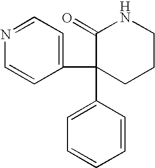

A mixture of intermediate 28 (0.136 mol) in THF, p.a. (400 ml) was hydrogenated with Raney Nickel (3 g) as a catalyst. After uptake of H₂ (2 equiv), the catalyst was filtered off and the filtrate was evaporated (water-bath temperature: 70° C.). The residue stood over the weekend. The liquid layer was decanted off. The solid residue was stirred in diethyl ether, filtered off, washed with diethyl ether, then dried (vacuum, 55° C.). Yield: 11.4 g of intermediate 29.

### c) Preparation of Intermediate 30

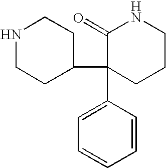

A solution of intermediate 29 (0.0448 mol) in CH₃OH (150 ml) was hydrogenated at 75° C. under 90 atm of pressure with Pd/C 10% (1 g) as a catalyst. After uptake of H₂ (3 equiv), the catalyst was filtered off and the filtrate was evaporated. The oily residue was triturated under diethyl ether, filtered off, washed, then dried (vacuum, 55° C.). Yield: 9.3 g of intermediate 30 (80.4%).

### Example A8

### a) Preparation of Intermediate 31

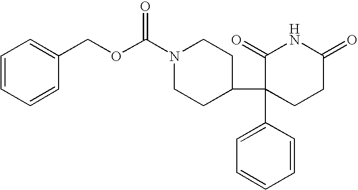

Phenylmethyl carbonochloridic acid ester (0.116 mol) was added dropwise to a stirring mixture of 3-phenyl-[3,4′-bipiperidine]-2,6-dione (0.1 mol), DMF, p.a. (300 ml) and Et₃N (0.3 mol). The reaction mixture was stirred for 18 hours at room temperature. The mixture was poured out into cold water (1 L). The product was extracted with diethyl ether (2×1 L). The combined organic layers were dried (MgSO₄), filtered and the solvent was evaporated. The residue was stirred in diethyl ether (200 ml), filtered off, washed, and then dried (vacuum, 50° C.). Yield: 29 g of intermediate 31.

### b) Preparation of Intermediate 32

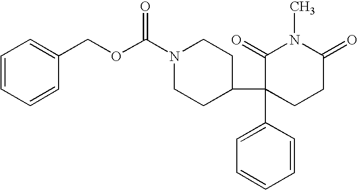

A solution of intermediate 31 (0.0123 mol) in DMF, p.a. (100 ml) was stirred. K₂CO₃ (0.0135 mol) and CH₃I (0.0135 mol) were added and the reaction mixture was stirred further for 18 hours at room temperature. Then the reaction mixture was poured slowly into cold water (600 ml) and stirred for 15 minutes. The precipitate was filtered off, washed with H₂O and dried (vacuum, 55° C.). The product was taken up in DCM, dried (MgSO₄), filtered and the solvent was evaporated and co-evaporated with toluene. Yield: 6.0 g of intermediate 32.

### c) Preparation of Intermediate 33

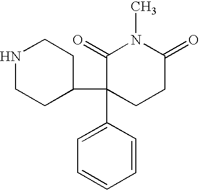

A solution of intermediate 32 (0.014 mol) in CH₃OH, p.a. (150 ml) was hydrogenated with Pd/C 10% (2 g) as a catalyst. After uptake of H₂ (1 equiv), the catalyst was filtered off and the filtrate was evaporated, yielding intermediate 33 used as such in the next step.

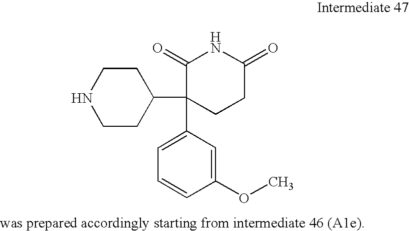

### Example A9

### a) Preparation of Intermediate 34 and Intermediate 34a

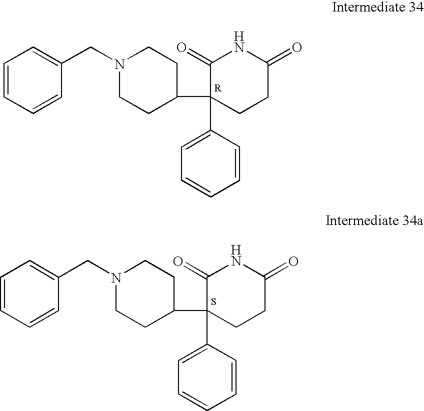

NaHCO₃ (0.5 mol) was dissolved in H₂O (350 ml). This solution was added to a mixture of benzetimide hydrochloride (0.209 mol) in DCM (400 ml). The reaction mixture was stirred for 18 hours at room temperature. The organic layer was separated, dried (MgSO₄), filtered and the solvent was evaporated. Ethanol was added and co-evaporated. The residue was separated into its enantiomers by chiral column chromatography over an OJ column (eluent: ethanol/heptane 60/40). Two product fraction groups were collected and their solvent was partially evaporated (to a ±150 ml concentrate). Crystallization occurred in each concentrate. Each precipitate was filtered off, washed with ethanol, and dried (vacuum, 50° C.). Yield: 21.2 g of intermediate 34a (S configuration) and 27.0 g of intermediate 34 (R configuration).

### b) Preparation of Intermediate 35

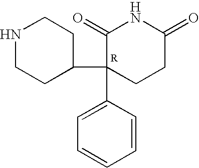

A solution of intermediate 34 (0.074 mol) in CH₃OH, p.a. (250 ml) was hydrogenated with Pd/C 10% (3 g) as a catalyst. After uptake of H₂ (1 equiv), the catalyst was filtered off and the filtrate was evaporated. The residue was stirred in diethyl ether, then filtered off, washed with diethyl ether, then dried (vacuum, 50° C.). Yield: 18.8 g of intermediate 35 (93.3%).

### Example A10

### a) Preparation of Intermediate 36

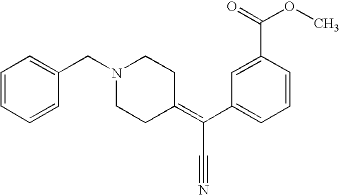

Reaction under N₂ atmosphere. A solution of 3-cyanomethylbenzoic acid methyl ester (0.0314 mol) in CH₃OH, p.a. (60 ml) was stirred at room temperature. CH₃ONa 30% (0.032 mol) was added. 1-(Phenylmethyl)-4-piperidinone (0.0157 mol) was added and the reaction mixture was stirred and refluxed for 18 hours. The mixture was allowed to cool to room temperature. The mixture was poured out into ice-water (180 ml). The product was extracted with DCM (2×). The combined organic layers were dried (MgSO₄), filtered and the solvent was evaporated. The residue (7 g) was purified over silica gel on a glass filter (eluent: DCM/CH₃OH 99.5/0.5). The product fractions were collected and the solvent was evaporated. EtOAc was added and co-evaporated. Yield: 1.38 g of intermediate 36.

### b) Preparation of Intermediate 37

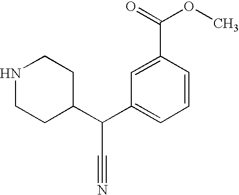

A solution of intermediate 36 (0.004 mol) in CH₃OH, p.a. (50 ml) was hydrogenated with Pd/C 10% (0.5 g) as a catalyst. After uptake of H₂ (2 equiv), the catalyst was filtered off and the filtrate was evaporated. Yield: 0.87 g of intermediate 37 (84.2%).

### c) Preparation of Intermediate 38 and 38a

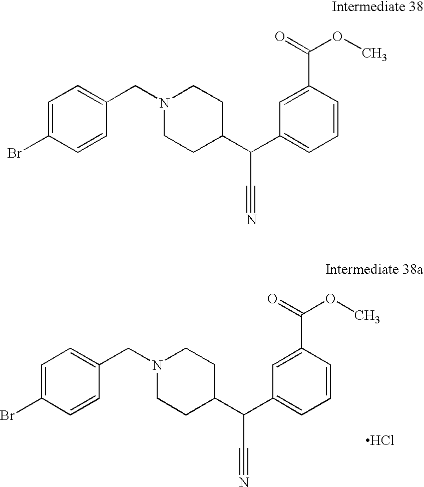

A mixture of intermediate 37 (0.00337 mol) and N-ethyl-N-(1-methylethyl)-2-propanamine (0.00337 mol) in DCM, p.a. (20 ml) and DMF, p.a. (10 ml) was stirred at room temperature. 1-Bromo-4-(chloromethyl)benzene (0.00337 mol) was added and the reaction mixture was stirred for 65 hours at room temperature. The reaction mixture was washed with water, dried (MgSO₄), filtered and the solvent was evaporated. The residue was dissolved in 2-propanol (20 ml) and converted into the hydrochloric acid salt (1:1) with 6 N HCl/2-propanol (1.5 ml). The solvent was evaporated, yielding intermediate 38a. The residue (oil) was stirred in diethyl ether. The solvent was evaporated. The residue was re-converted into the free base, then purified by Flash chromatography (eluent: DCM/CH₃OH 99.7/0.3). The product fractions were collected and the solvent was evaporated. 1,4-Dioxane was added and co-evaporated. Yield: 0.92 g of intermediate 38 (63.9%).

### d) Preparation of Intermediate 39

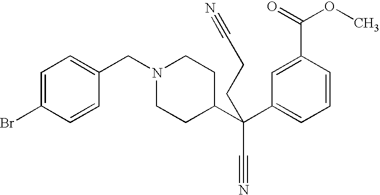

Reaction under N₂ atmosphere. A solution of intermediate 38 (0.00213 mol) in 2-propenenitrile (0.17 ml, 0.00256 mol) and 1,4-dioxane (p.a.; dried on molecular sieves; 15 ml) was stirred and cooled on an ice-bath. When stirring became difficult, Triton-B (0.05 ml) was added and the reaction mixture was stirred for 15 minutes while cooling on the melting ice-bath. Then, the reaction mixture was stirred overnight at room temperature. The solvent was evaporated, yielding intermediate 39 used as such in the next step B6.

### Example A11

### a-1) Preparation of Intermediate 40

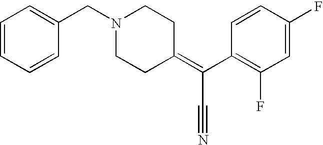

CH₃ONa (0.0170 mol) was added to a solution of 1-(phenylmethyl)-4-piperidinone (0.0163 mol) and 2,4-difluorobenzeneacetonitrile (0.0327 mol) in CH₃OH, dry (50 ml) under argon, and the mixture was stirred under reflux for 4 hours. Then, the reaction mixture was cooled to room temperature and poured into ice (200 g). The resulting mixture was extracted with ethyl acetate. The separated organic layer was dried (Na₂SO₄), filtered and the solvent was evaporated in vacuum. Yield: 5.3 g of intermediate 40.

### a-2) Preparation of Intermediate 40

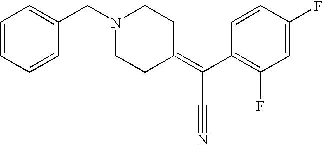

CH₃ONa (47.2 ml, 0.26 mol; 30% in CH₃OH) was added to a stirring solution of 2,4-difluorobenzeneacetonitrile (39.7 g, 0.259 mol) and 1-(phenylmethyl)-4-piperidinone (24.5 g, 0.129 mol) in CH₃OH (250 ml; p.a.) under N₂ atmosphere. The reaction mixture was stirred and refluxed for 18 hours. The solvent was evaporated and the residue was stirred in 250 ml ice-H₂O. The product was extracted 2× with DCM. The combined organic layers were dried (MgSO₄), filtered and the solvent was evaporated. The residue was filtered over silica (eluent: DCM/MeOH 99.5/0.5). The pure fractions were combined and the solvent was evaporated and co-evaporated with toluene. Yield: 27.6 g of intermediate 40.

### b-1) Preparation of Intermediate 41

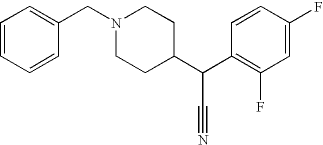

NaBH₄ (0.0245 mol) was added to a solution of intermediate 40 (0.0163 mol) in 2-propanol (20 ml). The mixture was stirred under reflux for 4 hours, and cooled to room temperature. Then, a mixture of water and ice (200 ml) was added, and extracted with dichloromethane. Extract was dried over Na₂SO₄, filtered and concentrated in vacuum. The residue was purified by Flash chromatography (eluent: hexane/ethyl acetate 4/1). The product fractions were collected and the solvent was evaporated. Yield: 3.464 g of intermediate 41 (65%; 2-(1-benzyl-4-piperidinyl)-2-(2,4-difluorophenyl)acetonitrile).

### b-2) Preparation Of Intermediate 41

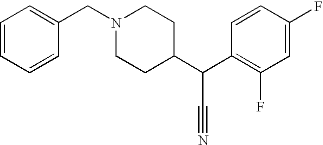

A solution of intermediate 40 (27 g, 0.083 mol) in a thiophene solution (2 ml) and CH₃OH (250 ml; p.a.) was hydrogenated over Pd/C 10% (3 g, catalyst). After the calculated amount of H₂ (1 equivalent) was taken up, the catalyst was filtered off. The filtrate was evaporated and co-evaporated with 1,4-dioxane. The residue was used as such in the next step. Yield: Intermediate 41 (residue).

### c-1) Preparation of Intermediate 42

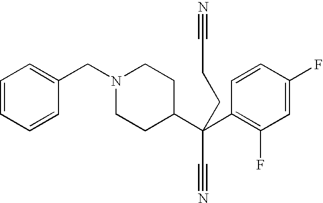

2-propenenitrile (0.0149 mol) was added to a solution of intermediate 41 (0.0106 mol) in dioxane (25 ml), stirred at 0° C. under argon atmosphere. After freezing of dioxane (in 20 minutes) Triton B (0.0149 mol) was added. The mixture was stirred at room temperature for about 4 hours. Then, the mixture stood overnight under argon flow. The solvent was evaporated. Yield: 3.652 g of intermediate 42.

### c-2) Preparation of Intermediate 59

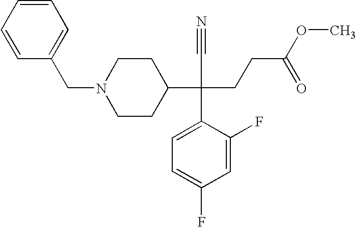

A solution of intermediate 41 (0.083 mol; residue) and methyl acrylate (9 ml, 0.1 mol) in 1,4-dioxane (250 ml; p.a., dried on molecular sieves) was stirred under N₂ atmosphere on an ice-bath. When stirring became difficult, Triton-B (1 ml; catalyst) was added and the reaction mixture was continued stirring on the ice-bath for 5 minutes and then at room temperature for 3 days. Then more methyl acrylate (3 ml) and Triton-B (0.5 ml) were added and the reaction mixture was continued stirring for 18 hours at room temperature. The solvents were evaporated. The residue was filtered over silica (eluent: DCM/CH₃OH 99/1). The desired fractions were combined and evaporated and co-evaporated with toluene. Yield: 32 g of intermediate 59 (93.5%).

### d-1) Preparation of Intermediate 43

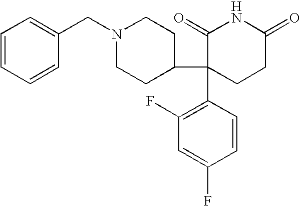

H₂SO₄ (8.7 ml) was added to a mixture of intermediate 42 (1 0.010 mol) in acetic acid (36 ml) under stirring and cooling. The reaction mixture was stirred for 4 hours at reflux. Then, the mixture was poured into ice, and a 50% solution of NaOH (30 ml) was added. The resulting mixture was extracted with DCM. The layers were separated. The aqueous layer was neutralized with K₂CO₃, and extracted with DCM again. The organic extracts were combined, dried over Na₂SO₄, filtered and the filtrate's solvent was evaporated in vacuum. The residue was subjected to Flash tube (eluent: DCM). The product fractions were collected and the solvent was evaporated. Yield: 1.454 g of intermediate 43 (34%).

### d-2) Preparation of Intermediate 43

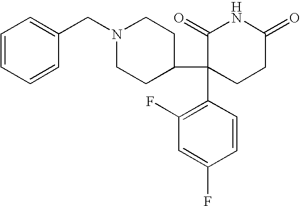

H₂SO₄ (30 ml) was added to a stirring solution of intermediate 59 (20 g, 0.0485 mol) in CH₃COOH and then stirred in an oil bath at 170° C. for 3 hours. The reaction mixture was allowed to reach 55° C. and was then poured slowly into a stirring mixture of ice (400 g) and NaOH (50%, 115 ml). NaHCO₃ was added portionwise to the mixture until pH=8 to 9. This mixture was then extracted with DCM. The separated organic layer was dried (MgSO₄), filtered and the filtrate's solvent was evaporated. The residue was filtered over silica (eluent: DCM/CH₃OH 97/3). The desired product fractions were collected and the solvent was evaporated, and co-evaporated with EtOH. Yield: 16.8 g of intermediate 43 (86.9%).

### e) Preparation of Intermediate 44

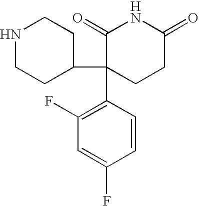

A solution of intermediate 43 (0.00427 mol) in CH₃OH, p.a. (150 ml) was hydrogenated with Pd/C 10% (0.5 g) as a catalyst. After uptake of H₂ (1 equiv), the catalyst was filtered off and the filtrate was evaporated and co-evaporated with CH₃OH, yielding intermediate 44 used as such in the next step (B19).

### f) Preparation of Intermediate 60 and Intermediate 61

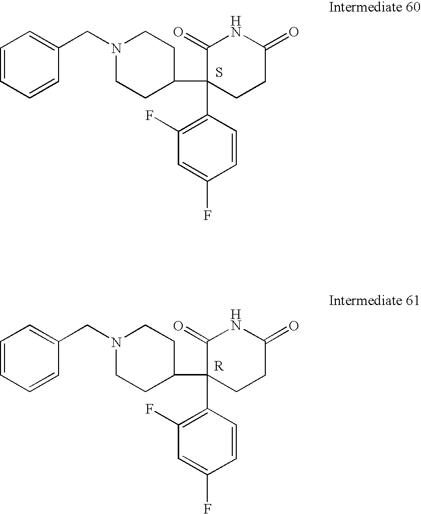

Intermediate 43 was chirally separated by chiral column chromatography (AD-chiral PACK Diacel column, eluent: EtOH/hexane 50/50). The 2 desired product fractions were collected and both were concentrated to an end volume of 100 ml. Both precipitates were filtered off and washed with DIPE. Finally, both precipitates were dried (50° C., vacuum). Yield: 3.3 g of intermediate 60 (S configuration, Optical Rotation=−) Yield: 4.3 g of intermediate 61 (R configuration, Optical Rotation=+).

### g) Preparation of Intermediate 62

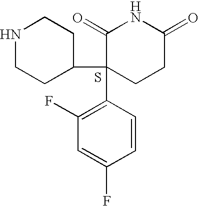

A solution of intermediate 60 (3.2 g, 0.008 mol) in CH₃OH (150 ml; p.a.) was hydrogenated at 50° C. with Pd/C 10% (1 g, catalyst). After an uptake of H₂ (1 eq), the catalyst was filtered off. The filtrate's solvent was evaporated and co-evaporated with 2-propanol. The residue was stirred in CH₃OH, the precipitate was filtered off, washed and dried (vacuo, 50° C.). Yield: 0.69 g of intermediate 62. The filtrate's solvent was evaporated. The residue was stirred in EtOH, the precipitate was filtered off, washed with EtOH and dried (50° C., vacuum). Yield: 0.84 g of intermediate 62 (total yield: 49.4%).

### h) Preparation of Intermediate 63

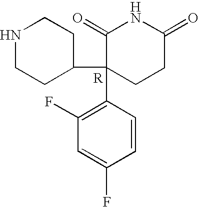

A solution of intermediate 61 (4.2 g, 0.0105 mol) in CH₃OH (150 ml) was hydrogenated at room temperature with Pd/C 10% (1 g, catalyst). After an uptake of H₂ (1 eq), the catalyst was filtered off. The filtrate's solvent was evaporated. The residue was re-crystallized in EtOH. The precipitate was filtered off, washed with EtOH and dried (vacuum, 50° C.). Yield: 1.68 g of intermediate 63 (51.9%).

### Example A12

### Preparation of Intermediate 53

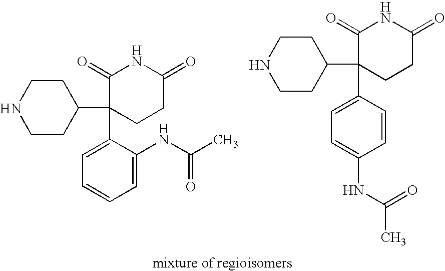

A mixture of compound 188 (prepared according to B17c)(0.002 mol) in THF (100 ml) was hydrogenated overnight at room temperature with Pd/C, 10% (0.3 g) as a catalyst and then again overnight at 50° C. The mixture was filtered and a fresh amount of Pd/C 10% (0.3 g) was added. The mixture was hydrogenated overnight at 50° C. Then an extra fresh amount of Pd/C 10% (0.5 g) was added and the mixture was hydrogenated overnight at 50° C. After uptake of H₂ (1 equiv), the catalyst was filtered off and the filtrate was evaporated. Yield: 1.140 g of intermediate 53.

### Example A13

### Preparation of Intermediate 54

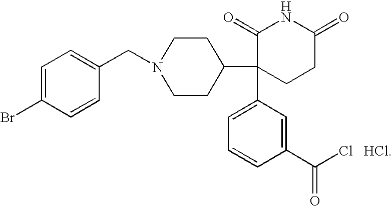

A mixture of compound 23 (prepared according to B6) (0.0001 mol) in SOCl₂ (1 ml) was stirred at room temperature for 4 hours. DCM (1 ml) was added and the reaction mixture was stirred further at room temperature for 4 days. The solvent was evaporated, yielding intermediate 54 used as such in the next step (B9a).

Intermediate

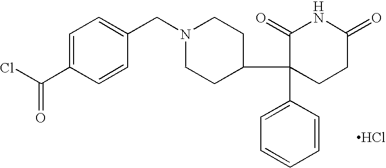

was prepared accordingly and is used to prepare compound 36 described in example B14.

### Example A14

### Preparation of Intermediate 55

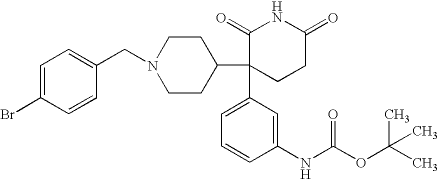

A mixture of compound 23 (prepared according to B6) (0.000107 mol), phosphorazidic diphenyl ester (0.00011 mol) and Et₃N (0.016 ml) in 2-methyl-2-propanol (2 ml) was stirred in a sealed tube at 85° C. for 18 hours. Then an extra amount of phosphorazidic diphenyl ester (0.00011 mol) was added and the reaction mixture was stirred further at 85° C. for 20 hours. The solvent was evaporated, yielding intermediate 55 used as such in the next step (B10).

### Example A15

### a) Preparation of Intermediate 64

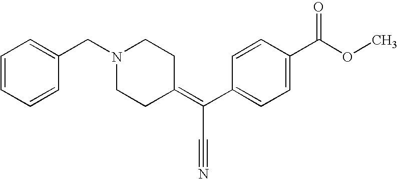

Reaction under N₂ flow. A solution of 4-cyanomethylbenzoic acid methyl ester (35.0 g, 0.2 mol), 1-(phenylmethyl)-4-piperidone (18.9 g, 0.1 mol) and a 30% NaOCH₃ solution in CH₃OH (37.3 ml) in CH₃OH (300 ml; p.a.) was stirred and refluxed for 18 hours. Then the reaction mixture was stirred at room temperature for 24 hours. The reaction mixture was poured out into ice water (750 ml). This mixture was extracted 2 times with DCM. The separated organic layer was dried (MgSO₄), filtered and the solvent was evaporated. The residue was purified over silica (eluens: DCM/CH₃OH 99.5/0.5). The product fractions were collected. The solvent was evaporated and co-evaporated with toluene. Yield: 12.1 g of intermediate 64 (34.9%).

### b) Preparation of Intermediate 65

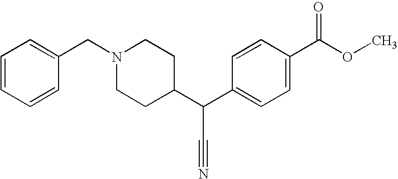

A mixture of intermediate 64 (12 g, 0.034 mol) in CH₃OH (250 ml) and THF (50 ml) was hydrogenated at 50° C. with Pd/C 10% (2 g) as the catalyst in the presence of a thiophene solution in CH₃OH (1 ml). After uptake of H₂ (1 eq) the catalyst was filtered off. The filtrate's solvent was evaporated and co-evaporated with 1,4-dioxane. Yield: intermediate 65 (used as such in next reaction step)

### c) Preparation of Intermediate 66

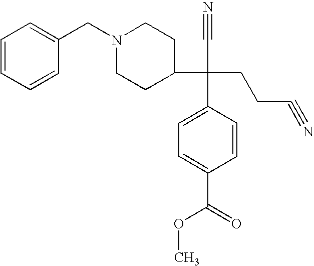

Reaction under N₂ flow. A solution of intermediate 65 (crude; approx. 0.033 mol) and 2-propenenitrile (2.63 ml, 0.04 mol) in 1,4-dioxane (125 ml, dried on molecular sieves) was stirred on an ice-bath. Triton B (0.4 ml) was added to the reaction mixture and stirred on the ice-bath for 1 minute. Then the reaction mixture was stirred at room temperature for 18 hours. The solvent was evaporated. Yield: intermediate 66 (used as such in next reaction step).

### d) Preparation of Intermediate 67

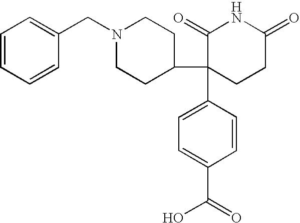

H₂SO₄ (15 ml) was added to a solution of intermediate 66 (crude; approx. 0.032 mol) in CH₃COOH and then heated to 165° C. on an oil-bath. The reaction mixture was stirred and refluxed for 3.5 hours. The reaction mixture was allowed to reach 50° C. and was then poured out into ice water (750 ml). K₂CO₃ was added portionwise to the reaction mixture until the pH=5. This mixture was extracted with DCM/CH₃OH (90/10) to result in a separated aqueous layer and a separated organic layer. K₂CO₃ was added portionwise to the separated aqueous layer until pH was 7. Then DCM/CH₃OH (3050 ml; 90/10) was added to the mixture and stirred for 1 hour. The precipitate was filtered off and washed with H₂O. This precipitate was stirred in boiling CH₃CN (60 ml) for 45 minutes. The precipitate was filtered off hot, washed and dried (50° C., vacuum). The residue was stirred in boiling H₂O (200 ml) for 1 hour. The precipitate was filtered off hot, washed and dried (55° C., vacuum with airstream). Yield: 6.1 g of intermediate 67 (46.9%).

### e) Preparation of Intermediate 68

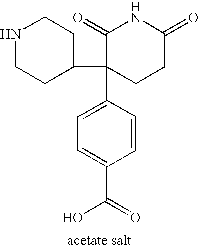

A solution of intermediate 67 (6.1 g, 0.015 mol) in CH₃COOH (150 ml) was hydrogenated with Pd/C 10% (1 g) as the catalyst. After uptake of H₂ (1 eq), the catalyst was filtered off. The filtrate's solvent was evaporated and co-evaporated with toluene. The residue was stirred in Et₂O. The precipitate was filtered off, washed with Et₂O and dried (55° C., vacuum). Yield: 4.8 g of intermediate 68 (85%).

### Example A16

### a) Preparation of Intermediate 69

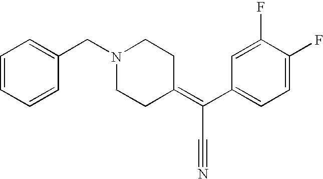

Na (1.158 g, 0.0503 mol) was dissolved in CH₃OH (20 ml; dry). This solution was added to a mixture of 3,4-difluorobenzeneacetonitrile (7.578 g, 0.0495 mol) in CH₃OH (80 ml; dry) at room temperature under argon atmosphere. Then 1-(phenylmethyl)-4-piperidinone (5.882 ml, 0.033 mol) was added and the mixture was stirred and refluxed for 8 hours. Then, the reaction mixture was cooled to room temperature and poured into cold H₂O. The resulting mixture was extracted with DCM (2×70 ml), dried (Na₂SO₄), filtered and the solvent was evaporated. The residue (12.9 g, brown red oil) was purified by flash chromatography (Merck silica gel 60, 230-400 mesh, eluent: DCM). The desired fractions were collected and the solvent was evaporated. Yield: 11.4 g of intermediate 69 (brown-yellow oil; used as such in the next step).

### b) Preparation of Intermediate 70

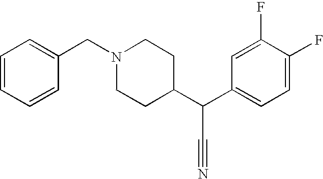

NaBH₄ (1.994 g, 0.0527 mol) was added to a solution of intermediate 69 (11.4 g, 0.0351 mol) in 2-propanol (200 ml). The mixture was stirred and refluxed for 2 hours and was then cooled to room temperature. A mixture of water and ice (300 ml) was added and the resulting mixture was extracted with DCM (2×300 ml). The separated organic layer was dried (Na₂SO₄), filtered and the solvent was evaporated. The residue (10.465 g as a brown-red oil) was purified by flash chromatography (Merck silica gel 60, 230-400 mesh; eluent: DCM). The desired fractions were collected and the solvent was evaporated. Yield: 7.58 g of intermediate 70 (70% yield based on starting material of A16a).

### c) Preparation of Intermediate 71

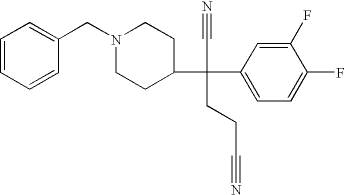

3-propenenitrile (2.156 ml, 0.03251 mol) was added to a solution of intermediate 70 (7.58 g, 0.02322 mol) in dioxane (80 ml; dry) at 0° C. under argon. After 10 minutes, Triton B (0.392 ml) was added at 0° C. under argon. The reaction mixture was stirred at room temperature for 20 hours. The solvent was evaporated and the residue was dissolved in DCM and filtered through a layer of silica gel. The solvent was evaporated. Yield: 8.81 g of intermediate 71 (yellow oil; used a such in the next step without additional purification).

### d) Preparation of Intermediate 72

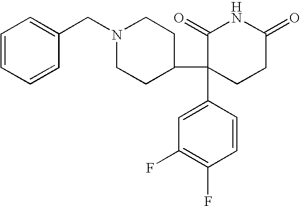

H₂SO₄ (15.8 ml; 96%) was added dropwise to a mixture of intermediate 71 (9.55 g, 0.02013 mol; 80% pure) and CH₃COOH (52 ml) under stirring and cooling (exothermic). The reaction mixture was stirred for 4 hours at reflux temperature. Then, the mixture was poured into ice and an aqueous NaOH solution (30%) was added to adjust the pH=7. The resulting mixture was extracted with a mixture of DCM and CH₃OH (10:1). The extract was washed with an aqueous K₂CO₃ solution (10%). The original aqueous layer was neutralized with an aqueous K₂CO₃ solution (10%) until the pH=8-9, and was then extracted again with the mixture of DCM/CH₃OH (10:1). The organic layers were combined, dried (Na₂SO₄), filtered and the solvent was evaporated. The residue was triturated with ether. The precipitate was filtered off and washed with ether. Yield: 4.825 g of intermediate 72 (52% yield based on the starting material of A 16c).

### e) Preparation of Intermediate 73

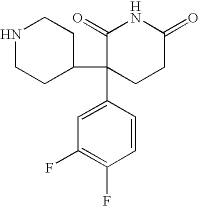

A mixture of intermediate 72 (4.7 g, 0.0118 mol) in CH₃OH (150 ml; p.a.) was hydrogenated with Pd/C 10% (0.5 g) as the catalyst. After uptake of H₂ (1 eq), the catalyst was filtered off. CH₃OH was added to the filtrate and stirred. The precipitate was filtered off and then dried (50° C., vacuum). Yield: 2.1 g of intermediate 73 (57.7%).

### Example A 17

### a) Preparation of Intermediate 74

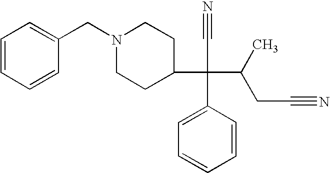

α-Phenyl-1-(phenylmethyl)-4-piperidineacetonitrile (2.00 g, 0.0069 mol; CAS [7254-21-9]) and 2-butenenitrile (0.647 g, 0.00966 mol) were mixed up in dioxane (30 ml; dry) under argon atmosphere while cooling on an ice-bath. After 20 minutes, Triton B (0.116 ml) was added, and the ice-bath was removed. The reaction mixture was stirred for 2 days at room temperature. Then an extra amount of 2-butenenitrile (0.647 g, 0.00966 mol) and Triton B (0.116 ml) were added to the reaction mixture, and it was heated for 24 hours at 50-60° C. Then potassium tert-butylate (0.007 mol) was added to the reaction mixture, and the mixture was stirred at room temperature for 24 hours. Then the reaction mixture was concentrated, and the residue, was used in the next step without purification.

### b) Preparation of Intermediate 75

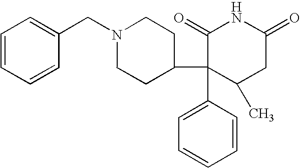

H₂SO₄ (5.7 ml, 96%) was added to a mixture of intermediate 74 (2.394 g, concentrated reaction mixture from previous step) and CH₃COOH (23.5 ml) while the mixture was stirred and cooled. The reaction mixture was stirred for 4 hours at 165° C. Then the mixture was treated with ice, and a 50% solution of NaOH (30 ml) was added. The resulting solution was extracted with DCM. The separated organic layer was dried (Na₂SO₄) and the solvent was evaporated. The residue was purified by column chromatography (eluent:dichloromethane). The desired fractions were collected and the solvent was evaporated. Yield: 0.15 g of intermediate 75.

### c) Preparation of Intermediate 76

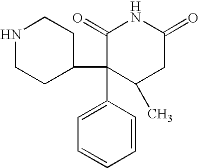

A solution of intermediate 75 (0.00133 mol, 0.5 g) in CH₃OH (50 ml; p.a.) was hydrogenated with Pd/C 10% (0.3 g) as the catalyst. After the calculated amount of H₂ (1 eq) was taken up, the catalyst was filtered off. The filtrate was evaporated. The residue was triturated in Et₂O and filtered off. It was then washed with Et₂O and dried at 50° C. (vacuum). Yield: 0.05 g of intermediate 76 (13.1%).

### B. Preparation of the Compounds

### Example B1

### a) Preparation of Compound 1

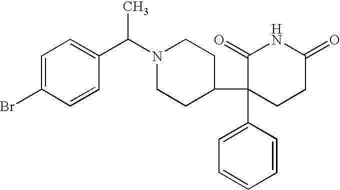

1-Bromo-4-(1-chloroethyl)benzene (0.00035 mol; 1 M in DCM) was added to a shaking mixture of 3-phenyl-[3,4′-bipiperidine]-2,6-dione (0.000275 mol) in Et₃N (0.2 ml) and DMF (p.a.; dried on molecular sieves; 5 ml) and then the reaction mixture was shaken for 18 hours at room temperature and for 5 hours at 55° C. The solvent was evaporated and the residue obtained was purified by reversed-phase high-performance liquid chromatography (method A). The product fractions were collected and the solvent was evaporated and co-evaporated with CH₃OH. Yield: 0.034 g of compound 1.

### b) Preparation of Compound 2

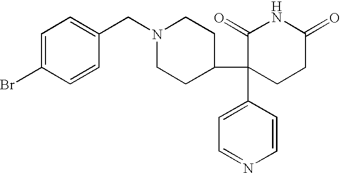

1-Bromo-4-(chloromethyl)benzene (0.0002 mol) was added to a shaking solution of

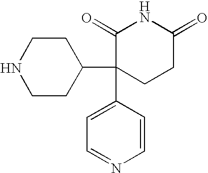

(prepared according to A1.f) (0.000183 mol) in Et₃N (0.2 ml) and DMF (p.a.; dried on molecular sieves; 6 ml). The reaction mixture was shaken further for 18 hours at room temperature, then for 50 minutes at 55° C. The solvent was evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were combined and the solvent was evaporated. Methanol was added and co-evaporated on the rotary evaporator. Yield: 0.007 g of compound 2.

### c) Preparation of Compound 3

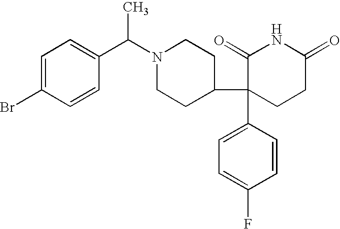

1-Bromo-4-(1-chloroethyl)benzene (0.0002 mol) was added to a shaking solution of 2-(p-fluorophenyl)-2-(4-piperidinyl)glutarimide (0.00014 mol) in Et₃N (0.2 ml) and DMF, p.a., dry (5 ml). The reaction mixture was shaken further for 18 hours at room temperature, then for 15 minutes at 55° C. The solvent was evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were combined and the solvent was evaporated. Methanol was added and co-evaporated on the rotary evaporator. Yield: 0.007 g of compound 3.

### d) Preparation of Compound 4

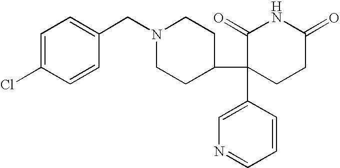

1-Chloro-4-(chloromethyl)benzene (0.00028 mol) was added to a shaking solution of intermediate 6 (prepared according to A1.f) (0.00019 mol) in Et₃N (0.2 ml) and DMF (5 ml). The reaction mixture was shaken further for 18 hours at room temperature, then for 20 minutes at 55° C. The solvent was evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were combined and the solvent was evaporated. Methanol was added and co-evaporated on the rotary evaporator. The obtained residue was further purified by Flash Tube (eluent: DCM/CH₃OH 84/16). The product fraction was isolated and stirred in DCM/methanol 90/10, then filtered to remove the silica gel and the filtrate was evaporated. Yield: 0.014 g of compound 4. Compounds 177, 178 and 180 were prepared accordingly. For compounds 179 and 184, this additional purification step was not performed.

### e) Preparation of Compound 5

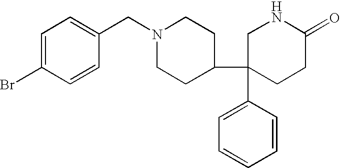

1-Bromo-4-(chloromethyl)benzene (0.00035 mol) was added to a shaking solution of intermediate 16 (prepared according to A4.b) (0.00019 mol) in Et₃N (0.2 ml) and DMF, p.a. (4 ml). The reaction mixture was shaken further for 18 hours at room temperature, then for 40 minutes at 55° C. The solvent was evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were combined and the solvent was evaporated. Methanol was added and co-evaporated on the rotary evaporator. Yield: 0.025 g of compound 5.

### f) Preparation of Compound 6

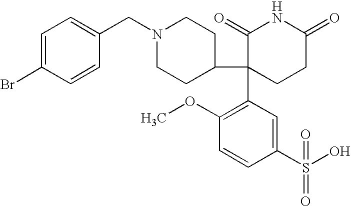

1-Bromo-4-(chloromethyl)benzene (0.00129 mol) was added to a stirring mixture of intermediate 9 (prepared according to A2.c) (0.00117 mol) and Et₃N (0.56 ml) in DMF, p.a. (10 ml). The reaction mixture was stirred for 65 hours at room temperature. H₂O (20 ml) was added to ameliorate dissolution of starting material intermediate 9). The reaction mixture was stirred for 20 hours at room temperature. H₂O (20 ml) was added and the mixture was washed with diethyl ether. The aqueous phase was saturated with NaCl. The product was extracted with DCM/methanol 90/10 (2×). The combined organic layers were dried (MgSO₄), filtered and the solvent was evaporated. Toluene was added and co-evaporated on the rotary evaporator. The residue (0.37 g) was purified by high-performance liquid chromatography over RP-18 (eluent: (0.5% NH₄OAc in H₂O)/CH₃CN 90/10). The product fractions were collected and the solvent was evaporated. The residue was stirred in DCM/water (2 ml/2 ml) for 18 hours. The precipitate was filtered off, washed with DCM and with water, again with DCM, and dried (vacuum, 50° C.). Yield: 0.13 g of compound 6.

### g) Preparation of Compound 7

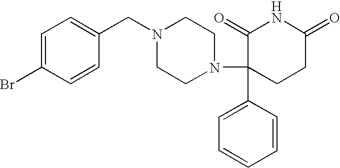

1-Bromo-4-(chloromethyl)benzene (0.00023 mol) was added to a stirring mixture of intermediate 21 (prepared according to A5.e) (0.00019 mol) and Et₃N (0.2 ml) in DMF (6 ml). The reaction mixture was stirred for 18 hours at room temperature. The solvent was evaporated. The residue was purified by reversed-phase high-performance liquid chromatography (Column: Xterra Prep MS C18, Length: 10 cm, I.D.: 19 mm, particle size: 5 μm; eluent: (0.5% NH₄OAc in H₂O)/CH₃OH/CH₃CN gradient). The desired fractions were collected and the solvent was evaporated. Yield: 0.044 g. For liberation of the free base, the fraction was stirred vigorously for 2 hours in a biphasic solution of DCM (4 ml) and a saturated aqueous NaHCO₃ solution (1 ml). The mixture was filtered through an Isolute HM-N filter and the filtrate was evaporated. Yield: 0.040 g of compound 7.

### h) Preparation of Compound 8

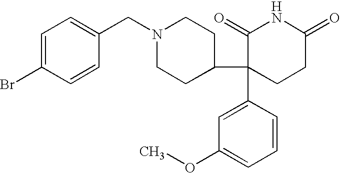

A mixture of intermediate 47 (prepared according to A8c) (0.00033 mol), 1-bromo-4-(chloromethyl)benzene (0.00049 mol) and Et₃N (2 ml) in DMF (15 ml) was stirred for 20 hours at room temperature. The solvent was evaporated. The residue was purified by HPLC (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were collected and the solvent was evaporated. Yield: 0.0469 g of compound 8.

### i) Preparation of Compound 9

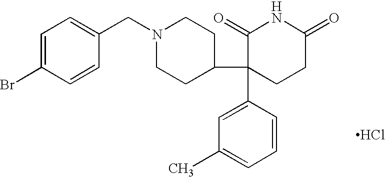

A mixture of 2-(4-piperidinyl)-2-m-tolylglutarimide (0.0014 mol), 1-bromo-4-(chloromethyl)benzene (0.0019 mol) and Et₃N (5 ml) in DMF (20 ml) was stirred for 7 hours at room temperature. The solvent was evaporated. The residue was purified by column chromatography over silica gel (eluent: DCM/CH₃OH 95/5). The product fractions were collected and the solvent was evaporated. The residue was converted into the hydrochloric acid salt (1:1) with HCl/2-propanol, filtered off and dried. Yield: 0.180 g of compound 9 (26.1%).

### j) Preparation of Compound 10 and Compound 87

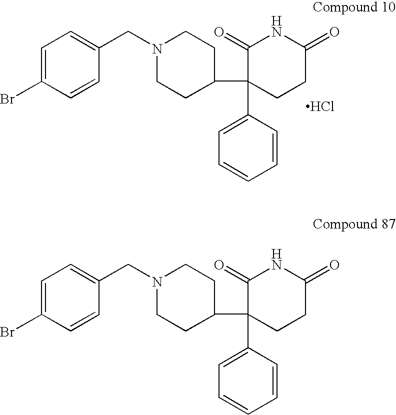

A solution of 1-bromo-4-(bromomethyl)benzene (0.044 mol) in DMF (30 ml) was added dropwise to a mixture of 3-phenyl-[3,4′-bipiperidine]-2,6-dione (0.0367 mol) in Et₃N (25.8 ml) and DMF (125 ml) (moderate exothermic temperature rise). The reaction mixture was stirred for 18 hours at room temperature. Some starting material was left, so extra 1-bromo-4-(bromomethyl)benzene (3.0 g) was added and the resultant reaction mixture was stirred for 44 hours at room temperature. The mixture was poured out into ice-water. DIPE (100 ml) was added and stirring was continued for 15 minutes. The oily precipitate was filtered off, washed with water, and then dissolved in DCM. The organic layer was separated, dried (MgSO₄), filtered and the solvent was evaporated, yielding compound 87. Compound 87 was stirred in boiling 2-propanol (200 ml) and converted into the hydrochloric acid salt (1:1) with 6 N HCl/2-propanol (80 ml, which was added slowly). Stirring at room temperature was continued for 30 minutes. Then, the mixture stood for 2 hours. The precipitate was filtered off, washed with DIPE, and dried (vacuum, 50° C.). Yield: 13.1 g of compound 10 (74.7%).

### k) Preparation of Compound 11

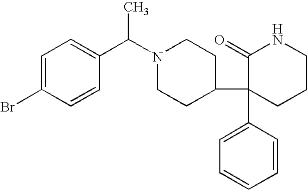

1-Bromo-4-(1-chloroethyl)benzene (0.00025 mol; 0.25 ml of 1 M/DCM) was added to a shaking solution of intermediate 30 (prepared according to A7.c) (0.000174 mol) in Et₃N (0.2 ml) and DMF, p.a. (6 ml). The reaction mixture was shaken further for 18 hours at room temperature, then for one hour at 55° C. The solvent was evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were combined and the solvent was evaporated. Methanol was added and co-evaporated on the rotary evaporator. Yield: 0.051 g of compound 11.

### l) Preparation of Compound 12 and Compound 12a

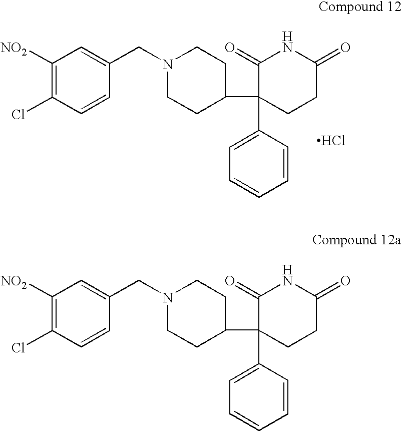

A mixture of 3-phenyl-[3,4′-bipiperidine]-2,6-dione (0.019 mol) and N-ethyl-N-(1-methylethyl)-2-propanamine (0.076 mol) in DMF, p.a. (50 ml) was stirred. A solution of 1-chloro-4-(chloromethyl)-2-nitrobenzene (0.02 mol) in DCM, p.a. (25 ml) was added dropwise and the reaction mixture was stirred at room temperature for 18 hours. Then the mixture was poured in H₂O (300 ml) containing NaHCO₃ (10 g), stirred for 15 minutes and the mixture was extracted with DCM. The separated organic layer was dried (MgSO₄), filtered, and the solvent was evaporated and co-evaporated with toluene. The residue was purified over silica gel on a glass filter (eluent: DCM/CH₃OH 98/2). The product fractions were collected and the solvent was evaporated, yielding compound 12a. The residue was turned into its HCl-salt in CH₃OH with HCl/2-propanol (5 ml; 6 N). The solvents were evaporated and the residue was triturated in Et₂O. The product was filtered off and washed with Et₂O and a small amount of CH₃CH₂OH. The product was dried (vacuum, 50° C.). Yield: 4.2 g. A small part of this fraction (0.2 g) was recrystallised from CH₃CH₂OH (30 ml). The product was filtered off, washed with CH₃CH₂OH and dried (vacuum, 50° C.). Yield: 0.11 g of compound 12.

### Example B2

### a) Preparation of Compound 13

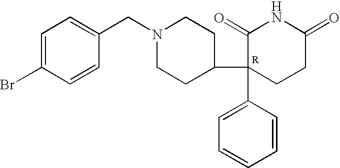

A solution of 1-bromo-4-(chloromethyl)benzene (0.031 mol) in DMF, p.a. (30 ml) was added dropwise to a stirring solution of (R)-3-phenyl-[3,4′-bipiperidine]-2,6-dione (0.0257 mol) and Et₃N (18.1 ml) in DMF, p.a. (100 ml). The reaction mixture was stirred for 65 hours at room temperature. The mixture was poured out into cold water (500 ml) while stirring. The resultant mixture was stirred for 30 minutes. The precipitate was filtered off, washed with plenty of water, then recrystallized from ethanol (180 ml of boiling ethanol), filtered hot, and dried (vacuum, 50° C.). Yield: 8.36 g of compound 13 (73.7%; m.p. (Buchi, visual): 179-180° C.).

### b) Preparation of Compound 14

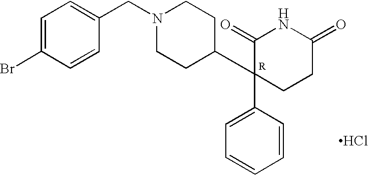

1-Bromo-4-(chloromethyl)benzene (0.0424 mol) was added to a stirring mixture of (R)-3-phenyl-[3,4′-bipiperidine]-2,6-dione (0.0385 mol) and Et₃N (2.7 ml) in DMF, p.a. (30 ml). The reaction mixture was stirred for 18 hours at room temperature. The solvent was evaporated. The residue was stirred in water, filtered off, washed with water and DIPE, and then dried (vacuum, 55° C.). The product was stirred in boiling CH₃CN (30 ml), filtered hot, and the filter residue was rinsed with CH₃CN, then dried (vacuum, 55° C.). Yield: 1.0 g of compound 14.

### Example B3

### a) Preparation of Compound 15

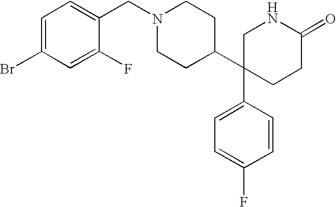

A mixture of intermediate 58 (prepared according to A4A.c) (0.000174 mol) and N-ethyl-N-(1-methylethyl)-2-propanamine (0.2 ml) in DMF p.a. (4 ml) was shaken at room temperature. 4-Bromo-1-(chloromethyl)-2-fluorobenzene (0.0002 mol; 0.2 ml of a 1 M solution in DCM) was added and the resultant reaction mixture was shaken for 18 hours at room temperature. The solvent was evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were collected and the solvent was evaporated. Methanol was added and co-evaporated on the rotary evaporator (2 x). Yield: 0.016 g of compound 15.

### b) Preparation of Compound 16a and Compound 16b

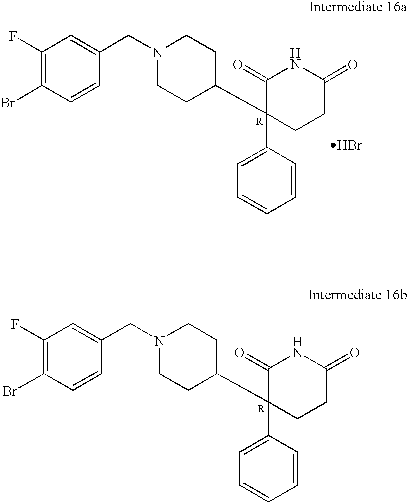

A solution of (R)-3-phenyl-[3,4′-bipiperidine]-2,6-dione (0.000246 mol) and N-ethyl-N-(1-methylethyl)-2-propanamine (0.2 ml) in DMF (6 ml; p.a.) was stirred. 1-Bromo-4-(bromomethyl)-2-fluorobenzene (0.0003 mol) was added and the reaction mixture was stirred at room temperature for 4 days. The solvent was evaporated and the residue was stirred in H₂O. This mixture was extracted with DCM/CH₃OH 90/10. The separated organic layer was dried (MgSO₄), filtered, and the solvent was evaporated. The residue was stirred in DCM/CH₃OH 90/10 (1.5 ml), filtered off, washed and dried (vacuum, 50° C.). Yield: 0.0074 g of compound 16a. The filtrate was purified by Flash Tube (eluent: DCM/CH₃OH 90/10). The product fraction was isolated and stirred in DCM/CH₃OH 90/10. The silica was filtered off and washed with DCM/CH₃OH 90/10. The solvent was evaporated. Yield: 0.027 g of compound 16b.

### c) Preparation of Compound 17 and Compound 18

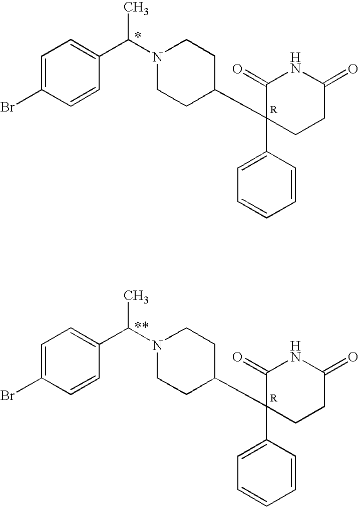

A mixture of (R)-3-phenyl-[3,4′-bipiperidine]-2,6-dione (0.00092 mol) and N-ethyl-N-(1-methylethyl)-2-propanamine (0.00276 mol) in DMF, p.a. (25 ml) was stirred at room temperature. 1-Bromo-4-(1-bromoethyl)benzene (0.001 mol) was added and the reaction mixture was stirred for 18 hours at room temperature. The solvent was evaporated. 2-Propanol was added and co-evaporated. The residue was stirred in a half-saturated aqueous NaHCO₃ solution and the product was extracted with DCM. The organic layer was separated, dried (MgSO₄), filtered and the solvent was evaporated. The residue was triturated in DIPE, and the resulting precipitate was filtered off, washed and dried (vacuum, 55° C.). Yield: 0.22 g. This fraction was separated into its enantiomers by chiral column chromatography over an AD column (eluent: 100% ethanol). Two product fraction groups were collected and their solvent was evaporated to give residue A for the first eluted fraction and B for the second fraction. Residue A was stirred in DIPE, filtered off, washed and dried (vacuum, 55° C.). Yield: 0.011 g of compound 18 (R,**). Residue B was stirred in methanol, filtered off, washed and dried (vacuum, 55° C.). Yield: 0.055 g of compound 17 (R,*).

(For compounds 17 and 18, the absolute configuration of the second chiral centre was not determined. * means that it can be R or S, ** means that it can be S or R, thus if * is R then ** is S; if * is S then ** is R.)

### d) Preparation of Compound 192

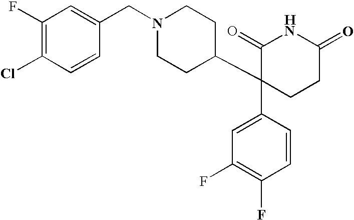

1-Chloro-4-(chloromethyl)-2-fluorobenzene (0.0002 mol; 1 M in DCM) was added to a shaking mixture of intermediate 73 (0.0002 mol) (see A16e) in N-ethyl-N-(1-methylethyl)-2-propanamine (0.0024 mol) and DMF (3 ml; p.a.). The reaction mixture was shaken at room temperature for 68 hours. The solvent was evaporated. The residue was purified by reversed phase high performance liquid chromatography (method A). The desired product fractions were collected and the solvent was evaporated, and co-evaporated 2 times with CH₃OH, yielding compound 192.

### Example B4

### a) Preparation of Compounds 19, 19a, 21, and 21a

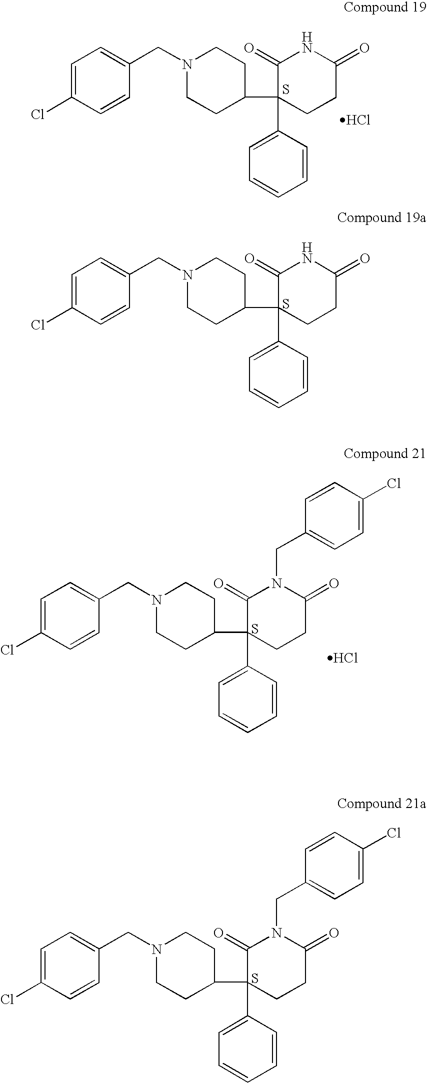

A mixture of (S)-3-phenyl-[3,4′-bipiperidine]-2,6-dione (0.0018 mol), 1-chloro-4-(chloromethyl)benzene (0.0025 mol) and K₂CO₃ (0.5 g) in DMF (25 ml) was stirred for 4 hours at 60° C. The solvent was evaporated. The residue was purified by reversed-phase HPLC. The product fractions were collected and worker up. The residue was dried (vacuum, overnight), yielding compound 19a. Compound 19a was converted into the hydrochloric acid salt (1:1) with HCl/2-propanol, filtered off and dried. Yield: 0.0429 g of compound 19 (5.5%).

Column fractions containing another compound were collected and the solvent was evaporated, yielding compound 21a. The free base was converted into the hydrochloric acid salt (1:1) with HCl/2-propanol, filtered off and dried. Yield: 0.287 g of compound 21.

### b) Preparation of Compounds 20, 20a, 189 and 189a

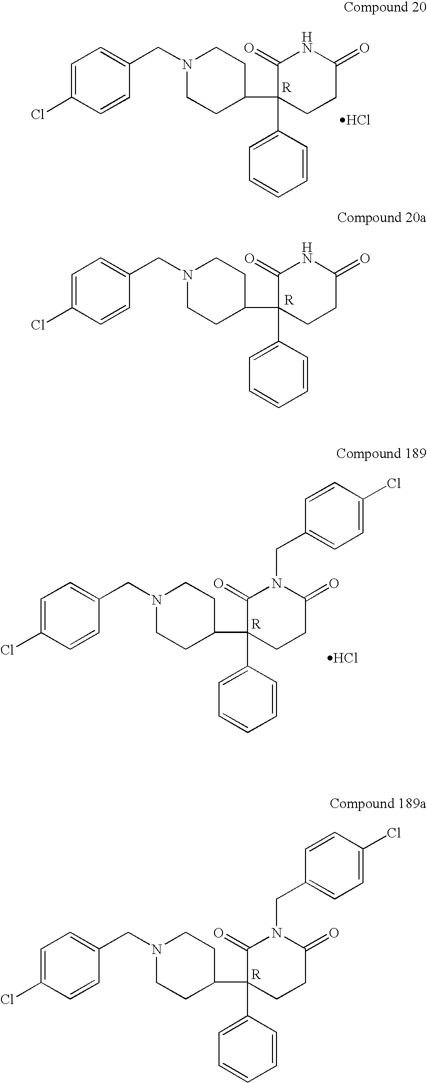

Compounds 20, 20a, 189, 189a were prepared according to the procedure described for compounds 19, 19a, 21, 21a but starting from (R)-3-phenyl-[3,4′-bipiperidine]-2,6-dione (0.0018 mol). Yield: 0.032 g of compound 20; 0.164 g of compound 189.

### Example B5

### Preparation of Compound 22

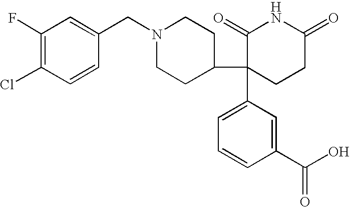

A mixture of intermediate 52 (prepared according to A1A.e) (0.00015 mol) and N-ethyl-N-(1-methylethyl)-2-propanamine (0.2 ml) in DMF, p.a. (5 ml) was shaken at room temperature. 1-Chloro-4-(chloromethyl)-2-fluorobenzene (0.0002 mol; 0.2 ml of 1 M solution in DCM) was added and the resultant reaction mixture was shaken for 18 hours at room temperature. NaHCO₃, half saturated aqueous solution (2 ml) was added and the reaction mixture was shaken for 24 hours at room temperature. The solvents were evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The desired fractions were collected and the solvent was evaporated until a ±3-ml-concentrate remained. The solid was filtered off, washed with water and dried (vacuum, 50° C.), yielding compound 22.

### Example B6

### Preparation of Compound 23

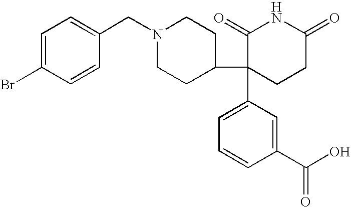

A solution of intermediate 39 (prepared according to A10.d) (0.0021 mol) in acetic acid (10 ml) was stirred. H₂SO₄ (2.5 ml) was added and the reaction mixture was stirred and refluxed at 165° C. for 100 minutes. The mixture was allowed to cool to room temperature. The mixture was poured out into ice-water containing 10 ml NaOH 50%. The mixture was continued stirring for 20 minutes. The precipitate was filtered off and dried (vacuum, 60° C.). Yield: 0.53 g of fraction A (52%). The filtrate was neutralized with NaHCO₃ (pH=about 7) and DCM was added (20 ml). The mixture was stirred for 30 minutes. The precipitate was filtered off, washed with H₂O and dried (vacuum, 60° C.). This fraction was combined with fraction A and the product (0.82 g) was purified by high-performance liquid chromatography (NH₄OAc). The product fractions were collected and partially evaporated till ±80 ml. The concentrate was left standing for 18 hours and the formed precipitate was filtered off, washed with H₂O and dried (vacuum, 55° C.). Yield: 0.33 g of compound 23 (32.4%).

### Example B7

### Preparation of Compound 24 and Compound 25

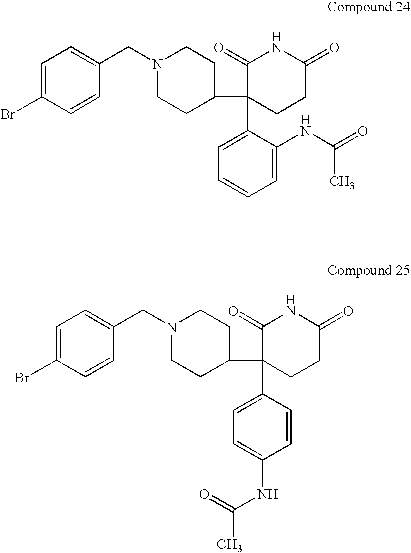

A suspension of intermediate 53 (prepared according to A12) (0.000698 mol) and THF (5 ml) was heated while CH₃OH (2 ml) was added dropwise. The obtained homogeneous solution was cooled to room temperature. Then N-ethyl-N-(1-methylethyl)-2-propanamine (0.001047 mol) and 1-bromo-4-(chloromethyl)benzene (0.000733 mol) were added and the mixture was stirred overnight at room temperature. The reaction mixture was heated for 1 hour at 60° C. The solvent was evaporated and a residue was obtained. Yield: 0.249 g. The residue was purified by reversed-phase high-performance liquid chromatography (NH₄OAc/CH₃OH/CH₃CN gradient). Two product fractions were isolated, the solvents were evaporated and the products were extracted with H₂O/EtOAc. The separated EtOAc layers were dried (MgSO₄), filtered and the solvents were evaporated. Yield: 0.035 g of fraction A (para substituted). Yield: 0.015 g of fraction B (ortho substituted). The 2 fractions were purified again by reversed-phase high-performance liquid chromatography (NH₄HCO₃ gradient). Yield: 0.022 g of compound 25 (6.3%; para substituted). Yield: 0.007 g of compound 24 (2%; ortho substituted).

### Example B8

### a) Preparation of Compound 26

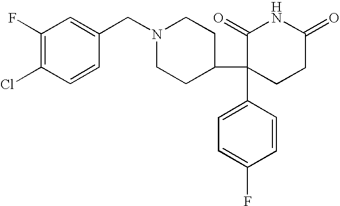

A mixture of 2-(p-fluorophenyl)-2-(4-piperidyl)-glutarimide (0.000128 mol), 4-chloro-3-fluorobenzaldehyde (0.00022 mol) and NaBH(OAc)₃ (0.00064 mol) in DCM, p.a. (8 ml) was shaken at room temperature. Acetic acid, p.a. (0.00022 mol) was added. The reaction mixture was stirred for 18 hours at room temperature. The solvent was evaporated. The residue was purified by reversed-phase high-performance liquid chromatography (Column: Xterra Prep MS C18, Length: 10 cm, I.D.: 19 mm, particle size: 5 μm; eluent: (NH₄OAc in H₂O)/CH₃OH/CH₃CN gradient). The desired fractions were combined, collected and the solvent was evaporated. Yield: fraction A (still a salt because of the NH₄OAc used in the chromatography). This fraction was stirred vigorously for 2 hours in a biphasic mixture of DCM (4 ml) and a saturated aqueous NaHCO₃ solution (1 ml). The mixture was filtered and dried through an Isolute HM-N filter and the filtrate was evaporated. Yield: 0.0423 g of compound 26.

### b) Preparation of Compound 27

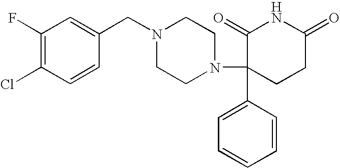

A mixture intermediate 21 (prepared according to A5.e) (0.000164 mol), 4-chloro-3-fluorobenzaldehyde (0.000197 mol) and NaBH(OAc)₃ (0.00049 mol) in DCM, p.a. (6 ml) was stirred at room temperature. Acetic acid, p.a. (0.000197 mol) was added. The reaction mixture was stirred for 18 hours at room temperature. More 4-chloro-3-fluorobenzaldehyde (0.2 ml) and acetic acid, p.a. (0.015 ml) were added and the reaction mixture was stirred for 24 hours at room temperature. The solvents were evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The desired fractions were combined, collected and the solvent was evaporated. Methanol was added and co-evaporated. Yield: 0.0375 g of compound 27.

### c) Preparation of Compound 28

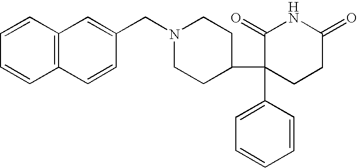

A mixture of 3-phenyl-[3,4′-piperidine]-2,6-dione (0.000191 mol), 2-naphthalenecarboxaldehyde (0.000191 mol) and NaBH(OAc)₃ (0.00057 mol) in DCM, p.a. (8 ml) was stirred at room temperature. Acetic acid (0.000191 mol) was added. The reaction mixture was stirred over the weekend at room temperature, then stood for 7 days at room temperature. 6 N HCl/2-propanol (3 drops) were added. The solvent was evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The desired fractions were combined, collected and the solvent was evaporated. Methanol was added and co-evaporated. Yield: 0.031 g of compound 28 (39.3%).

### d) Preparation of Compound 29

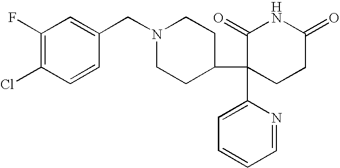

A mixture of intermediate

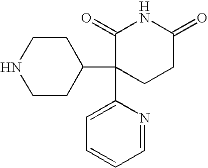

(prepared according to A1.f) (0.000227 mol), 4-chloro-3-fluorobenzaldehyde (0.00034 mol) and NaBH(OAc)₃ (0.00068 mol) in DCM p.a. (4 ml) was stirred. Acetic acid (0.00034 mol) was added and the reaction mixture was stirred at room temperature for 3 days. HCl (2 ml; 1 N) was added and stirring was continued for 1 hour. Then NaHCO₃ was added till pH≧8 (foaming). The separated water layer was extracted with DCM/CH₃OH 98/2. The combined organic layers were dried (MgSO₄), filtered and the solvent was evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were combined, the solvent was evaporated and two times co-evaporated with CH₃OH. Yield: 0.027 g of compound 29 (28.5%).

### e) Preparation of Compound 30

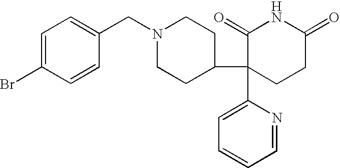

A mixture of intermediate

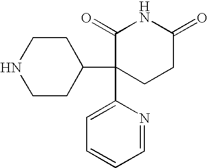

(prepared according to A1.f) (0.000227 mol), 4-bromobenzaldehyde (0.00034 mol) and NaBH(OAc)₃ (0.00068 mol) in DCM p.a. (4 ml) was stirred. Acetic acid (0.00034 mol) was added and the reaction mixture was stirred at room temperature for 3 days. HCl (2 ml; 1N) was added and stirring was continued for 1 hour. Then NaHCO₃ was added till pH≧8 (foaming). The separated water layer was extracted with DCM/CH₃OH 98/2. The combined organic layers were dried with MgSO₄, filtered and the solvent was evaporated. The residue was purified by Flash Tube (eluent: DCM/CH₃OH 90/10). The product fractions were collected, stirred in DCM/CH₃OH 90/10, filtered off to remove the silica and the solvent was evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were combined, the solvent was evaporated and two times co-evaporated with CH₃OH. Yield: 0.0247 g of compound 30 (24.5%).

### f) Preparation of Compound 31

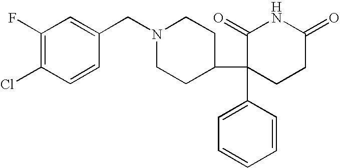

A mixture of 3-phenyl-[3,4′-bipiperidine]-2,6-dione (0.00019 mol), 4-chloro-3-fluorobenzaldehyde (0.0002 mol) and NaBH(OAc)₃ (0.121 g) in DCM p.a. (8 ml) was shaken at room temperature. Acetic acid (0.0002 mol) was added. The reaction mixture was shaken for 40 hours at room temperature, then quenched with 2 drops of 6N HCl/2-propanol. The solvents were evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were collected and the solvent was evaporated. Methanol was added and co-evaporated. Yield: 0.004 g of compound 31 (5%).

### g) Preparation of Compound 76

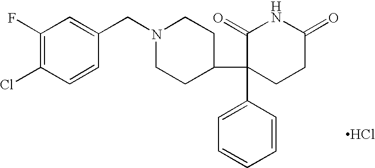

A mixture of 3-phenyl-[3,4′-bipiperidine]-2,6-dione (0.03 mol), 4-chloro-3-fluorobenzaldehyde (0.032 mol) and NaBH(OAc)₃ (0.09 mol) in DCM p.a. (200 ml) was stirred at room temperature. Acetic acid, p.a. (0.032 mol) was added slowly. The reaction mixture was stirred for 65 hours at room temperature. Water (150 ml) was added. The organic layer was separated, dried (MgSO₄), filtered and the solvent was evaporated. The residue was purified over silica gel on a glass filter (eluent: DCM/CH₃OH 98/2). The desired fractions were collected and the solvent was evaporated. The residue was dissolved in hot 2-propanol (100 ml) and converted into the hydrochloric acid salt (1:1) with 6 N HCl/2-propanol (13 ml). The precipitate was filtered off (at room temperature), washed with 2-propanol, and dried (vacuum, 60° C., over the weekend). Yield: 11.7 g of compound 76 (86.4%).

### Example B9

### a) Preparation of Compound 32

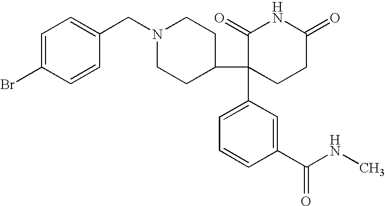

A mixture of intermediate 54 (prepared according to A13) (0.000113 mol) in CH₃CH₂OH/CH₃NH₂ 8M (2.5 ml) was stirred at room temperature for 20 hours. The solvent was evaporated, the residue was dissolved in acetic acid (2 ml) and H₂SO₄ (0.1 ml) was added. The solution was heated to 165° C. in a microwave for 10 minutes. Then the solution was poured into 20 ml H₂O and NaHCO₃ was added in portions till pH 8-9. The product was extracted two times with DCM/CH₃OH 95/5. The combined organic layers were dried (MgSO₄), filtered and the solvent was evaporated. Yield: 0.042 g of compound 32 (74.5%).

### b) Preparation of Compound 33

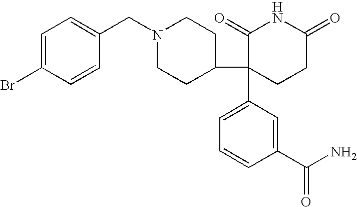

A mixture of intermediate 54 (prepared according to A13)(0.00065 mol) in CH₃OH/NH₃ 7N (25 ml) was stirred. The reaction mixture was stirred further for 21 hours and became a solution. The solvent was evaporated. The residue was stirred in H₂O and then NaHCO₃ was added till alkaline pH. Then DCM/CH₃OH (10 ml) was added and the mixture was stirred for 18 hours. The precipitate was filtered off, washed with H₂O and DCM, and dried (vacuum, 60° C.). Yield: 0.078 g of compound 33 (24.5%).

### c) Preparation of Compound 34

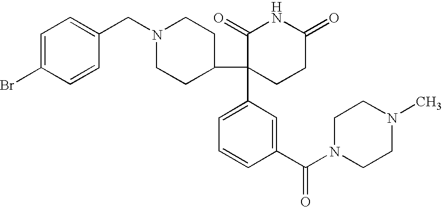

A mixture of intermediate 54 (prepared according to A13) in 1,4-dioxane (p.a.; dried on molecular sieves; 3 ml) was stirred. 1-Methylpiperazine (0.1 ml) was added and the reaction mixture was stirred at room temperature for 18 hours. The solvent was evaporated. The residue was stirred in DCM, washed with H₂O, dried (MgSO₄), filtered and the solvent was evaporated. The residue was purified by high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were collected, the solvent was evaporated and two times co-evaporated with CH₃OH. Yield: 0.0365 g of compound 34.

### d) Preparation of Compound 91

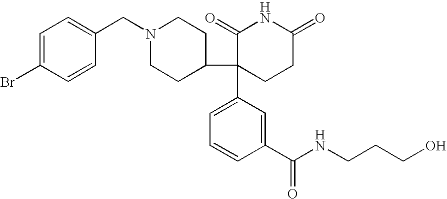

A mixture of intermediate 54 (prepared according to A13) (0.0001 mol) in 1,4-dioxane (p.a.; dried on molecular sieves; 3 ml) was stirred. 3-Amino-1-propanol (0.2 ml) was added and the solution was stirred at room temperature for 3 hours. The solution was poured into H₂O (20 ml), and DCM (10 ml) was added. Then a saturated aqueous NH₄Cl solution was added till a clean bi-phasic solution was formed. The separated organic layer was dried (MgSO₄), filtered and the solvent was evaporated. The residue was purified by high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were collected, the solvent was evaporated and co-evaporated with CH₃OH. Yield: 0.0305 g of compound 91.

### Example B10

### Preparation of Compound 35

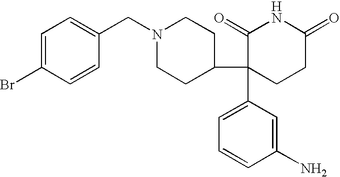

A solution of intermediate 55 (prepaid according to A14)(0.0001 mol) and CF₃COOH (0.2 ml) in DCM (2 ml) was stirred at room temperature for 18 hours. The solvent was evaporated and the residue was stirred in a saturated aqueous NaHCO₃ solution. The solution was extracted twice with DCM/CH₃OH 90/10. The combined organic layers were dried (MgSO₄), filtered, and the solvent was evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The product fractions were combined, the solvent was evaporated and two times co-evaporated with CH₃OH. Yield: 0.010 g of compound 35 (21.9%).

### Example B11

### Preparation of Compound 36

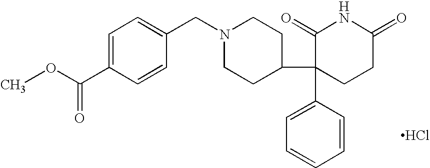

Intermediate

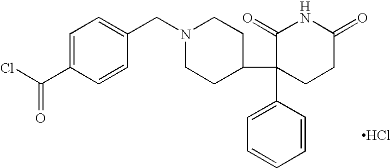

(prepared according to A13) (max. 0.00011 mol) was kept under N₂ atmosphere. CH₃OH p.a. (2 ml) was added. The mixture was stirred for 5 minutes. The solvent was evaporated. Yield: 0.0488 g of compound 36.

### Example B12

### Preparation of Compound 37

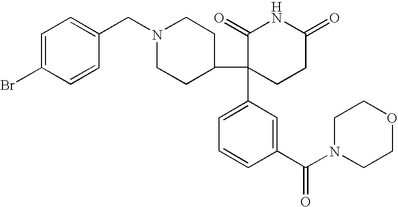

A mixture of compound 23 (prepared according to B6) (0.000115 mol) in DCM, p.a. (10 ml) was stirred. 1,1′-Carbonylbis-1H-imidazole (0.00015 mol) was added and the reaction mixture was stirred at room temperature for 18 hours. Then Et₃N (1 ml) was added and stirring of the reaction mixture was continued at room temperature for 3 hours (solution after 30 minutes). Then a second amount of 1,1′-carbonylbis-1H-imidazole (0.000154 mol) was added and the reaction mixture was stirred further at room temperature for 20 hours. Then an extra amount of fresh 111′-carbonylbis-1H-imidazole (0.000154 mol) was added and the reaction mixture was stirred at room temperature for 20 hours again. Morpholine (1 ml) was added and the solvent was evaporated. The residue was purified by high-performance liquid chromatography (Column: Xterra Prep MS C18, Length: 10 cm, I.D.: 19 mm, particle size: 5 μm; eluent: (0.5% NH₄OAc in H₂O)/CH₃OH/CH₃CN gradient). The product fractions were collected and the solvent was evaporated. Yield: 0.0443 g of residue (69.5%; contains salts). The residue was stirred in a biphasic solution of DCM (5 ml) and NaHCO₃ (1 ml of an aqueous saturated solution). The biphasic solution was filtered and dried over an ISOLUTE HM-N filter. The filtrate was evaporated. Yield: 0.0263 g of compound 37 (41.2%).

### Example B13

### Preparation of Compound 38

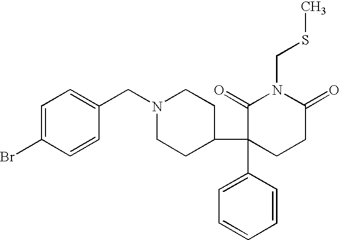

K₂CO₃ (0.000356 mol), then bromo(methylthio)methane (0.000214 mol) was added to a solution of compound 10 (prepared according to B1.j) (0.000178 mol) in DMF, p.a. (4 ml), stirred at room temperature. The reaction mixture was stirred for 7 hours at 70° C. More bromo(methylthio)methane (0.02 ml) was added and the reaction mixture was stirred for 18 hours at 70° C. The mixture was allowed to cool to room temperature. Water was added. This mixture was extracted with diethyl ether. The ether extracts were evaporated. The residue was purified by reversed-phase high-performance liquid chromatography (Column: Xterra Prep MS C18, Length: 10 cm, I.D.: 19 mm, particle size: 5 μm; eluent: (0.2% NH₄HCO₃ in H₂O)/CH₃OH/CH₃CN gradient). The desired fractions were collected and the solvent was evaporated. Methanol was added then co-evaporated. Yield: 0.006 g of compound 38.

### Example B14

### Preparation of Compound 39

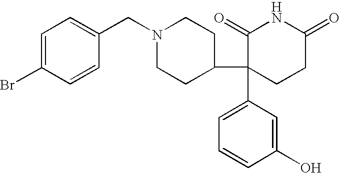

A solution of compound 8 (prepared according to B1.h) (0.000127 mol) in DCM, p.a. (3 ml) was stirred on an ice-bath at 0° C. BBr₃ in DCM (1 M) (0.4 ml) was added and the reaction mixture was first stirred at 0° C. for 1 hour and then at room temperature for 3 hours. An extra amount of BBr₃ in DCM (1 M) (0.2 ml) was added while the reaction mixture was cooled to 0° C. The mixture was stirred at 0° C. for 1 hour and then at room temperature for 18 hours. Then CH₃OH (3 ml) was added which resulted in a solution and stirring was continued for 30 minutes. The solvents were evaporated. The residue was stirred in 2 ml H₂O and NaHCO₃ (1.5 ml) was added. The product was extracted two times with DCM. The combined organic layers were dried (MgSO₄), filtered, and the solvent was evaporated. The residue was purified over a Flash Tube (eluent: DCM/CH₃OH 85/15). The product fraction was isolated, stirred in DCM/CH₃OH 90/10, filtered, washed and the filtrate was evaporated. Yield: 0.022 g of compound 39 (37.8%).

### Example B15

### Preparation of Compound 40 and Compound 41

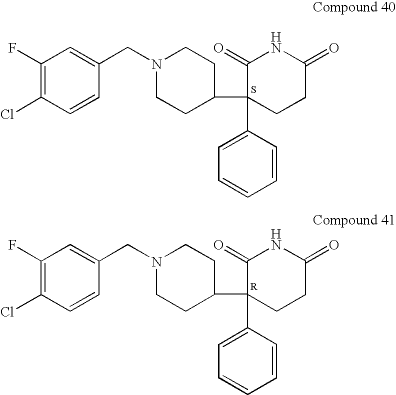

A mixture of compound 31 (prepared according to B8.f) (0.00553 mol) in NaHCO₃, saturated aqueous solution (100 ml) and DCM (150 ml) was stirred vigorously for 2 hours at room temperature. The organic layer was separated, dried (MgSO₄), filtered and the solvent was evaporated. Methanol was added and co-evaporated. Yield: 2.3 g (free base of racemic mixture). This fraction was separated into its enantiomers by chiral column chromatography over an OJ column (eluent: 100% CH₃OH; 80 ml/min). Two product fraction groups were collected and their solvent was evaporated. Each residue (white foam; ±1.1 g each) was crystallized from 2-propanol (15 ml). Each precipitate was filtered off, washed with 2-propanol and dried (vacuum, 60° C.). Yield: 0.90 g of compound 40 (S enantiomer) and 0.74 g of compound 41 (R-enantiomer).

### Example B16

### Preparation of Compound 42

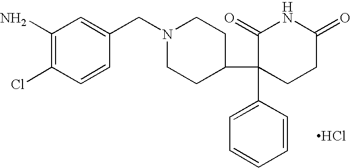

A solution of compound 12 (prepared according to B1.1) (0.0088 mol) in CH₃OH, p.a. (100 ml) was hydrogenated with Pt/C₅% (2 g) as a catalyst in the presence of thiophene solution in CH₃OH (1 ml). After uptake of H₂ (3 equiv), the catalyst was filtered off and the filtrate was evaporated. The residue was stirred in Et₂O, filtered off, washed with Et₂O and dried (vacuum, 50° C.). Yield: 3.15 g of compound 42 (86.9%).

### Example B17

### a. Preparation of Compound 185 and Compound 186

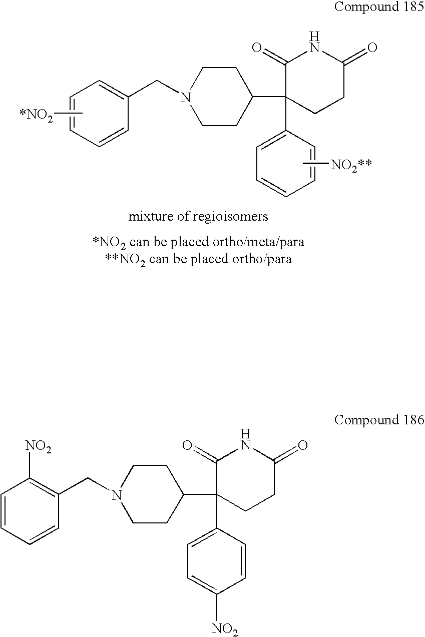

A beaker containing HNO₃, fuming (0.196 mol) was cooled in an ice/salt bath to −9° C. 3-Phenyl-1′-(phenylmethyl)-[3,4′-bipiperidine]-2,6-dione (0.00276 mol) was added in small portions in a way that the temperature did not exceed 0° C. After the addition, the reaction mixture was stirred for 17 minutes and was then poured gradually on ice with Na₂CO₃ (0.142 mol) and a small quantity of DCM. An extra amount of Na₂CO₃ was added to make the water layer alkaline. The mixture was extracted with DCM (3×). The separated organic layer was dried (MgSO₄), filtered and the solvent was evaporated. Yield: 1.124 g of residue. The residue was purified by reversed phase high-performance liquid chromatography (NH₄HCO₃). The purification resulted in 2 different product fractions: (I) en (II). The solvents of both fractions were evaporated and co-evaporated with CH₃OH/CH₃CN. Fraction (I) resulted in 0.757 g of compound 185 (60.6%; mixture of regio-isomers). Fraction (II) resulted in 0.051 g of compound 186 (4.1%).

### b. Preparation of compound 187

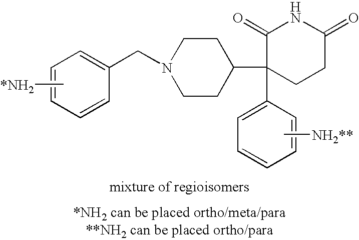

A suspension of compound 185 (0.00574 mol) in THF (100 ml) was hydrogenated at room temperature for 12 hours with Pt/C₅% (1 g) as a catalyst in the presence of V₂O₅ (0.1 g) and thiophene solution (1 ml) and then at 50° C. for 12 hours. After uptake of H₂, the catalyst was filtered off and the filtrate was evaporated. Yield: 2.355 g of compound 187 (100%).

### c. Preparation of Compound 188

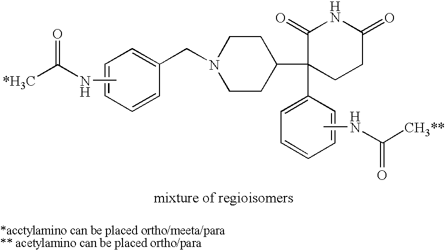

A solution of compound 187 (0.00298 mol) and N-ethyl-N-(1-methylethyl)-2-propanamine (0.00626 mol) in DCM, p.a. (50 ml) was stirred. Acetic acid anhydride (0.00611 mol) was added and the mixture was stirred overnight at room temperature. An aqueous Na₂CO₃ solution (10%) was added and the DCM layer was separated. The water layer was extracted a second time with DCM and the organic layers were combined. Some precipitation at the sides of the separation flask was observed. The water layer was extracted with EtOAc and this layer was also isolated. After removal of the solvents, the precipitate at the sides of the flask was partially extracted in CH₃OH. The extraction with CH₃OH was not improved by adding small amounts of CH₃CN or DCM. The separated DCM layer was dried (MgSO₄), filtered, the solvent was evaporated and an oily residue was obtained. Yield: 0.837 g of fraction A. The EtOAc layer was also dried (MgSO₄), filtered and the solvent was evaporated. Yield: 0.733 g of fraction B. The solvents of the CH₃OH/CH₃CN/DCM mixture were also removed. Yield: 0.484 g of fraction C. Fraction B and fraction C were combined in THF and the solvent was evaporated. Yield: 0.991 g of compound 188 (69.7%).

### Example B18

### Preparation of Compound 193

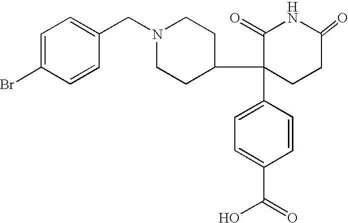

1-Bromo-4-(chloromethyl)benzene (0.18 ml, 0.0002 mol, 1 M solution in DCM) was added to a shaking solution of intermediate 68 (0.042 g, 0.0001 mol) (see A15e) in H₂O (2 ml), N-ethyl-N-(1-methylethyl)-2-propanamine (0.2 ml) and DMF (4 ml). The reaction mixture was shaken at room temperature for 18 hours. The solvent was evaporated. The residue was purified by reversed phase high performance liquid chromatography (method A). The desired fractions were collected and the solvent was evaporated, and co-evaporated with CH₃OH. Yield: 0.008 g of compound 193.

### Example B19

### Preparation of Compound 194

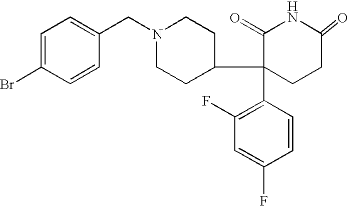

A solution of intermediate 44 (0.00019 mol) (see A11e) in N-ethyl-N-(1-methylethyl)-2-propanamine (0.25 ml), DMF (3.5 ml, p.a.) and DCM (0.5 ml) was shaken. 1-Bromo-4-(chloromethyl)benzene (0.00022 mol; 0.22 ml of a 1 M solution in DCM) was added and the reaction mixture was shaken further for 18 hours. The solvents were evaporated and the residue was purified by reversed-phase high performance liquid chromatography (method A). The product fractions were collected and the solvent was evaporated and co-evaporated with CH₃OH, yielding compound 194.

### Example B20

### a. Preparation of Compound 203

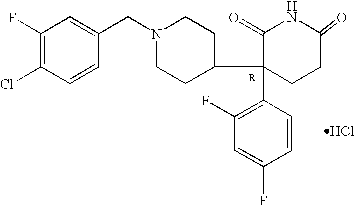

CH₃COOH (0.22 ml, 0.0037 mol) was added to a stirring mixture of intermediate 63 (0.75 g, 0.0024 mol) (see A11h), 4-chloro-3-fluorobenzaldehyde (0.578 g, 0.0037 mol), sodium triacetoxyborohydride (1.55 g, 0.0073 mol) and DCM (20 ml, p.a.). The reaction mixture was stirred at room temperature for 18 hours. An aqueous HCl solution (1 N, 10 ml) was added to the reaction mixture and then stirred vigorously for 25 minutes. The reaction mixture was left standing for 24 hours. The precipitate was filtered off, washed with DCM and H₂O and dried (vacuum, 55° C.). The residue was recrystallized from H₂O/HCl (35 ml/(1 ml, 1 N)). The precipitate was filtered off hot, washed with H₂O and dried (vacuum, 55° C.). The residue was recrystallized from EtOH (35 ml). The precipitate was filtered off, washed with EtOH and dried (vacuum, 55° C.). Yield: 0.455 g of compound 203 (38.4%).

### b. Preparation of Compound 204

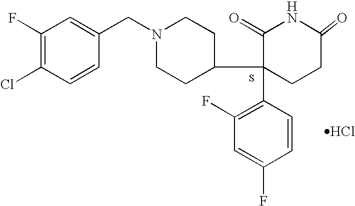

CH₃COOH (0.25 ml) was added to a stirring mixture of intermediate 62 (0.84 g, 0.0027 mol) (see A11g), 4-chloro-3-fluorobenzaldehyde (0.65 g, 0.0041 mol), sodium triacetoxyborohydride (1.73 g, 0.0082 mol) and DCM (20 ml; p.a.). The reaction mixture was stirred at room temperature for 18 hours. An aqueous HCl solution (1 N, 10 ml) was added to the reaction mixture and then stirred for 20 minutes. The precipitate was filtered off, washed with DCM and H₂O and dried (vacuum, 50° C.). The residue was recrystallized from H₂O/HCl (30 ml/1 ml). The precipitate was filtered off hot, washed with H₂O and dried (vacuum, 55° C.). Yield: 0.765 g of compound 204 (57.7%).

### c. Preparation of Compound 195

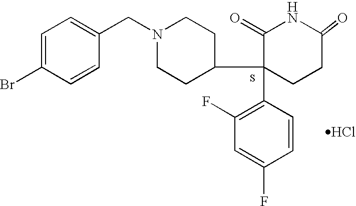

CH₃COOH (0.2 ml) was added to a stirring mixture of intermediate 62 (0.68 g, 0.0022 mol) (see A11g), 4-bromobenzaldehyde (0.61 g, 0.0033 mol), sodium triacetoxyborohydride (1.4 g, 0.0066 mol) and DCM (15 ml; p.a.) and was then stirred at room temperature for 18 hours. An aqueous HCl solution (1 N, 10 ml) was added and the reaction mixture was stirred for 1 hour. Then NaHCO₃ was added portionwise to the mixture until pH=8 to 9. The layers were separated and the separated organic layer was washed with H₂O, dried (MgSO₄), filtered and the filtrate's solvent was evaporated, and co-evaporated with 2-propanol. The residue was stirred in HCl/2-propanol (6 N). The precipitate was filtered off, washed with 2-propanol and dried (vacuum, 55° C.). Yield: 1.00 g of compound 195.

### Example B21

### Preparation of Compound 209

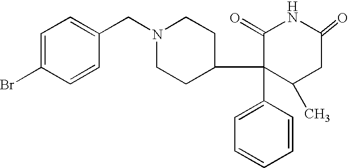

1-Bromo-4-(chloromethyl)benzene (0.00018 mol, 0.18 ml) was added to a stirring mixture of intermediate 76 (0.000167 mol, 0.048 g) (see A17c), N-ethyl-N-(1-methylethyl)-2-propanamine (0.00115 mol, 0.2 ml) and DMF (4 ml; p.a.). The reaction mixture was stirred further at room temperature for 18 hours. The solvents were evaporated. The residue was stirred in DCM/CH₃OH (1.5 ml, 90/10) and filtered over an Acrodisc filter. The filtrate was purified by flash column chromatography (DCM/CH₃OH 90/10) over silica gel. The desired fraction was collected and the solvent was evaporated. Yield: 0.0203 g of compound 209 (26.7%).

### Example B22

### Preparation of Compound 210

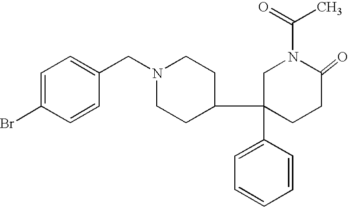

A mixture of compound 5 (0.00037 mol, 0.16 g) and acetyl acetate (2.5 ml) was reacted in a sealed tube at 100° C. over 1 hour. Then the reaction mixture was allowed to reach room temperature. The solvent was evaporated. The residue was filtered over a RediSEP Cartridge (DCM/CH₃OH from 100/0 till 99/1 till 98/2 till 97/3). The desired fractions were combined and evaporated. Yield: 0.037 g of compound 210 (21.3%). Table 1 to 7 list the compounds of formula (I) which were prepared according to one of the above examples (Ex. No.)

TABLE 1

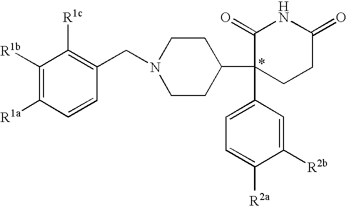

| | | | | | | | Stereochem./ |
|---|---|---|---|---|---|---|---|
| Co. No. | Exp. No. | R^{1a} | R^{1b} | R^{1c} | R^{2a} | R^{2b} | Salt |
| 43 | B4.a | —F | H | H | H | H | *S; •HCl (1:1) |
| | | | | | | | •ethanolate |
| | | | | | | | (1:1) |
| 44 | B1.a | —F | H | H | H | H | *RS |
| 45 | B1.a | —F | H | H | H | H | *RS; |
| | | | | | | | •HCl (1:1) |
| 46 | B8.f | —F | H | —OCH₃ | H | H | *RS |
| 47 | B1.a | —F | —F | H | H | H | *RS |
| 48 | B1.c | —F | —F | H | H | —Cl | *RS |
| 49 | B1.c | —F | —F | H | —F | H | *RS |
| 50 | B8.f | —F | —F | —F | H | H | *RS |
| 51 | B8.a | —F | —F | —F | H | —Cl | *RS |
| 52 | B8.a | —F | —F | —F | H | —CH₃ | *RS |
| 53 | B8.a | —F | —F | —F | H | —OCH₃ | *RS |
| 54 | B8.a | —F | —F | —F | —F | H | *RS |
| 55 | B1.a | —F | —Cl | H | H | H | *RS |
| 56 | B1.c | —F | —Cl | H | —F | H | *RS |
| 190 | B18 | —F | —Cl | —H | —COOH | —H | *RS |
| 57 | B8.f | —F | —Br | H | H | H | *RS |
| 58 | B8.a | —F | —Br | H | —F | H | *RS |
| 59 | B8.f | —F | —CH₃ | H | H | H | *RS |
| 60 | B8.f | —F | —OCH₃ | H | H | H | *RS |
| 61 | B1.a | —Cl | H | H | H | H | *RS |
| 62 | B6 | —Cl | H | H | H | H | *RS; HCl |
| 20 | B4.b | —Cl | H | H | H | H | *R; •HCl |
| 20a | B4.b | —Cl | H | H | H | H | |
| 19 | B4.a | —Cl | H | H | H | H | *S; •HCl |
| 19a | B4.a | —Cl | H | H | H | H | |
| 63 | B1.c | —Cl | H | H | H | —Cl | |
| 64 | B1.a | —Cl | H | H | H | —CH₃ | *RS |
| 65 | B1.c | —Cl | H | H | H | —CF₃ | *RS |
| 66 | B5 | —Cl | H | H | H | —COOH | *RS |
| 67 | B1.h | —Cl | H | H | H | —OCH₃ | *RS |
| 68 | B1.c | —Cl | H | H | —F | H | *RS |
| 69 | B1.h | —Cl | H | H | —OCH₃ | H | *RS |
| 191 | B18 | —Cl | —H | —H | —COOH | —H | *RS |
| 70 | B1.a | —Cl | H | —Cl | H | H | *RS |
| 71 | B1.c | —Cl | H | —Cl | H | —Cl | *RS |
| 72 | B1.a | —Cl | H | —Cl | H | —CH₃ | *RS |
| 73 | B5 | —Cl | H | —Cl | H | —COOH | *RS |
| 74 | B1.a | —Cl | H | —Cl | H | —OCH₃ | *RS |
| 75 | B1.c | —Cl | H | —Cl | —F | H | *RS |
| 31 | B8.f | —Cl | —F | H | H | H | *RS |
| 76 | B8.g | —Cl | —F | H | H | H | *RS; •HCl |
| 40 | B15 | —Cl | —F | H | H | H | *S; (+) |
| 41 | B15 | —Cl | —F | H | H | H | *R; (−) |
| 77 | B8.a | —Cl | —F | H | H | —Cl | |
| 78 | B8.a | —Cl | —F | H | H | —CH₃ | *RS |
| 22 | B5 | —Cl | —F | H | H | —COOH | *RS |
| 79 | B8.a | —Cl | —F | H | H | —OCH₃ | *RS |
| 26 | B8.a | —Cl | —F | H | —F | H | *RS |
| 192 | B3.d | —Cl | —F | H | —F | —F | *RS |
| 80 | B1.a | —Cl | —Cl | H | H | H | *RS |
| 81 | B1.c | —Cl | —Cl | H | H | —Cl | *RS |
| 82 | B1.a | —Cl | —Cl | H | H | —CH₃ | *RS |
| 83 | B5 | —Cl | —Cl | H | H | —COOH | *RS |
| 84 | B1.a | —Cl | —Cl | H | H | —OCH₃ | *RS |
| 85 | B1.c | —Cl | —Cl | H | —F | H | *RS |
| 86 | B1.a | —Cl | —CF₃ | H | H | H | *RS |
| 42 | B16 | —Cl | —NH₂ | H | H | H | *RS; •HCl |
| 12 | B1.1 | —Cl | —NO₂ | H | H | H | *RS; •HCl |
| 12a | B1.1 | —Cl | —NO₂ | H | H | H | *RS |
| 87 | B1.j | —Br | H | H | H | H | *RS |
| 10 | B1.j | —Br | H | H | H | H | *RS; •HCl |
| 13 | B2.a/B15 | —Br | H | H | H | H | |
| 14 | B2.b | —Br | H | H | H | H | *R; •HCl |
| 88 | B15 | —Br | H | H | H | H | |
| 9 | B1.i | —Br | H | H | H | H | *RS; •HCl |
| 89 | B1.c | —Br | H | H | H | —Cl | *RS |
| 90 | B1.c | —Br | H | H | H | —CF₃ | *RS |
| 23 | B6 | —Br | H | H | H | —COOH | *RS |
| 33 | B9.b | —Br | H | H | H | 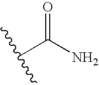 | *RS |
| 32 | B9.a | —Br | H | H | H | 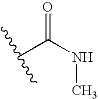 | *RS |
| 91 | B9.d | —Br | H | H | H | 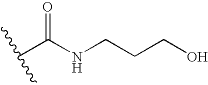 | *RS |
| 37 | B12 | —Br | H | H | H | 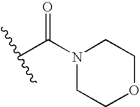 | *RS |
| 34 | B9.c | —Br | H | H | H | 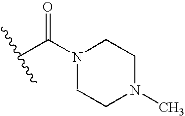 | *RS |
| 39 | B14 | —Br | H | H | H | —OH | *RS |
| 8 | B1.h | —Br | H | H | H | —OCH₃ | *RS |
| 35 | B10 | —Br | H | H | H | —NH₂ | *RS |
| 92 | B1.c | —Br | H | H | —F | H | *RS |
| 93 | B1.h | —Br | H | H | —OCH₃ | H | *RS |
| 193 | B18 | —Br | —H | —H | —COOH | —H | *RS |
| 94 | B1.a | —Br | H | —F | H | H | *RS |
| 95 | B1.c | —Br | H | —F | H | —Cl | *RS |
| 96 | B1.c | —Br | H | —F | H | —CF3 | *RS |
| 97 | B5 | —Br | H | —F | H | —COOH | *RS |
| 98 | B1.c | —Br | H | —F | —F | H | *RS |
| 16a | B3.b | —Br | —F | H | H | H | *R; •HBr |
| 16b | B3.b | —Br | —F | H | H | H | |
| 99 | B5 | —Br | —F | H | H | —COOH | *RS |
| 100 | B5 | —Br | —CH₃ | H | H | —COOH | *RS |
| 101 | B1.j | —CH₃ | H | H | H | —CH₃ | *RS; •HCl |
| 102 | B1.a | —CF₃ | H | H | H | H | *RS |
| 103 | B1.c | —CF₃ | H | H | H | —Cl | *RS |
| 104 | B1.a | —CF₃ | H | H | H | —CH₃ | *RS |
| 105 | B1.a | —CF₃ | H | H | H | —OCH₃ | *RS |
| 106 | B1.c | —CF₃ | H | H | —F | H | *RS |
| 36 | B11 | —COOCH₃ | H | H | H | H | *RS; •HCl |
| 107 | B1.c | —OCH₃ | H | H | H | H | *RS; •HCl |
| 108 | B8.f | —OCH₃ | H | —OCH₃ | H | H | *RS |
| 109 | B1.a | —OCF₂H | H | H | H | H | *RS |
| 110 | B1.c | —OCF₂H | H | H | H | —CF₃ | *RS |
| 111 | B1.c | —OCF₂H | H | H | —F | H | *RS |
| 112 | B1.a | —OCF₃ | H | H | H | H | *RS |
| 113 | B1.c | —OCF₃ | H | H | —F | H | *RS |
| 114 | B1.c | —OCF₃ | H | H | H | —Cl | *RS |
| 115 | B1.c | —OCF₃ | H | H | H | —CF₃ | *RS |
| 116 | B1.a | —SCH₃ | H | H | H | H | *RS |
| 117 | B1.c | —SCH₃ | H | H | H | —Cl | *RS |
| 118 | B1.c | —SCH₃ | H | H | —F | H | *RS |
| 119 | B1.a | 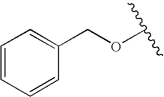 | H | H | H | H | *RS |
| 186 | B17 | H | H | NO₂ | NO₂ | H | *RS |

TABLE 2

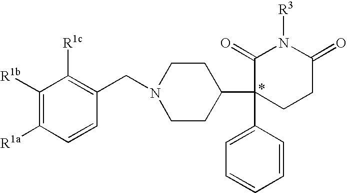

| | Exp. | | | | | Stereochem./ |
|---|---|---|---|---|---|---|
| Co. No. | No. | R^{1a} | R^{1b} | R^{1c} | R³ | Salt |
| 120 | B1.a | —Cl | H | H | —CH₃ | *RS |
| 121 | B1.a | —Cl | H | H | 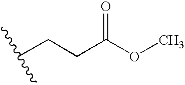 | *RS |
| 21 | B4.a | —Cl | H | H | 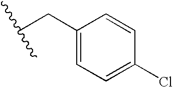 | *S; •HCl |
| 21a | B4.a | —Cl | H | H | 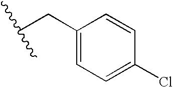 | *S |
| 189 | B4b | —Cl | H | H | 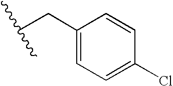 | *R; •HCl |
| 189a | B4b | —Cl | H | H | 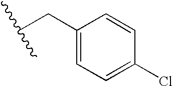 | *R |
| 122 | B1.a | —Cl | —Cl | H | —CH₃ | *RS |
| 123 | B1.a | —Cl | H | —Cl | —CH₃ | *RS |
| 124 | B1.a | —Cl | H | —Cl | 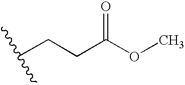 | *RS |
| 125 | B1.a | —Br | H | H | —CH₃ | *RS |
| 126 | B13 | —Br | H | H | —CH₂CH₃ | *RS |
| 127 | B13 | —Br | H | H | 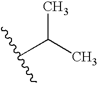 | *RS |
| 128 | B13 | —Br | H | H | 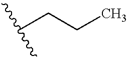 | *RS |
| 129 | B13 | —Br | H | H | 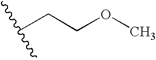 | *RS |
| 130 | B1.a | —Br | H | H | 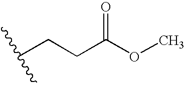 | *RS |
| 38 | B13 | —Br | H | H | 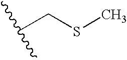 | *RS |
| 38a | B13 | —Br | H | H | 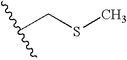 | *RS; •HCl |
| 131 | B1.a | —CF₃ | H | H | —CH₃ | *RS |
| 132 | B1.a | —CF₃ | H | H | 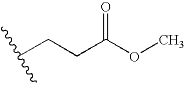 | *RS |

TABLE 3

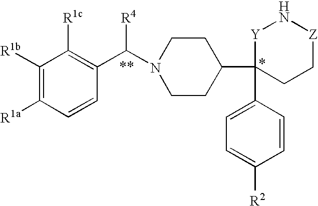

| | Exp. | | | | | | | | Stereochem./ |
|---|---|---|---|---|---|---|---|---|---|
| Co. No. | No. | R^{1a} | R^{1b} | R^{1c} | R² | R⁴ | Y | Z | Salt |
| 133 | B1.e | —F | H | H | H | H | —CH₂— | 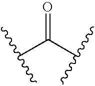 | *RS |
| 134 | B3.a | —F | —Cl | H | —F | H | —CH₂— | 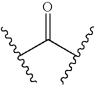 | *RS |
| 135 | B1.e | —Cl | H | H | H | H | —CH₂— | 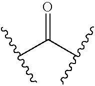 | *RS |
| 136 | B3.a | —Cl | H | H | —F | H | —CH₂— | 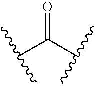 | *RS |
| 137 | B1.e | —Cl | H | —Cl | H | H | —CH₂— | 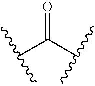 | *RS |
| 138 | B3.a | —Cl | H | —Cl | —F | H | —CH₂— | 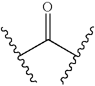 | *RS |
| 139 | B3.a | —Cl | —F | H | —F | H | —CH₂— | 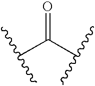 | *RS |
| 140 | B1.e | —Cl | —Cl | H | H | H | —CH₂— | 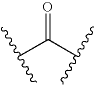 | *RS |
| 141 | B3.a | —Cl | —Cl | H | —F | H | —CH₂— | 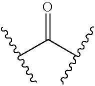 | *RS |
| 5 | B1.e | —Br | H | H | H | H | —CH₂— | 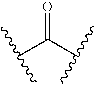 | *RS |
| 142 | B1.k | —Br | H | H | H | H | 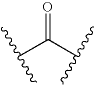 | —CH₂— | *RS |
| 11 | B1.k | —Br | H | H | H | —CH₃ | 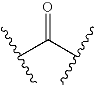 | —CH₂— | *RS; **RS |
| 143 | B3.a | —Br | H | H | —F | H | —CH₂— | 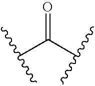 | *RS |
| 144 | B3.a | —Br | H | H | —F | —CH₃ | —CH₂— | 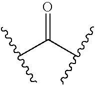 | *RS; **RS |
| 15 | B3.a | —Br | H | —F | —F | H | —CH₂— | 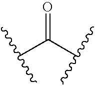 | *RS |
| 145 | B3.a | —Br | —CH₃ | H | —F | H | —CH₂— | 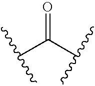 | *RS |
| 146 | B1.e | —CF₃ | H | H | H | H | —CH₂— | 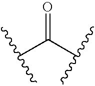 | *RS |
| 147 | B3.a | —OCF₂H | H | H | —F | H | —CH₂— | 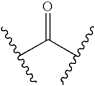 | *RS |
| 148 | B1.e | 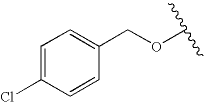 | H | H | H | H | —CH₂— | 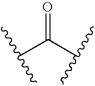 | *RS |

TABLE 4

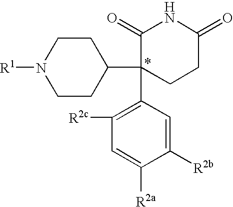

| Co. | Exp. | | | | | Stereochem./ |
|---|---|---|---|---|---|---|
| No. | No. | R¹ | R^{2a} | R^{2b} | R^{2c} | Salt |
| 28 | B8.c | 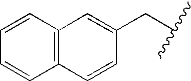 | H | H | H | *RS |
| 149 | B8.a | 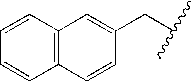 | H | —Cl | H | *RS |
| 150 | B8.a | 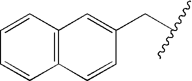 | —F | H | H | *RS |
| 151 | B1.a | 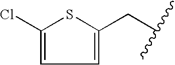 | H | H | H | *RS |
| 152 | B1.c | 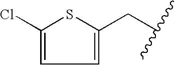 | H | —Cl | H | *RS |
| 153 | B1.c | 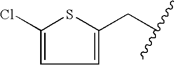 | —F | H | H | *RS |
| 154 | B8.f | 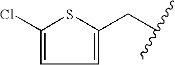 | H | H | H | *RS |
| 24 | B7 | 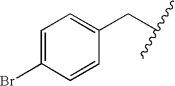 | H | H | 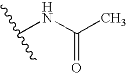 | *RS |
| 6 | B1.f | 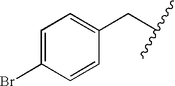 | H | —SO₃H | —OCH₃ | *RS |
| 194 | B19 | 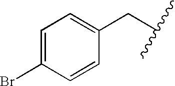 | —F | —H | —F | *RS |
| 195 | B20.c | 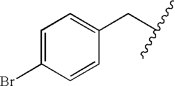 | —F | —H | —F | *S, •HCl |
| 166 | B20.c | 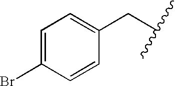 | —F | —H | —F | *R, •HCl |
| 25 | B7 | 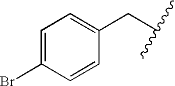 | 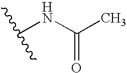 | H | H | *RS |
| 196 | B19 | 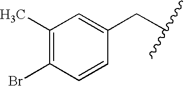 | —F | —H | —F | *RS |
| 155 | B8.f | 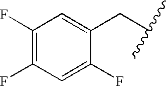 | H | H | H | *RS |
| 156 | B8.a | 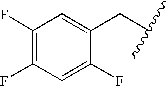 | —F | H | H | *RS |
| 197 | B19 | 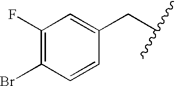 | —F | —H | —F | *RS |
| 198 | B19 | 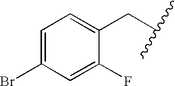 | —F | —H | —F | *RS |
| 199 | B19 | 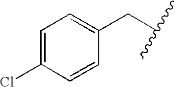 | —F | —H | —F | *RS |
| 200 | B19 | 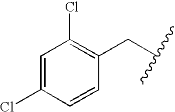 | —F | —H | —F | *RS |
| 201 | B19 | 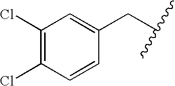 | —F | —H | —F | *RS |
| 202 | B19 | 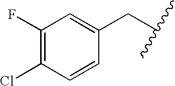 | —F | —H | —F | *RS |
| 203 | B20.a | 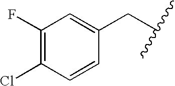 | —F | —H | —F | *R, •HCl |
| 204 | B20.b | 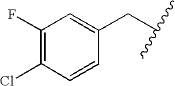 | —F | —H | —F | *S, •HCl |
| 157 | B8.f | 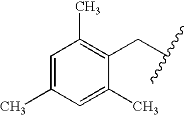 | H | H | H | *RS |
| 205 | B19 | 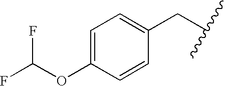 | —F | —H | —F | *RS |
| 158 | B1.a | 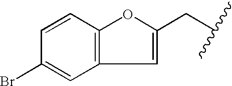 | H | H | H | *RS |
| 159 | B1.c | 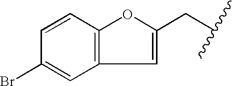 | H | —Cl | H | *RS |
| 160 | B5 | 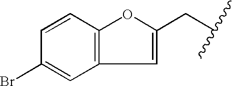 | H | —COOH | H | *RS |
| 161 | B1.c | 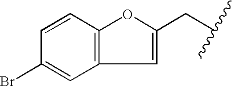 | —F | H | H | *RS |
| 206 | B19 | 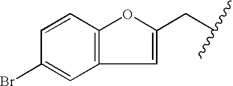 | —F | —H | —F | *RS |
| 162 | B1.c | 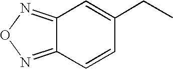 | H | —Cl | H | *RS |
| 163 | B1.c | 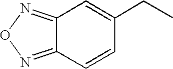 | —F | H | H | *RS |
| 207 | B19 | 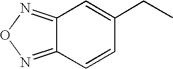 | —F | —H | —F | *RS |
| 1 | B1.a | 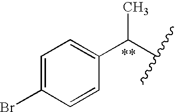 | H | H | H | *RS; **RS |
| 18 | B3.c | 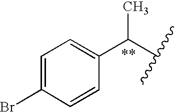 | H | H | H | *R; **R or S |
| 17 | B3.c | 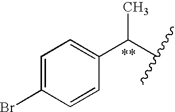 | H | H | H | *R; **S or R |
| 164 | B1.c | 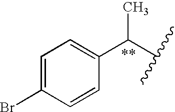 | H | —Cl | H | *RS; **RS |
| 165 | B5 | 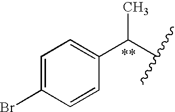 | H | —COOH | H | *RS, **RS |
| 3 | B1.c | 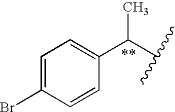 | —F | H | H | *RS, **RS |
| 208 | B19 | 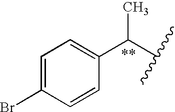 | —F | —H | —F | *RS, **RS |

TABLE 5

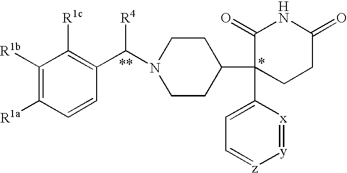

| Co. | Exp. | | | | | | | | Stereochem./ |
|---|---|---|---|---|---|---|---|---|---|
| No. | No. | x | y | z | R^{1a} | R^{1b} | R^{1c} | R⁴ | Salt |
| 167 | B1.b | C | C | N | —F | —Cl | H | H | *RS |
| 168 | B1.b | C | C | N | —Cl | H | —Cl | H | *RS |
| 169 | B1.b | C | C | N | —Cl | —Cl | H | H | *RS |
| 170 | B1.b | C | C | N | —Cl | —CF₃ | H | H | *RS |
| 2 | B1.b | C | C | N | —Br | H | H | H | *RS |
| 171 | B1.b | C | C | N | —Br | H | H | —CH₃ | *RS; **RS |
| 172 | B1.b | C | C | N | —Br | H | —F | H | *RS |
| 173 | B1.b | C | C | N | —OCF₃ | H | H | H | *RS |
| 174 | B1.b | C | C | N | 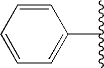 | H | H | H | *RS |
| 175 | B8.a | C | N | C | —F | —F | —F | H | *RS |
| 4 | B1.d | C | N | C | —Cl | H | H | H | *RS |
| 176 | B8.a | C | N | C | —Cl | —F | H | H | *RS |
| 177 | B1.d | C | N | C | —Cl | —Cl | H | H | *RS |
| 178 | B1.d | C | N | C | —Br | H | H | H | *RS |
| 179 | B1.d | C | N | C | —Br | H | —F | H | *RS |
| 180 | B1.d | C | N | C | —OCF₃ | H | H | H | *RS |
| 29 | B8.d | N | C | C | —Cl | —F | H | H | *RS |
| 30 | B8.e | N | C | C | —Br | H | H | H | *RS |

TABLE 6

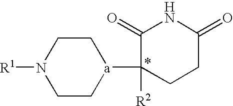

| | | | | | Stereo- |
|---|---|---|---|---|---|
| Co. | Exp. | | | | chem./ |
| No. | No. | a | R¹ | R² | Salt |
| 181 | B8.a | C | 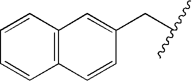 | 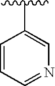 | *RS |
| 182 | B3.a | C | 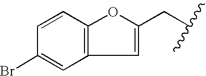 | 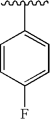 | *RS |
| 183 | B1.b | C | 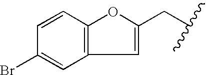 | 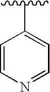 | *RS |
| 184 | B1.d | C | 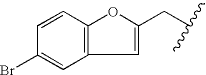 | 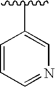 | *RS |
| 7 | B1.g | N | 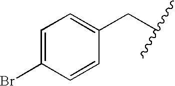 | 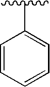 | *RS |
| 27 | B8.b | N | 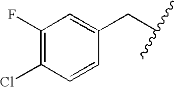 | 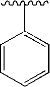 | *RS |

TABLE 7

| Co. | Exp. | |
|---|---|---|
| No. | No. | Structure |
| 209 | B21 | 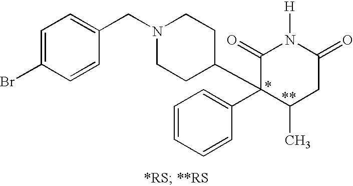 |
| 210 | B22 | 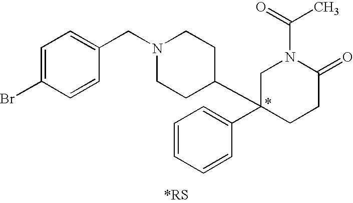 |

### LCMS Conditions

### General Procedure A

The HPLC gradient was supplied by an Alliance HT 2790 (Waters) system comprising a quaternary pump with degasser, an autosampler, a column oven (set at 40° C.) and DAD detector.

Flow from the column was split to a MS detector. The MS detector was configured with an electrospray ionization source. Mass spectra were acquired by scanning from 100 to 1000 in 1 second using a dwell time of 0.1 second. The capillary needle voltage was 3 kV and the source temperature was maintained at 140° C. Nitrogen was used as the nebulizer gas. Data acquisition was performed with a Waters-Micromass MassLynx-Openlynx data system.

### General Procedure B

The LC gradient was supplied by an Acquity UPLC (Waters) system comprising a binary pump, a sample organizer, a column heater (set at 55° C.) and diode-array detector (DAD). Flow from the column was split to a MS detector. The MS detector was configured with an electrospray ionization source. Mass spectra were acquired by scanning from 100 to 1000 in 0.18 seconds using a dwell time of 0.02 seconds. The capillary needle voltage was 3.5 kV and the source temperature was maintained at 140° C. Nitrogen was used as the nebulizer gas. Data acquisition was performed with a Waters-Micromass MassLynx-Openlynx data system.

### Procedure 1

In addition to the general procedure A: Reversed phase HPLC was carried out on a Chromolith (4.6×25 mm) with a flow rate of 3 ml/min. Three mobile phases (mobile phase A: 95% 25 mM ammoniumacetate+5% acetonitrile; mobile phase B: acetonitrile; mobile phase C: methanol) were employed to run a gradient condition from 96% A, 2% B and 2% C, to 49% B and 49% C in 0.9 minutes, to 100% B in 0.3 minutes and hold for 0.2 minutes. An injection volume of 2 μl was used. Cone voltage was 10 V for positive ionization mode and 20 V for negative ionization mode.

### Procedure 2

In addition to the general procedure A: Reversed phase HPLC was carried out on an Xterra MS C18 column (3.5 μm, 4.6×100 mm) with a flow rate of 1.6 ml/min. Three mobile phases (mobile phase A: 95% 25 mM ammoniumacetate+5% acetonitrile; mobile phase B: acetonitrile; mobile phase C: methanol) were employed to run a gradient condition from 100% A to 50% B and 50% C in 6.5 minutes, to 100% B in 1 minute, 100% B for 1 minute and reequilibrate with 100% A for 1.5 minutes. An injection volume of 10 μl was used.

Cone voltage was 10 V for positive ionization mode and 20 V for negative ionization mode.

### Procedure 3

In addition to the general procedure A: Reversed phase HPLC was carried out on a Chromolith (4.6×25 mm) with a flow rate of 3 ml/min. Three mobile phases (mobile phase A: 95% 25 mM ammoniumacetate+5% acetonitrile; mobile phase B: acetonitrile; mobile phase C: methanol) were employed to run a gradient condition from 100% A, to 50% B and 50% C in 0.9 minutes, to 100% B in 0.3 minutes and hold for 0.2 minutes. An injection volume of 2 μl was used. Cone voltage was 10 V for positive ionization mode and 20 V for negative ionization mode.

### Procedure 4

In addition to the general procedure A: Reversed phase HPLC was carried out on an Xterra MS C18 column (3.5 μm, 4.6×100 mm) with a flow rate of 1.6 ml/min. Three mobile phases (mobile phase A: 95% 25 mM ammoniumacetate+5% acetonitrile; mobile phase B: acetonitrile; mobile phase C: methanol) were employed to run a gradient condition from 100% A to 30% A, 35% B; 35% C in 3 minutes to 50% B and 50% C in 3.5 minutes, to 100% B in 0.5 minutes. An injection volume of 10 μl was used. Cone voltage was 10 V for positive ionization mode.

### Procedure 5

In addition to the general procedure A: Reversed phase HPLC was carried out on an Xterra MS C18 column (3.5 μm, 4.6×100 mm) with a flow rate of 1.6 ml/min. Two mobile phases (mobile phase A: 70% methanol+30% H₂O; mobile phase B: 0.1% formic acid in H₂O/methanol 95/5) were employed to run a gradient condition from 100% B to 5% B+95% A in 12 minutes. An injection volume of 10 μl was used. Cone voltage was 10 V for positive ionization mode and 20 V for negative ionization mode.

### Procedure 6

In addition to the general procedure A: Reversed phase HPLC was carried out on an Xterra MS C18 column (3.5 μm, 4.6×100 mm) with a flow rate of 1.6 ml/min. Three mobile phases (mobile phase A: 95% 25 mM ammoniumacetate+5% acetonitrile; mobile phase B: acetonitrile; mobile phase C: methanol) were employed to run a gradient condition from 100% A to 1% A, 49% B and 50% C in 6.5 minutes, to 1% A and 99% B in 1 minute and hold these conditions for 1 minute and reequilibrate with 100% A for 1.5 minutes. An injection volume of 10 μl was used. Cone voltage was 10 V for positive ionization mode and 20 V for negative ionization mode.

### Procedure 7

In addition to general procedure B: Reversed phase UPLC was carried out on a bridged ethylsiloxane/silica (BEH) C18 column (1.7 μm, 2.1×50 mm) with a flow rate of 0.8 ml/min. Two mobile phases (mobile phase A: 0.1% formic acid in H₂O/methanol 95/5; mobile phase B: methanol) were used to run a gradient condition from 95% A to 5% A, 95% B in 1.3 minutes and hold for 0.2 minutes. An injection volume of 0.5 μl was used. Cone voltage was 10 V for positive ionization mode and 20 V for negative ionization mode.

### Optical Rotation

Optical rotations (OR) were performed on a polarimeter (Perkin Elmer). The wavelength, the concentration, the solvent and the temperature for the measurements are indicated after the OR value.

### Melting Points

For a number of compounds, melting points were obtained with a Büchi melting point apparatus B-545 (in open capillary tubes). The heating medium was a metal block. The melting of the sample was visually observed by a magnifying lense and a big light contrast. Melting points were measured with a temperature gradient of either 3 or 10° C./minute. Maximum temperature was 300° C.

TABLE 8
Analytical data (R(t) means retention time in minutes; MH+ means
the protonated mass of the compound; procedure refers to the method
used for LCMS). (When a compound is a mixture of isomers which give
different peaks in the LCMS method, only the retention time of the main
component is given in the LCMS table)

| Comp. | LCMS | | | melting point/optical |
|---|---|---|---|---|
| Nr. | R(t) | MH(+) | Procedure | rotation/comment |
| 9 | 6.39 | 455 | 6 | Measured as the free base |
| 15 | 0.87 | 463 | 7 | |
| 43 | 0.81 | 381 | 7 | Measured as the free base |
| 45 | 0.81 | 381 | 7 | Measured as the free base |
| 101 | 5.29 | 377 | 6 | Measured as the free base |
| 107 | 0.82 | 393 | 7 | Measured as the free base |
| 134 | 0.86 | 419 | 7 | |
| 136 | 0.85 | 401 | 7 | |
| 138 | 0.92 | 435 | 7 | |
| 139 | 0.87 | 419 | 7 | |
| 143 | 0.87 | 445 | 7 | |
| 144 | 0.90 | 459 | 7 | |
| 145 | 0.95 | 459 | 7 | |
| 182 | 0.98 | 485 | 7 | |
| 147 | 0.81 | 433 | 7 | |
| 1 | 5.74 | 455 | 2 | |
| 2 | 0.89 | 442 | 1 | |
| 3 | 7.21 | 473 | 4 | |
| 4 | 4.35 | 398 | 2 | |
| 5 | 7.07 | 427 | 4 | |
| 6 | 0.71 | 551 | 1 | |
| 7 | 7.55 | 442 | 4 | |
| 8 | 6.32 | 471 | 6 | |
| 10 | 1.03 | 441 | 3 | Measured as the free base |
| 11 | 5.61 | 441 | 2 | |
| 12 | 6.31 | 442 | 6 | Measured as the free base |
| | | | | m.p.: 244.4-248.3° C. |
| 13 | 6.3 | 441 | 6 | m.p.: 179-180° C. |
| 14 | 6.31 | 441 | 6 | Measured as the free base |
| 16 | 5.66 | 459 | 2 | Measured as the free base |
| 17 | 6.38 | 455 | 6 | |
| 18 | 6.43 | 455 | 6 | |
| 19 | | | | OR: +82.95° (589 nm, c 0.129 |
| | | | | w/v % MeOH, 20° C.) |
| 20 | | | | OR: −104.96° (589 nm, c 0.141 |
| | | | | w/v % MeOH, 20° C.) |
| 21 | 1.2 | 521 | 7 | Measured as the free base |
| 22 | 4.36 | 459 | 6 | |
| 23 | 0.7 | 486 | 1 | |
| 24 | 4.99 | 498 | 6 | |
| 25 | 4.84 | 498 | 2 | |
| 26 | 7.09 | 433 | 4 | |
| 27 | 5.84 | 416 | 2 | |
| 28 | 5.49 | 413 | 2 | |
| 29 | 5.07 | 416 | 5 | |
| 30 | 2.04 | 442 | 5 | |
| 31 | 7.1 | 415 | 4 | |
| 32 | 4.39 | 498 | 6 | |
| 33 | 3.63 | 484 | 6 | |
| 34 | 2.73 | 567 | 5 | |
| 35 | 3.31 | 456 | 5 | |
| 36 | 0.98 | 421 | 1 | Measured as the free base |
| 37 | 6.41 | 554 | 4 | |
| 38 | 8.11 | 501 | 4 | |
| 39 | 0.98 | 457 | 1 | |
| 40 | 5.82 | 415 | 2 | m.p.: 192-194.3° C. |
| 41 | 5.81 | 415 | 2 | m.p.: 192.3-196.3° C. |
| 42 | 5.18 | 412 | 6 | m.p.: 287.8° C. |
| | | | | LCMS measured as the free base |
| 44 | 6.78 | 381 | 4 | |
| 46 | 5.71 | 411 | 4 | |
| 47 | 1.03 | 399 | 1 | |
| 48 | 7.16 | 433 | 4 | |
| 49 | 6.88 | 417 | 4 | |
| 50 | 6.68 | 417 | 4 | |
| 51 | 7.47 | 451 | 4 | |
| 52 | 7.61 | 431 | 4 | |
| 53 | 7.05 | 447 | 4 | |
| 54 | 7.36 | 435 | 4 | |
| 55 | 1.09 | 415 | 1 | |
| 56 | 7.17 | 433 | 4 | |
| 57 | 6.63 | 459 | 4 | |
| 58 | 7.41 | 477 | 4 | |
| 59 | 6.64 | 395 | 4 | |
| 60 | 6.18 | 411 | 4 | |
| 61 | 7.23 | 397 | 4 | |
| 63 | 7.22 | 431 | 4 | |
| 64 | 7.39 | 411 | 4 | |
| 65 | 7.3 | 465 | 4 | |
| 66 | 3.93 | 441 | 6 | |
| 67 | 5.46 | 427 | 2 | |
| 68 | 6.92 | 415 | 4 | |
| 69 | 4.21 | 427 | 2 | |
| 70 | 8.01 | 431 | 4 | |
| 71 | 8.21 | 465 | 4 | |
| 72 | 8.16 | 445 | 4 | |
| 73 | 5.05 | 475 | 6 | |
| 74 | 7.81 | 461 | 4 | |
| 75 | 7.86 | 449 | 4 | |
| 76 | 5.91 | 415 | 2 | Measured as the free base |
| 77 | 7.35 | 445 | 4 | |
| 78 | 7.39 | 429 | 4 | |
| 79 | 7.69 | 449 | 4 | |
| 80 | 7.66 | 431 | 4 | |
| 81 | 7.87 | 465 | 4 | |
| 82 | 8.02 | 445 | 4 | |
| 83 | 4.4 | 475 | 6 | |
| 84 | 7.71 | 461 | 4 | |
| 85 | 7.52 | 449 | 4 | |
| 86 | 1.15 | 465 | 1 | |
| 87 | 1.03 | 441 | 3 | |
| 88 | 5.63 | 441 | 2 | OR: +99.46° (589 nm, c 0.4112 |
| | | | | w/v %, MeOH, 20° C.) |
| 89 | 7.53 | 475 | 4 | |
| 90 | 7.5 | 509 | 4 | |
| 91 | 5.41 | 542 | 6 | |
| 92 | 7.14 | 459 | 4 | |
| 93 | 5.57 | 471 | 2 | |
| 94 | 5.97 | 459 | 2 | |
| 95 | 7.73 | 493 | 4 | |
| 96 | 7.67 | 527 | 4 | |
| 97 | 4.49 | 503 | 6 | |
| 98 | 7.38 | 477 | 4 | |
| 99 | 4.49 | 503 | 6 | |
| 100 | 4.42 | 499 | 6 | |
| 102 | 7.34 | 431 | 4 | |
| 103 | 7.46 | 465 | 4 | |
| 104 | 7.66 | 445 | 4 | |
| 105 | 7.37 | 461 | 4 | |
| 106 | 7.14 | 449 | 4 | |
| 108 | 5.51 | 423 | 4 | |
| 109 | 4.99 | 429 | 2 | |
| 110 | 6.85 | 497 | 4 | |
| 111 | 6.48 | 447 | 4 | |
| 112 | 1.11 | 447 | 1 | |
| 113 | 7.16 | 465 | 4 | |
| 114 | 7.48 | 481 | 4 | |
| 115 | 7.57 | 515 | 4 | |
| 116 | 0.99 | 409 | 1 | |
| 117 | 6.9 | 443 | 4 | |
| 118 | 6.53 | 427 | 4 | |
| 119 | 7.38 | 469 | 4 | |
| 120 | 7.82 | 411 | 4 | |
| 121 | 5.02 | 483 | 2 | |
| 122 | 8.22 | 445 | 4 | |
| 123 | 8.39 | 445 | 4 | |
| 124 | 5.6 | 517 | 2 | |
| 125 | 7.99 | 456 | 4 | m.p.: 298.1-303.1° C. |
| 126 | 8.01 | 469 | 4 | |
| 127 | 8.49 | 483 | 4 | |
| 128 | 8.28 | 483 | 4 | |
| 129 | 6.2 | 499 | 2 | |
| 130 | 5.13 | 527 | 2 | |
| 131 | 7.67 | 445 | 4 | |
| 132 | 5.22 | 517 | 2 | |
| 133 | 5.95 | 367 | 4 | |
| 135 | 6.78 | 383 | 4 | |
| 137 | 7.78 | 417 | 4 | |
| 140 | 7.52 | 417 | 4 | |
| 141 | 6.61 | 435 | 6 | |
| 142 | 5.54 | 427 | 2 | |
| 146 | 6.95 | 417 | 4 | |
| 148 | 7.03 | 489 | 4 | |
| 149 | 7.53 | 447 | 4 | |
| 150 | 7.31 | 431 | 4 | |
| 151 | 1.1 | 403 | 1 | |
| 152 | 7.48 | 437 | 4 | |
| 153 | 7.16 | 421 | 4 | |
| 154 | 7.22 | 447 | 4 | |
| 155 | 6.98 | 417 | 4 | |
| 156 | 7.06 | 435 | 4 | |
| 157 | 7.68 | 405 | 4 | |
| 158 | 6.07 | 481 | 2 | |
| 159 | 7.8 | 515 | 4 | |
| 160 | 5.12 | 525 | 6 | |
| 161 | 7.5 | 499 | 4 | |
| 162 | 6.93 | 439 | 4 | |
| 163 | 6.68 | 423 | 4 | |
| 164 | 7.6 | 489 | 4 | |
| 165 | 4.18 | 499 | 6 | |
| 167 | 5.64 | 416 | 4 | |
| 168 | 6.55 | 432 | 4 | |
| 169 | 6.66 | 432 | 4 | |
| 170 | 6.84 | 466 | 4 | |
| 171 | 6.18 | 456 | 4 | |
| 172 | 6.43 | 460 | 4 | |
| 173 | 6.2 | 448 | 4 | |
| 174 | 5.76 | 440 | 4 | |
| 175 | 5.99 | 418 | 4 | |
| 176 | 6.23 | 416 | 4 | |
| 177 | 5.41 | 432 | 2 | |
| 178 | 4.81 | 442 | 2 | |
| 179 | 6.43 | 460 | 4 | |
| 180 | 0.95 | 448 | 1 | |
| 181 | 5.73 | 414 | 4 | |
| 183 | 6.46 | 482 | 4 | |
| 184 | 6.5 | 482 | 4 | |
| 186 | 5.05 | 453 | 2 | |
| 189 | 7.45 | 521 | 6 | Measured as the free base |
| 190 | 4.16 | 459 | 6 | |
| 191 | 3.76 | 441 | 6 | |
| 192 | 6.58 | 451 | 6 | |
| 193 | 3.95 | 485 | 6 | |
| 194 | 6.52 | 477 | 6 | |
| 195 | 6.79 | 477 | 5 | m.p.: 273.9-278.3° C. |
| | | | | LCMS measured as the free base |
| 196 | 6.81 | 491 | 6 | |
| 197 | 6.71 | 495 | 6 | |
| 198 | 6.79 | 495 | 6 | |
| 199 | 6.41 | 433 | 6 | |
| 200 | 7.15 | 467 | 6 | |
| 201 | 4.94 | 467 | 6 | |
| 202 | 6.65 | 451 | 6 | |
| 203 | 6.79 | 451 | 5 | m.p.: 282.0-285.9° C. |
| | | | | LCMS measured as the free base |
| 204 | 6.57 | 451 | 6 | m.p.: 282.0-283.8° C. |
| | | | | LCMS measured as the free base |
| 205 | 5.95 | 465 | 6 | |
| 206 | 6.84 | 517 | 6 | |
| 207 | 6.24 | 441 | 6 | |
| 208 | 6.66 | 491 | 6 | |
| 209 | 5.79 | 455 | 5 | |
| 210 | 6.72 | 469 | 6 | |

### D. Pharmacological Example

### CXCR3 Receptor Inhibition was Examined in a [³⁵S]GTPγS Exchange Assay

The exchange of guanosine 5′-[³⁵S]triphosphate was measured on membranes of human CXCR3-transfected CHO cells. [³⁵S]GTPγS exchange assays were performed in 96-well plates with 10 μg of membrane protein/well using basic flashplates (Perkin Elmer). Compounds were dissolved in DMSO and diluted with incubation buffer to yield required concentrations with 9% DMSO. Incubation buffer is composed of 20 mM HEPES, 100 mM NaCl, 3 μM GDP and 1 mM MgCl₂, pH 7.4. Membrane incubation buffer is incubation buffer supplemented with 14.3 μg/ml saponin. Compound, membranes, hI-TAC (interferon-inducible T-cell alpha chemoattractant) and [³⁵S]GTPγS were added in a total volume of 200 μl. First, 20 μl of the appropriate compound dilution and 140 μl membranes from CXCR3-CHO cells were dissolved in membrane incubation buffer and pre-incubated for 30 minutes at 30° C. Then, 20 μl of hI-TAC dissolved at 30 nM in incubation buffer was added to the membranes and the mixture containing 1% of DMSO was incubated for another 30 minutes at 30° C. Finally, 20 μl [³⁵S]GTPγS (˜119 Ci/mmol, Amersham) dissolved at 2.5 nM in incubation buffer was added. After 1 minute shaking and 30 minutes incubation at 30° C., flashplates were centrifuged for 5 minutes at 2500 rpm at room temperature. Flashplate bound radioactivity was determined by liquid scintillation counting. Basal GTPγS-binding was measured in 8 wells with membranes incubated in the same volume with 1% DMSO, without I-TAC. Maximal GTPγS-binding was measured in 8 wells with membranes incubated with 1% DMSO and 3 nM I-TAC. The IC₅₀ value is calculated as the molar concentration of the test compound, which inhibits 50% of specific I-TAC-induced GTPγS-binding. IC₅₀ values were calculated using non-linear regression in Graphpad Prism.

Table 9 reports pIC₅₀ values obtained in the above-described test for compounds of formula (I). pIC₅₀ defines −log IC₅₀ wherein IC₅₀ is the molar concentration of the test compound which inhibits 50% of specific I-TAC-induced GTPγS-binding.

TABLE 9

| Comp. Nr. | pIC₅₀ |
|---|---|
| 1 | 6.4 |
| 2 | 6.2 |
| 3 | 6.7 |
| 4 | 6.1 |
| 5 | 6.3 |
| 6 | 6.8 |
| 7 | 6.6 |
| 8 | 5.8 |
| 9 | 5.7 |
| 10 | 6.5 |
| 11 | 6.1 |
| 13 | 6.5 |
| 14 | 6.2 |
| 15 | 6.4 |
| 16a | 6.5 |
| 17 | 5.7 |
| 18 | 6.4 |
| 19 | 5.9 |
| 20 | 6.1 |
| 22 | 6.6 |
| 23 | 6.2 |
| 24 | 6.0 |
| 26 | 6.7 |
| 27 | 5.6 |
| 28 | 5.4 |
| 29 | 6.1 |
| 30 | 6.3 |
| 31 | 6.7 |
| 32 | 5.5 |
| 33 | 5.5 |
| 34 | 5.9 |
| 35 | 6.5 |
| 36 | 5.9 |
| 37 | 6.1 |
| 38 | 6.0 |
| 39 | 6.0 |
| 40 | 6.4 |
| 41 | 6.4 |
| 43 | 5.5 |
| 44 | 5.3 |
| 45 | 5.4 |
| 46 | 5.4 |
| 47 | 5.7 |
| 48 | 5.0 |
| 49 | 5.4 |
| 50 | 5.8 |
| 51 | 5.6 |
| 52 | 5.9 |
| 53 | 5.7 |
| 54 | 5.9 |
| 55 | 5.5 |
| 56 | 5.3 |
| 57 | 5.4 |
| 58 | 5.1 |
| 59 | 5.9 |
| 60 | 5.5 |
| 61 | 6.0 |
| 62 | 6.1 |
| 63 | 5.7 |
| 64 | 5.7 |
| 65 | 5.6 |
| 66 | 6.1 |
| 67 | 6.0 |
| 68 | 6.1 |
| 69 | 5.5 |
| 70 | 5.6 |
| 71 | 5.6 |
| 72 | 5.2 |
| 73 | 6.3 |
| 74 | 5.4 |
| 75 | 5.6 |
| 76 | 6.5 |
| 77 | 6.1 |
| 78 | 6.1 |
| 79 | 6.6 |
| 80 | 5.9 |
| 81 | 5.4 |
| 82 | 5.5 |
| 83 | 6.4 |
| 84 | 5.7 |
| 85 | 6.1 |
| 86 | 5.7 |
| 87 | 6.1 |
| 88 | 5.9 |
| 89 | 5.8 |
| 90 | 5.8 |
| 91 | 5.9 |
| 92 | 6.0 |
| 93 | 5.4 |
| 94 | 6.3 |
| 95 | 5.8 |
| 96 | 5.5 |
| 97 | 6.3 |
| 98 | 6.2 |
| 99 | 6.4 |
| 101 | 5.6 |
| 102 | 5.4 |
| 103 | 5.4 |
| 104 | 5.4 |
| 105 | 5.0 |
| 106 | 5.4 |
| 107 | 5.4 |
| 108 | 5.1 |
| 109 | 6.0 |
| 110 | 5.1 |
| 111 | 6.0 |
| 112 | 6.0 |
| 113 | 5.3 |
| 114 | 5.1 |
| 115 | 5.0 |
| 116 | 5.3 |
| 117 | 5.2 |
| 118 | 5.0 |
| 119 | 5.2 |
| 120 | 5.8 |
| 121 | 5.2 |
| 122 | 5.3 |
| 123 | 5.0 |
| 124 | 6.0 |
| 125 | 5.6 |
| 126 | 5.6 |
| 127 | 5.3 |
| 128 | 5.4 |
| 129 | 5.7 |
| 130 | 5.4 |
| 131 | 5.4 |
| 132 | 6.2 |
| 133 | 5.8 |
| 134 | 5.4 |
| 135 | 6.0 |
| 136 | 6.1 |
| 137 | 6.2 |
| 138 | 5.8 |
| 139 | 5.8 |
| 140 | 6.1 |
| 141 | 5.8 |
| 142 | 5.8 |
| 143 | 6.2 |
| 144 | 6.2 |
| 145 | 6.1 |
| 146 | 5.6 |
| 147 | 5.4 |
| 148 | 5.2 |
| 149 | 5.3 |
| 150 | 6.0 |
| 151 | 5.5 |
| 152 | 6.1 |
| 153 | 5.9 |
| 154 | 5.6 |
| 155 | 5.6 |
| 156 | 6.1 |
| 157 | 5.5 |
| 158 | 5.9 |
| 159 | 5.0 |
| 160 | 6.5 |
| 161 | 5.5 |
| 162 | 5.0 |
| 163 | 5.4 |
| 164 | 6.0 |
| 165 | 6.1 |
| 166 | 7.2 |
| 167 | 5.4 |
| 168 | 5.8 |
| 169 | 6.0 |
| 170 | 5.4 |
| 171 | 5.8 |
| 172 | 5.7 |
| 173 | 5.7 |
| 174 | 5.3 |
| 175 | 6.0 |
| 176 | 5.7 |
| 177 | 6.0 |
| 178 | 6.0 |
| 179 | 5.7 |
| 180 | 5.6 |
| 181 | 5.0 |
| 182 | 5.5 |
| 183 | 5.9 |
| 184 | 5.8 |
| 186 | <5 |
| 189 | 5.7 |
| 190 | 5.2 |
| 191 | 5.3 |
| 192 | 5.6 |
| 193 | 5.3 |
| 194 | 7.0 |
| 195 | 6.5 |
| 196 | 6.5 |
| 197 | 6.4 |
| 198 | 6.6 |
| 199 | 6.6 |
| 200 | 6.2 |
| 201 | 6.4 |
| 202 | 6.6 |
| 203 | 7.1 |
| 204 | 6.6 |
| 205 | 6.2 |
| 206 | 5.9 |
| 207 | 6.4 |
| 208 | 6.8 |
| 209 | 6.3 |
| 210 | 5.9 |

1. A compound of formula 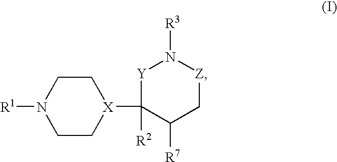
a N-oxide thereof, a pharmaceutically acceptable salt thereof, a stereochemically isomeric form thereof or a solvate thereof, wherein
X represents N or CH;
Y and Z each independently represent C(═O) or CH₂ provided that at least one of Y and Z represents C(═O);
R¹ represents CH(R⁴)-aryl or CH(R⁴)-heteroaryl;
R² represents aryl² or heteroaryl;
R³ represents hydrogen; C₁₋₄alkylcarbonyl; C₁₋₆alkyl optionally substituted with C₁₋₆alkyloxy, C₁₋₆alkylthio, C₁₋₆alkyloxycarbonyl or aryl¹;
R⁴ represents hydrogen or C₁₋₄alkyl;
R⁵ and R⁶ each independently represent hydrogen, or C₁₋₆alkyl optionally substituted with hydroxyl; or
R⁵ and R⁶ together with the nitrogen to which they are attached form a monocyclic heterocycle selected from piperidinyl, piperazinyl, morpholinyl, or thiomorpholinyl, each of said rings optionally substituted with C₁₋₄alkyl;
R⁷ represents hydrogen or C₁₋₄alkyl;
aryl represents unsubstituted naphthyl; or phenyl or naphthyl, each of said phenyl or naphthyl substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di-(C₁₋₄alkyl)amino, C₁₋₄alkylcarbonylamino, aryl¹, aryl¹C₁₋₄alkyloxy, aryl¹oxy, or aryl¹C(═O)—;
aryl¹ represents phenyl or phenyl substituted with 1, 2 or 3 substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, aminocarbonyl, mono- or di(C₁₋₄alkyl)aminocarbonyl, amino, or mono- or di(C₁₋₄alkyl)amino;
aryl² represents phenyl or naphthyl, each of said rings optionally substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—;
heteroaryl represents a monocyclic heterocycle selected from pyrrolinyl, imidazolinyl, pyrazolinyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, piperidinyl, piperazinyl, morpholinyl, thiomorpholinyl; or a bicyclic heterocycle selected from indolyl, indolizinyl, isoindolyl, indolinyl, benzofuranyl, benzothienyl, indazolyl, benzimidazolyl, benzthiazolyl, purinyl, quinolizinyl, quinolinyl, isoquinolinyl, cinnolinyl, phthalazinyl, quinazolinyl, quinoxalinyl, naphthyridinyl, pteridinyl, benzoxadiazolyl, benzoxazolyl, benzthiazolyl, each of said monocyclic or bicyclic heterocycle optionally being substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O), amino, mono- or di(C₁₋₄alkyl)amino or C₁₋₄alkylcarbonylamino;
provided that when Y and Z each represent C(═O), X represents CH, R³ represents hydrogen, R⁴ represents hydrogen, and R² represents unsubstituted pyridyl or phenyl optionally substituted with one halo or with one C₁₋₄alkyloxy or with one or two C₁₋₄alkyl, then aryl in the definition of R¹ is other than phenyl substituted with one halo or with one or two C₁₋₄alkyl; and
provided that when Y and Z each represent C(═O), X represents CH, R³ represents hydrogen, and R² represents unsubstituted pyridyl or phenyl optionally substituted with one halo or with one C₁₋₄alkyloxy or with one or two C₁₋₄alkyl, then heteroaryl in the definition of R¹ is other than unsubstituted thienyl or unsubstituted pyridyl.

2. A compound as claimed in claim 1 wherein
R³ represents hydrogen; C₁₋₆alkyl optionally substituted with C₁₋₆alkyloxy, C₁₋₆alkylthio, C₁₋₆alkyloxycarbonyl or aryl¹;
R⁷ represents hydrogen.

3. A compound according to claim 1 wherein Y represents CH₂.

4. A compound as claimed in claim 1 wherein Z represents CH₂.

5. A compound as claimed in claim 1 wherein both Y and Z represent C(═O).

6. A compound according to claim 1 wherein X represents CH.

7. A compound according to claim 1 wherein X represents N.

8. A compound according to claim 1 wherein R¹ represents CH(R⁴)-aryl.

9. A compound according to claim 8 wherein aryl represents phenyl substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy; C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O), amino, mono- or di(C₁₋₄alkyl)amino, C₁₋₄alkylcarbonylamino; aryl¹; aryl¹C₁₋₄alkyloxy; aryl¹oxy; or aryl¹C(═O).

10. A compound according to claim 9 wherein aryl represents phenyl substituted with one or two halo.

11. A compound according to claim 1 wherein R² represents aryl².

12. A compound according to claim 11 wherein aryl² represents phenyl optionally substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O), amino, mono- or di-(C₁₋₄alkyl)amino, C₁₋₄alkylcarbonylamino, aryl¹, aryl¹C₁₋₄alkyloxy, aryl¹oxy, or aryl¹C(═O).

13. A compound according to claim 12 wherein aryl² represents phenyl substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O), amino, mono- or di(C₁₋₄alkyl)amino, C₁₋₄alkylcarbonylamino, aryl¹, aryl¹C₁₋₄alkyloxy, aryl¹oxy, or aryl¹C(═O).

14. A compound according to claim 1 wherein R² represents heteroaryl.

15. A compound according to claim 1 wherein R³ represents hydrogen.

16. A compound according to claim 1 wherein R⁴ represents hydrogen.

17. A compound according to claim 1 wherein R⁴ represents C₁₋₄alkyl.

18. A compound according to claim 1 wherein the compound is a compound of formula (I-A) 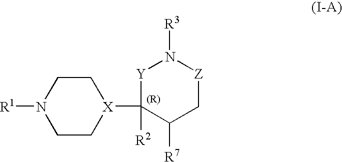
a N-oxide thereof, a pharmaceutically acceptable salt thereof, a stereochemically isomeric form thereof or a solvate thereof.

19. A compound according to claim 1 wherein R³ represents hydrogen;
C₁₋₄alkylcarbonyl; C₁₋₆alkyl optionally substituted with C₁₋₆alkyloxy, C₁₋₆alkylthio, C₁₋₆alkyloxycarbonyl or aryl¹; aryl represents unsubstituted naphthyl; or phenyl substituted with at least one substituent, each substituent independently selected from halo, C₁₋₆alkyl, C₁₋₆alkyloxy, C₁₋₆alkyloxycarbonyl, C₁₋₆alkylthio, polyhalo-C₁₋₆alkyl, polyhaloC₁₋₆alkyloxy, nitro, amino, aryl¹, or aryl¹C₁₋₄alkyloxy; aryl¹ represents phenyl or phenyl substituted with halo; aryl² represents phenyl, optionally substituted with at least one substituent, each substituent independently selected from halo, hydroxyl, C₁₋₆alkyl, C₁₋₆alkyloxy, polyhaloC₁₋₆alkyl, nitro, carboxyl, HO—SO₂—, R⁶R⁵N—C(═O)—, amino, or C₁₋₄alkylcarbonylamino; heteroaryl represents thienyl, pyridyl, benzofuranyl, benzoxadiazolyl, each of said ring systems optionally being substituted with halo; R⁵ and R⁶ each independently represent hydrogen, or C₁₋₆alkyl optionally substituted with hydroxyl; or R⁵ and R⁶ together with the nitrogen to which they are attached form a monocyclic heterocycle selected from piperazinyl or morpholinyl, each of said rings optionally substituted with C₁₋₄alkyl; Y and Z are both C(═O); Y is CH₂ and Z is C(═O); Y is C(═O) and Z is CH₂; X is CH or N.; R⁷ represents hydrogen or methyl.

20. A compound according to claim 1 herein the nitrogen atom carrying the R¹ substituent is not protonated or not quaternized.

21. A compound according to claim 1 wherein the compound is selected from the following compounds

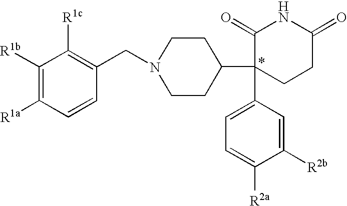

| | | | | | stereo- |
|---|---|---|---|---|---|
| R^{1a} | R^{1b} | R^{1c} | R^{2a} | R^{2b} | chemistry |
| —Br | H | H | H | —COOH | *RS |
| —Cl | —F | H | H | H | *S; (+) |
| —Cl | —F | H | H | H | *R; (−) |
| —Cl | —F | H | H | —COOH | *RS |
| —Cl | —F | H | —F | H | *RS |
| —Br | H | H | H | 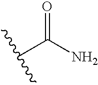 | *RS |
| —Br | H | H | H | —NH₂ | *RS |
| —Br | —F | H | H | H | *R |
| —Br | —F | H | H | —COOH | *RS |
| —Cl | —F | H | H | H | *RS |

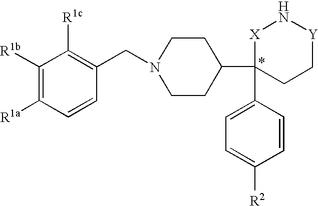
| | | | | | | Stereo- |
|---|---|---|---|---|---|---|
| R^{1a} | R^{1b} | R^{1c} | R² | X | Y | chemistry |
| —Br | H | —F | —F | —CH₂— | 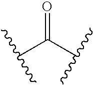 | *RS |

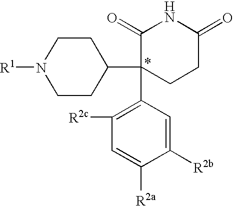
| | | | | stereo- |
|---|---|---|---|---|
| R¹ | R^{2a} | R^{2b} | R^{2c} | chemistry |
| 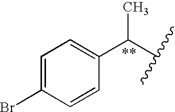 | F | H | F | *RS;**RS |
| 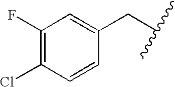 | F | H | F | *RS |
| 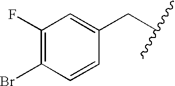 | F | H | F | *RS |
| 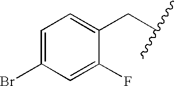 | F | H | F | *RS |
| 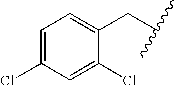 | F | H | F | *RS |
| 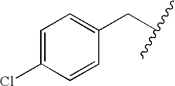 | F | H | F | *RS |
| 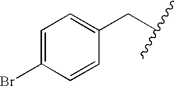 | F | H | F | *RS |
| 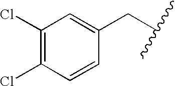 | F | H | F | *RS |
| 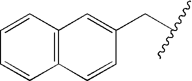 | H | —Cl | H | *RS |
| 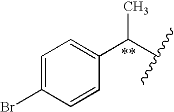 | H | H | H | *R;** R or S |
| 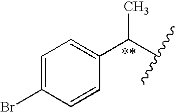 | H | H | H | *R;** S or R |
| 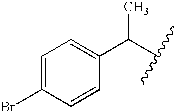 | —F | H | H | *RS;**RS |
| 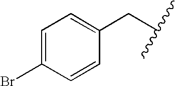 | H | —SO₃H | —OCH₃ | *RS |

a N-oxide thereof, a pharmaceutically acceptable salt thereof, a stereochemically isomeric form thereof or a solvate thereof.

22. A compound according to claim 1 wherein the compound is selected from the following compounds

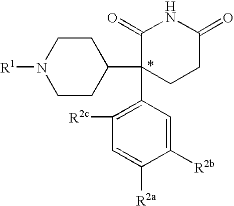

| | | | | stereo- |
|---|---|---|---|---|
| R¹ | R^{2a} | R^{2b} | R^{2c} | chemistry |
| 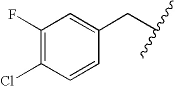 | F | H | F | *RS |
| 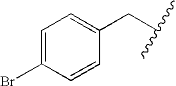 | F | H | F | *RS |
| 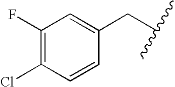 | F | H | F | *R; HCl |
| 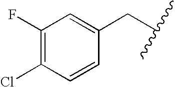 | F | H | F | *S; HCl |
| 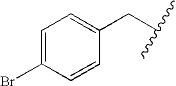 | F | H | F | *R; HCl |
| 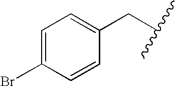 | F | H | F | *S; HCl |

^{a}N-oxide thereof, a pharmaceutically acceptable salt thereof, a stereochemically isomeric form thereof or a solvate thereof.

23. A compound selected from

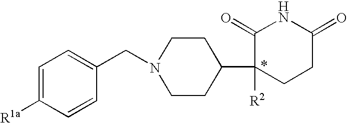

| | | stereo- |
|---|---|---|
| | | chemistry/ |
| R^{1a} | R² | salt |
| —Br | phenyl | *R |
| —Br | phenyl | *R; •HCl |
| —Cl | 3-pyridyl | *RS |

^{a}N-oxide thereof, a pharmaceutically acceptable salt thereof, a stereochemically isomeric form thereof or a solvate thereof.

24. (canceled)

25. A pharmaceutical composition comprising a pharmaceutically acceptable carrier, and as active ingredient a therapeutically effective amount of a compound as claimed in claim 1.

26. A process of preparing a pharmaceutical composition comprising mixing a pharmaceutically acceptable carrier with a therapeutically effective amount of a compound as claimed in claim 1.

27. A method for preventing or treating a disease mediated through activation of the CXCR3 receptor comprising administering to a warm-blooded mammal in need thereof a therapeutically effective amount of compound of formula (I) 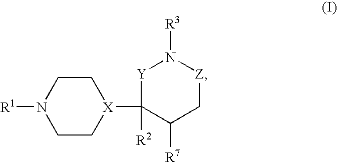
a N-oxide thereof, a pharmaceutically acceptable salt thereof, a stereochemically isomeric form thereof or a solvate thereof, wherein
X represents N or CH;
Y and Z each independently represent C(═O) or CH₂ provided that at least one of Y and Z represents C(═O);
R¹ represents CH(R⁴)-aryl or CH(R⁴)-heteroaryl;
R² represents aryl² or heteroaryl;
R³ represents hydrogen; C₁₋₄alkylcarbonyl; C₁₋₆alkyl optionally substituted with C₁₋₆alkyloxy, C₁₋₆alkylthio, C₁₋₆alkyloxycarbonyl or aryl¹;
R⁴ represents hydrogen or C₁₋₄alkyl;
R⁵ and R⁶ each independently represent hydrogen, or C₁₋₆alkyl optionally substituted with hydroxyl; or
R⁵ and R⁶ together with the nitrogen to which they are attached form a monocyclic heterocycle selected from piperidinyl, piperazinyl, morpholinyl, or thiomorpholinyl, each of said rings optionally substituted with C₁₋₄alkyl;
R⁷ represents hydrogen or C₁₋₄alkyl;
aryl represents unsubstituted naphthyl; or phenyl or naphthyl, each of said phenyl or naphthyl substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—;
aryl¹ represents phenyl or phenyl substituted with 1, 2 or 3 substituents, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, aminocarbonyl, mono- or di(C₁₋₄alkyl)aminocarbonyl, amino, or mono- or di(C₁₋₄alkyl)amino;
aryl² represents phenyl or naphthyl, each of said rings optionally substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O)—, amino, mono- or di(C₁₋₄alkyl)amino, C₁₋₄alkylcarbonylamino, aryl¹, aryl¹C₁₋₄alkyloxy, aryl¹oxy, or aryl¹C(═O)—;
heteroaryl represents a monocyclic heterocycle selected from pyrrolinyl, imidazolinyl, pyrazolinyl, furanyl, thienyl, pyrrolyl, oxazolyl, thiazolyl, imidazolyl, pyrazolyl, isoxazolyl, isothiazolyl, oxadiazolyl, triazolyl, thiadiazolyl, pyridyl, pyridazinyl, pyrimidinyl, pyrazinyl, piperidinyl, piperazinyl, morpholinyl, thiomorpholinyl; or a bicyclic heterocycle selected from indolyl, indolizinyl, isoindolyl, indolinyl, benzofuranyl, benzothienyl, indazolyl, benzimidazolyl, benzthiazolyl, purinyl, quinolizinyl, quinolinyl, isoquinolinyl, cinnolinyl, phthalazinyl, quinazolinyl, quinoxalinyl, naphthyridinyl, pteridinyl, benzoxadiazolyl, benzoxazolyl, benzthiazolyl, each of said monocyclic or bicyclic heterocycle optionally being substituted with at least one substituent, each substituent independently selected from halo, hydroxyl, C₁₋₆alkyl, C₁₋₆alkyloxy, C₁₋₆alkyloxycarbonyl, C₁₋₆alkylcarbonyloxy, C₁₋₆alkylthio, polyhaloC₁₋₆alkyl, polyhaloC₁₋₆alkyloxy, cyano, nitro, carboxyl, HO—SO₂—, C₁₋₄alkyl-SO₂—, R⁶R⁵N—C(═O), amino, mono- or di(C₁₋₄alkyl)amino or ₁₋₄alkylcarbonylamino.

28. The method as claimed in claim 27 wherein the compound is a compound of formula (I-A) 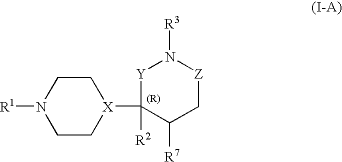
a N-oxide thereof, a pharmaceutically acceptable salt thereof, a stereochemically isomeric form thereof or a solvate thereof

29. The method as claimed in claim 27 wherein the compound is selected from the following compounds

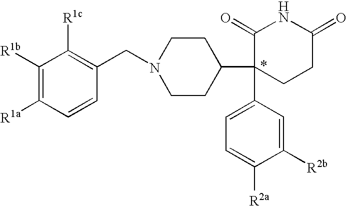

| | | | | | stereo- |
|---|---|---|---|---|---|
| | | | | | chemistry/ |
| R^{1a} | R^{1b} | R^{1c} | R^{2a} | R^{2b} | salt |
| —Br | H | H | H | —COOH | *RS |
| —Cl | —F | H | H | H | *S; (+) |
| —Cl | —F | H | H | H | *R; (−) |
| —Cl | —F | H | H | —COOH | *RS |
| —Cl | —F | H | —F | H | *RS |
| —Br | H | H | H | H | (±) |
| —Br | H | H | H | H | *R |
| —Br | H | H | H | H | *R; •HCl |
| —Br | H | H | H | H | *S |
| —Br | H | H | H | 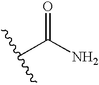 | *RS |
| —Br | H | H | H | —NH₂ | *RS |
| —Br | —F | H | H | H | *R |
| —Br | —F | H | H | —COOH | *RS |
| —Cl | —F | H | H | H | *RS |

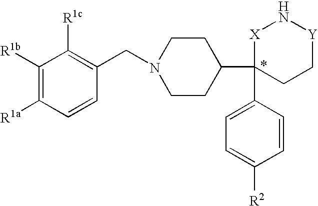
| | | | | | | Stereo- |
|---|---|---|---|---|---|---|
| R^{1a} | R^{1b} | R^{1c} | R² | X | Y | chemistry |
| —Br | H | —F | —F | —CH₂— | 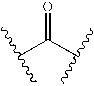 | *RS |

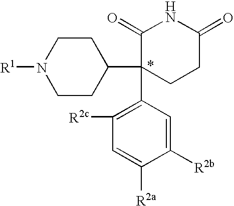
| | | | | stereo- |
|---|---|---|---|---|
| R¹ | R^{2a} | R^{2b} | R^{2c} | chemistry |
| 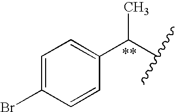 | F | H | F | *RS;**RS |
| 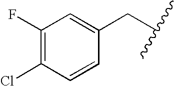 | F | H | F | *RS |
| 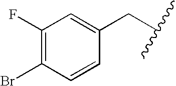 | F | H | F | *RS |
| 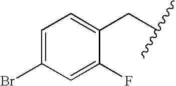 | F | H | F | *RS |
| 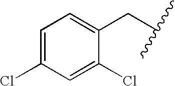 | F | H | F | *RS |
| 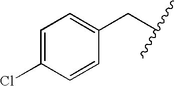 | F | H | F | *RS |
| 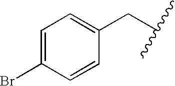 | F | H | F | *RS |
| 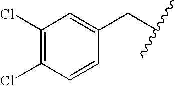 | F | H | F | *RS |
| 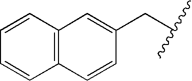 | H | —Cl | H | *RS |
| 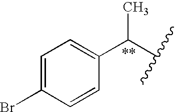 | H | H | H | *R;** R or S |
| 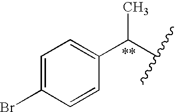 | H | H | H | *R;** S or R |
| 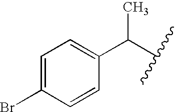 | —F | H | H | *RS |
| 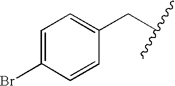 | H | —SO₃H | —OCH₃ | *RS |

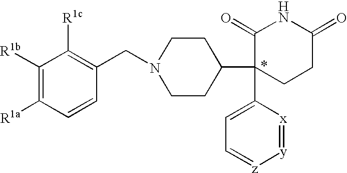
| | | | | | | Stereo- |
|---|---|---|---|---|---|---|
| x | y | z | R^{1a} | R^{1b} | R^{1c} | chemistry |
| C | N | C | —Cl | H | H | *RS |

a N-oxide thereof a pharmaceutically acceptable salt thereof, a stereochemically isomeric form thereof or a solvate thereof.

30. The method as claimed in claim 27 wherein the compound is selected from the following compounds

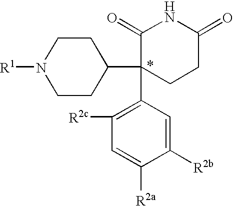

| | | | | stereo- |
|---|---|---|---|---|
| R¹ | R^{2a} | R^{2b} | R^{2c} | chemistry |
| 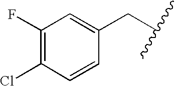 | F | H | F | *RS |
| 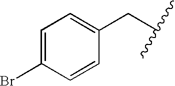 | F | H | F | *RS |
| 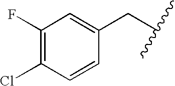 | F | H | F | *R; HCl |
| 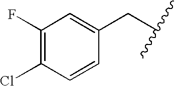 | F | H | F | *S; HCl |
| 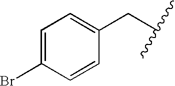 | F | H | F | *R; HCl |
| 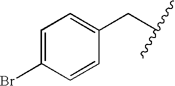 | F | H | F | *S; HCl |

a N-oxide thereof, a pharmaceutically acceptable salt thereof, a stereochemically isomeric form thereof or a solvate thereof.

31. The method according to claim 27 for treating a disease mediated through activation of the CXCR3 receptor.

32. The method according to claim 27 wherein the disease mediated through activation of the CXCR3 receptor is selected from the group consisting of rheumatoid arthritis, inflammatory bowel disease, allograft rejection, multiple sclerosis, COPD, glomerulonephritis, allergic contact dermatitis, lupus, psoriasis, atherosclerosis, Sjogren's syndrome, and autoimmune thyroid disorder.

33. The method according to claim 32 wherein the disease mediated through activation of the CXCR3 receptor is selected from the group consisting of rheumatoid arthritis, Crohn's disease, colitis, and allograft rejection.

34. A process of preparing a compound of claim 1 comprising
a) reacting an intermediate of formula (II) with an intermediate of formula (III) wherein W₁ represents a suitable leaving group, in the presence of a suitable solvent and a suitable base, 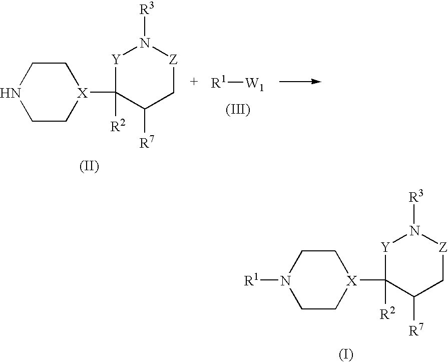
wherein X represents N or CH
Y and Z each independently represent C(═O) or CH₂ provided that at least one of Y and Z represents C(═O):
R¹ represents CH(R⁴)-aryl or CH(R⁴)-heteroaryl;
R² represents aryl² or heteroaryl:
R³ represents hydrogen; C₁₋₄alkylcarbonyl C₁₋₆alkyl optionally substituted with C₁₋₆alkyloxy C₁₋₆alkylthio, C₁₋₆alkyloxycarbonyl or aryl¹; and
R⁷ represents hydrogen or C₁₋₄alkyl;
b) reacting an intermediate of formula (IV) with a suitable acid, 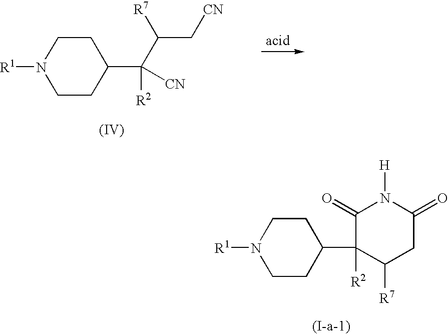
wherein R¹ represents CH(R⁴)-aryl or CH(R⁴)-heteroaryl;
R² represents aryl² or heteroaryl: and
R⁷ represents hydrogen or C₁₋₄alkyl;
c) reacting an intermediate of formula (XXXV) with HNO₃ 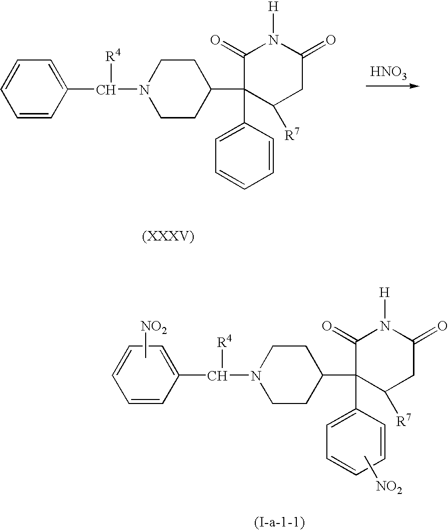
wherein R⁴ represents hydrogen or C₁₋₄alkyl and R⁷ represents hydrogen or C₁₋₄alkyl;
d) reacting an intermediate of formula (II) with an intermediate of formula (V) wherein R^{1a} represents aryl or heteroaryl, in the presence of a suitable reducing agent, a suitable acid and a suitable solvent, 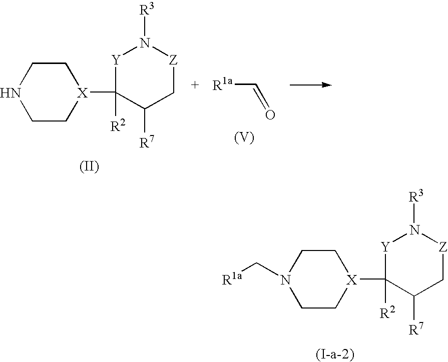
wherein X represents N or CH;
Y and Z each independently represent C(═O) or CH₂ provided that at least one of Y and Z represents C(═O);
R² represents aryl² or heteroaryl; and
R⁷ represents hydrogen or C₁₋₄alkyl;
e) reacting an intermediate of formula (VI-a) or (VI-b) wherein W₂ represents a suitable leaving group, with a suitable base of formula R⁵R⁶NH in the presence of a suitable solvent, 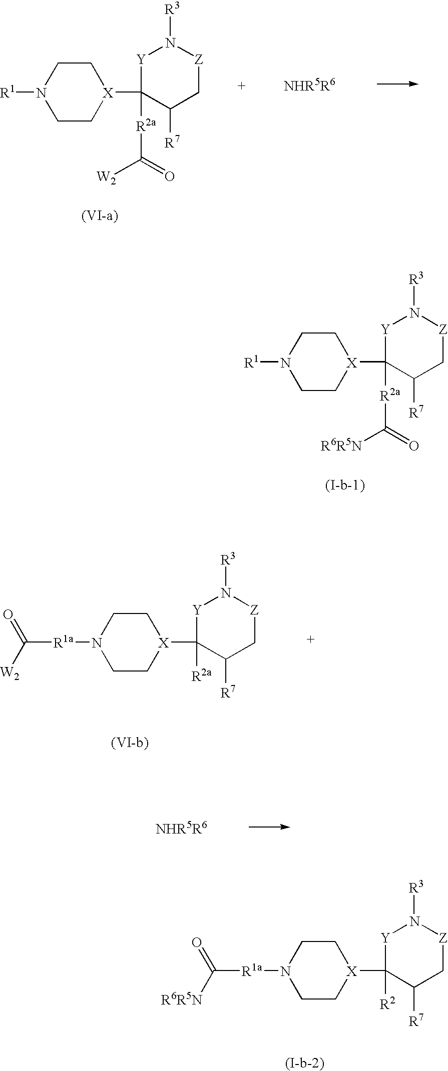
wherein X represents N or CH
Y and Z each independently represent C(═O) or CH₂ provided that at least one of Y and Z represents C(═O);
R³ represents hydrogen: C₁₋₄alkylcarbonyl; C₁₋₆alkyl optionally substituted with C₁₋₆alkyloxy C₁₋₆alkylthio C₁₋₆alkyloxycarbonyl or aryl¹;
R⁵ and R⁶ each independently represent hydrogen or C₁₋₆alkyl optionally substituted with hydroxyl; or
R⁵ and R⁶ together with the nitrogen to which they are attached form a monocyclic heterocycle selected from piperidinyl piperazinyl morpholinyl or thiomorpholinyl each of said rings optionally substituted with C₁₋₄alkyl; and
R⁷ represents hydrogen or C₁₋₄alkyl; and wherein —R^{2a}—C(═O)—NR⁵R⁶ represents a R² substituent wherein the ring moiety is substituted with R⁵R⁶N—C(═O)— and wherein —R^{1a}—C(═O)—NR⁵R⁶ represents a R¹ substituent wherein the ring moiety is substituted with R⁵R⁶N—C(═O)—;
f) deprotecting an intermediate of formula (VII) wherein P represents a suitable protecting group, with a suitable acid in the presence of a suitable solvent, 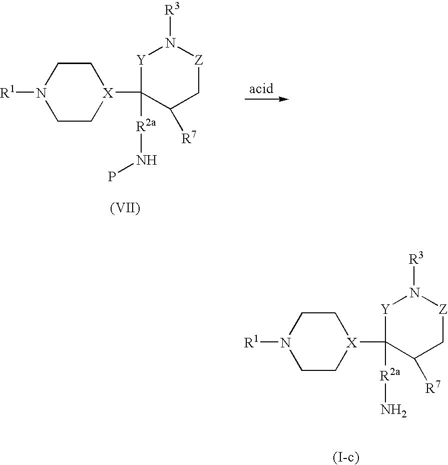
wherein X represents N or CH;
Y and Z each independently represent C(═O) or CH₂ provided that at least one of Y and Z represents C(═O):
R¹ represents CH(R⁴)-aryl or CH(R⁴)-heteroaryl;
R³ represents hydrogen; C₁₋₄alkylcarbonyl; C₁₋₆alkyl optionally substituted with C₁₋₆alkyloxy, C₁₋₆alkylthio, C₁₋₆alkyloxycarbonyl or aryl¹; and
R⁷ represents hydrogen or C₁₋₄ alkyl; and wherein —R^{2a}—NH₂ represents a R² substituent substituted with NH₂;
g) reacting an intermediate of formula (VIII) wherein W₃ represents a suitable leaving group, or an intermediate of formula (VI) with a suitable alcohol of formula C₁₋₆alkyl-OH, 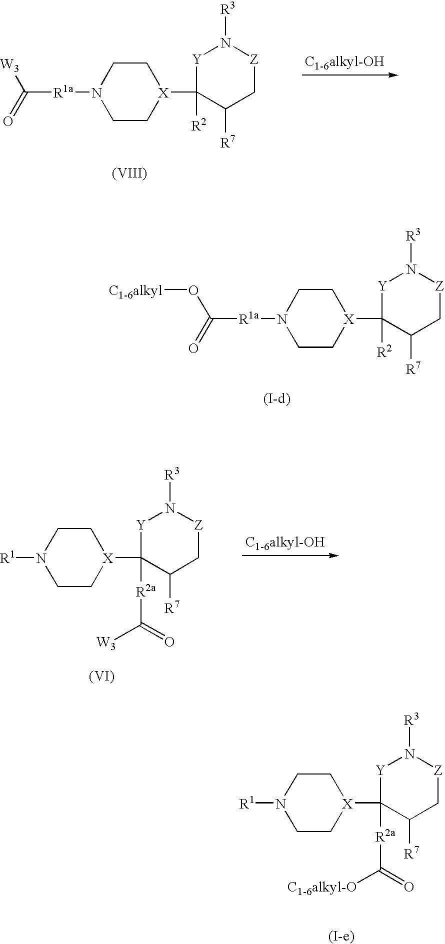
wherein X represents N or CH;
Y and Z each independently represent C(═O) or CH₂ provided that at least one of Y and Z represents C(═O);
R¹ represents CH(R⁴)-aryl or CH(R⁴)-heteroaryl;
R³ represents hydrogen; C₁₋₄alkylcarbonyl: C₁₋₆alkyl optionally substituted with C₁₋₆alkyloxy, C₁₋₆alkylthio, C₁₋₆alkyloxycarbonyl or aryl¹; and
R⁷ represents hydrogen or C₁₋₄alkyl; and wherein —R^{1a}—C(═O)—O—C₁₋₆alkyl respectively —R^{2a}—C(═O)—O—C₁₋₆alkyl represent a R¹ or R² substituent wherein the ring moiety is substituted with C₁₋₆alkyloxycarbonyl;
h) reacting a compound of formula (I-a) with W₄—R^{3a} wherein W₄ represents a suitable leaving group, in the presence of a suitable base and a suitable solvent, 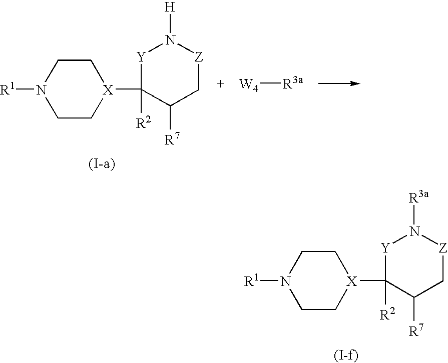
wherein X represents N or CH,
Y and Z each independently represent C(═O) or CH₂ provided that at least one of Y and Z represents C(═O);
R¹ represents CH(R⁴)-aryl or CH(R⁴)-heteroaryl:
R² represents aryl² or heteroaryl; and
R⁷ represents hydrogen or C₁₋₄alkyl; and wherein R^{3a} represents C₁₋₆alkyl optionally substituted with C₁₋₆alkyloxy, C₁₋₆alkylthio, C₁₋₆alkyloxycarbonyl, C₁₋₆alkylcarbonyloxy or aryl¹;
or, if desired, converting compounds of formula (I) into each other following art-known transformations, and further, if desired, converting the compounds of formula (I), into a therapeutically active non-toxic acid addition salt by treatment with an acid, or into a therapeutically active non-toxic base addition salt by treatment with a base, or conversely, converting the acid addition salt form into the free base by treatment with alkali, or converting the base addition salt into the free acid by treatment with acid; or, if desired, preparing stereochemically isomeric forms, quaternary amines, solvates or N-oxide forms thereof.
